SEQUENCE LISTING (35 sequences)

SEQ ID NO: 1 - DNA, 2061 bp, Pseudomonas stutzeri
  CDS (1)..(2061): ORF K
ctgggccggggcagtcgactcccgagcgcagcttacgggagtacgcatgatatgcgcggtcaaccgatgatgatggctacagctttgatctgtgcctttgtaccagggccacagttggcgtttgctgcgccaggctccgcggcttcgcctgactccacgacgctaccggaaatcaccgtcacagccgagaaaatcgagcggccgctggaaagggtgcccgccagcgtggcggtgatcgatggctgggacgccgagcagtcaggcatcactagcctcaaacaactggaaggacgcattcctggtctgtcattccagccgttcgggcaagcaggtatgaattcacccgtcatgcgggggctgacggccaacttcaacagcttctccagttcaacgttgttgctggtcgatggcgttcccacgctgacagcccagggattcgagagtggcatgctggatctcgatcgcatcgaggtcattcgcggcccgcaatctacgctgtatggccgtaatgccgaggccggtgtgattgccatccacagcctgccgatggacgcgaccccgagagccagcgtgtctgccgaagcgggcagccggaacaagcgtgtcatgcggtttgcgctcagccagcctttggtggaagagcggttgtacggcagcgtatcgggcaactggtcgagccaggacggcttcatcgacaacacccacacggggcacaaggcggacgatcgtgagcagaaraacctgaacctggggctgcgctgggccccgggggccgcaacggatgtggtcatgcgctatgcgcatcaggagtacgacgatggcgcctccctgtggggctcgcccggcgcgccaaggaagcgagtcgcgtccggaacgccgagctggaaccgttctgagggccagaccttgtccttcaatgtccagcatgaatttgcctccggcctgcggttgcattcggtaacggcctggaacgagttcaaggacaggattcagcaggacactgacttcatgccagccgatgttctgcacgtcgggcgcgaccatcacctgcgcacactctcccaggagttccgtgtggagggacagctcggggaggccagttggctggctggtgtctacgcggatcgcagcgacaacgatctgcacagtaccagcaagaccatgatggggctgtcggacattcgcgcggatcagcagagcgataccgctgcactgttcacccactggaacgtccccctgtcggccgactggtccatagacgccggagcgcgcgtcgagcgcaacgaggtgcagctacgtccgcaaggggctacgagccatgaaaaaggctggacacacgtttcacccaggctcgcgctgcaacaccagataaccgccaatcaccaatggtatgtgagtgccagtcgtggcgtgcgcactggcggcttcaatgtgctggcgccgacgctgggttatctgccttacgacacggagaagaactggtcgtatgaaaccggtctcaagggctggcttcttgacaagcgcattcgctattcgctggccgcctacctcatggacatcgatgacatgcaggtcatgcagatgcccaccgtcggcgtgatgtacatcaccagcgctgccacggcgacatccaaaggtctcgagctggatgtggactatctcctcggtggcggctggcagctcaagggcgggctggcctggaaccacacgcgcttcgatcactttcgcgatggcgaggcggactatgacggcaaccagaacccgttcgcgccggatctcaccggccacctcggcatccgctacgacgcgcccgaaggctggtatgcacaagccagcgtgaccggcagcagcaaggtctacctcgatgcggccaacgggtatgaacgcaacggctacggcctggtgaacctggtagctggttaccaacgcggcaactgggaaatcgcggcctacgccgacaacgcgaccgatcagcgctacgacgcggtgggctaccagaacggattcgtcaccgtctacagcccgccgcgagaagcgggcctgcgtctgacatggcgcctg

SEQ ID NO: 2 - protein, 687 aa, Pseudomonas stutzeri
LGRGSRLPSAAYGSTHDMRGQPMMMATALICAFVPGPQLAFAAPGSAASPDSTTLPEITVTAEKIERPLERVPASVAVIDGWDAEQSGITSLKQLEGRIPGLSFQPFGQAGMNSPVMRGLTANFNSFSSSTLLLVDGVPTLTAQGFESGMLDLDRIEVIRGPQSTLYGRNAEAGVIAIHSLPMDATPRASVSAEAGSRNKRVMRFALSQPLVEERLYGSVSGNWSSQDGFIDNTHTGHKADDREQKNLNLGLRWAPGAATDVVMRYAHQEYDDGASLWGSPGAPRKRVASGTPSWNRSEGQTLSFNVQHEFASGLRLHSVTAWNEFKDRIQQDTDFMPADVLHVGRDHHLRTLSQEFRVEGQLGEASWLAGVYADRSDNDLHSTSKTMMGLSDIRADQQSDTAALFTHWNVPLSADWSIDAGARVERNEVQLRPQGATSHEKGWTHVSPRLALQHQITANHQWYVSASRGVRTGGFNVLAPTLGYLPYDTEKNWSYETGLKGWLLDKRIRYSLAAYLMDIDDMQVMQMPTVGVMYITSAATATSKGLELDVDYLLGGGWQLKGGLAWNHTRFDHFRDGEADYDGNQNPFAPDLTGHLGIRYDAPEGWYAQASVTGSSKVYLDAANGYERNGYGLVNLVAGYQRGNWEIAAYADNATDQRYDAVGYQNGFVTVYSPPREAGLRLTWRL

SEQ ID NO: 3 - DNA, 1176 bp, Pseudomonas stutzeri
  CDS (1)..(1176): ORF N
atgaacgttgaaacccatcgtcctgcctcgctggctggactgtccgcgcttctgttgatagcgatgggcatgccgatgatgatcttctatgctatcggcatcctgggcccgcacctggttgccgacttggggatttcccgtcagcaactgggctggctgaccgccagcaccttcggactcgccgccctgctgtcaccctgggcaggcgcactggtccaacgcatgggcactcgtgcggggctgatatgcatgttcctgctggtggggttgtccttttcgctaatggcggtcctgcctggcttcggtggattggtcacggcattactgctttgcgggacggcccagtcattggcaaacccggcgaccaatcaggccatcgcgcatagcgtacccgttgcgcggaaagcgggtgtcgtcggtctgaagcagtcgggtgtgcaggcgtccgccttgttggcgggcgtggcgcttccaccgctggtgctgatgtggggatggcgtggcgcgttggcagcctgggtgcccgtggcattggtcatggccgcattggtgacctattgggtacctgcaaaatcggtgtcagcgccaagcctgccattgcgcgtgcgtggacccaatgtgtggctgtcgatattgatggccattcagctttgcgcaggtcttgcgctgtcctcgttcatgaccttcctcggcgtctatgccgcccagatcggcgtatcggtcagcaccatcggagccatggtcagttgctttggcgccatggggatcctgtcccgcgtgctgctgacaccgatcgctgacaagctgaaggacgaaaccattttgctcggcgtgctgttcatacttgcaggcctggcgctggcggtcatgcgcgaggccaacactcagcagcactggccactctggcttggcgtgacgggaatgggcctgaccgtggccgccagcaatgccatcgccatgagcatgctgttgcgcgatgggcgatttggcggcgcagccacttcggcggggatgctatcggttggattcttcggcggcttcgccgttggcccgcctgcattcgggtggttcttggcccactcagagggcttcgcggccgcctggcttagcctgatcggtatcttggtcgcaggcggtcttctgtgcctgctgttgctctacatgcgccatcgggaa

SEQ ID NO: 4 - protein, 392 aa, Pseudomonas stutzeri
MNVETHRPASLAGLSALLLIAMGMPMMIFYAIGILGPHLVADLGISRQQLGWLTASTFGLAALLSPWAGALVQRMGTRAGLICMFLLVGLSFSLMAVLPGFGGLVTALLLCGTAQSLANPATNQAIAHSVPVARKAGVVGLKQSGVQASALLAGVALPPLVLMWGWRGALAAWVPVALVMAALVTYWVPAKSVSAPSLPLRVRGPNVWLSILMAIQLCAGLALSSFMTFLGVYAAQIGVSVSTIGAMVSCFGAMGILSRVLLTPIADKLKDETILLGVLFILAGLALAVMREANTQQHWPLWLGVTGMGLTVAASNAIAMSMLLRDGRFGGAATSAGMLSVGFFGGFAVGPPAFGWFLAHSEGFAAAWLSLIGILVAGGLLCLLLLYMRHRE

SEQ ID NO: 5 - DNA, 1053 bp, Pseudomonas stutzeri
  CDS (1)..(1053): ORF P
gtgggtgacgtagcgcttatgaggtccaagatgaaatttcaacacgcccatccattgcaatcctgctgggatctcgcgctggcgtctgtgcaggctgatgcgttggcgactgcgctggatagcggccttttcgatgtgctgaccgaacaggcaggggccgggaaaattgcagagcagctcgaacttgatccgaacaggctcgagcccgttcttgagttgttatggagcatggatctgttggagcggtttcagctccatgacagcactgggtcgacgccacactatcgttccagcgacaaagcaaagcgttttttcgccaagcagtcgtccgattactgcggtgatgcttgggcctttcgccttcggtcgctacgcgactttggcacgcgattgccggaatatctgcaagtgcgtgccatcgaggaggcgccggtaccgaccgaccaatgctgggccacgcttgctcggacaaaaatcgtgcaggagcaatcggctgttacggtcgaagcggcactcgcgatcgttgctcgcctgccggagctaaagggcattcgacgtttcctggacctcggttgcggtcccggcatggtggccattgcgttggcacgcgctcttcccggatgccatggcactgcgttcgagttgccgccaaccgcagcagtggcacggcagaacgtcgagatggcgcaactcggcacgcgtctgtcggtgctgggaggcgacctgacccgtgatgaaattggcagcggttatgacctcatctggtgcgcttcggttttgcacttcgtgccggatcttgcgcagacgctacgtaagatccgagcagcgctggcgccgggcggggttttcgtgagcattcatgctgaaattccgctcacggcggcgcagacagccacggttctggcgtactacctgccgttgctgatgcgtggccaccatgtctggcaccagggagaactccccgaggcgttgttcgccgcaggattcgctaacgtcgccaccttcgagagcgacttgtttccattggcgccagtgcaagtgctggtgtgccga

SEQ ID NO: 6 - protein, 351 aa, Pseudomonas stutzeri
VGDVALMRSKMKFQHAHPLQSCWDLALASVQADALATALDSGLFDVLTEQAGAGKIAEQLELDPNRLEPVLELLWSMDLLERFQLHDSTGSTPHYRSSDKAKRFFAKQSSDYCGDAWAFRLRSLRDFGTRLPEYLQVRAIEEAPVPTDQCWATLARTKIVQEQSAVTVEAALAIVARLPELKGIRRFLDLGCGPGMVAIALARALPGCHGTAFELPPTAAVARQNVEMAQLGTRLSVLGGDLTRDEIGSGYDLIWCASVLHFVPDLAQTLRKIRAALAPGGVFVSIHAEIPLTAAQTATVLAYYLPLLMRGHHVWHQGELPEALFAAGFANVATFESDLFPLAPVQVLVCR

SEQ ID NO: 7 - DNA, 705 bp, Pseudomonas stutzeri
  CDS (1)..(705): ORF C
atgtcaccagcctggaattcattccagaagggctacaccaccatctcctcgttccagggtattcccggtgagcggcgctacgaggcggacgttacggtttcgcaactgcgcgtggtggcgtatgagccgctgatctgcaaatacgtgggcccggagcgggcggcggagacgctcggtggcaatcagctcagccgtctggcgtatcgcgccagcacgccagcgacaatggcccatgccaacgcgttgatcagccacctgttgccttgtagtcgtccgctctcgcggctggacctgcacatccacacccttagcctgctcaacgaacagctcagcctgctggcgccgcagcacggtgcagtttcatcgccgttttcgccagcggacatcgaccgtatcgagcaggcacggcagatgatggatgagcacctcgacaagcccctgacgctcgactatctagccaccatcgtcggcatcaacaagaacaagctcaaggacggcatgatctacctgtacaacgccacgccggcggagctgctgctggaattgcgcatgaccaaggctctcgcgctgctggaaaccgggctgcgggtttcccaggtcgcctggatggtaggctacaaataccccaacaatttcaccgtcgcctttactcgctaccacggcaaatcgccgaaagccttgttcggcaagcgactactggagccg

SEQ ID NO: 8 - protein, 235 aa, Pseudomonas stutzeri
MSPAWNSFQKGYTTISSFQGIPGERRYEADVTVSQLRVVAYEPLICKYVGPERAAETLGGNQLSRLAYRASTPATMAHANALISHLLPCSRPLSRLDLHIHTLSLLNEQLSLLAPQHGAVSSPFSPADIDRIEQARQMMDEHLDKPLTLDYLATIVGINKNKLKDGMIYLYNATPAELLLELRMTKALALLETGLRVSQVAWMVGYKYPNNFTVAFTRYHGKSPKALFGKRLLEP

SEQ ID NO: 9 - DNA, 408 bp, Pseudomonas stutzeri
  CDS (1)..(408): ORF G
atggcattgcttatcaagcgtcaggcgctggggcaggttctggctcaagcacgtcgcgatcacccacttgaaacctgtggaatcgtggcgtcttcactggaagcccagttagcgacaagagtaatcccaatgcgcaaccaggcggcatcacaaaccttctttcggctcgactcgcaggagcaattccaggtgttccgatctctggatgatcgcaacgagttccaacgggtcatctaccactctcataccgcgagtgaagcctatccgagcagggaggacatcgagtatgcgggctatccggaagcgcatcacctgattgtgtccacatgggagaacgcccgagagcccgcccgttgtttccggatacttcgtggaaaagtcatcgaagaaagtatctccattgtggaa

SEQ ID NO: 10 - protein, 136 aa, Pseudomonas stutzeri
MALLIKRQALGQVLAQARRDHPLETCGIVASSLEAQLATRVIPMRNQAASQTFFRLDSQEQFQVFRSLDDRNEFQRVIYHSHTASEAYPSREDIEYAGYPEAHHLIVSTWENAREPARCFRILRGKVIEESISIVE

SEQ ID NO: 11 - DNA, 270 bp, Pseudomonas stutzeri
  CDS (1)..(270): ORF H
atgtcgatttcagtgatcgttcccacattgctgcgcccgctgaccaatggggaaaagacagtttttacccaaggcaactcggtggcagaggccatcgagaaccttgaacaccagttccctggccttaaggcccggctggtcagtgcggaacatgtgcatcgtttcgtcaatatctacgtcaacgaagacgacatccgcttctcagatgggctcaacacgccactcaaggccggtgacagtttgaccgtgctgcctgccgtcgccggtggc

SEQ ID NO: 12 - protein, 90 aa, Pseudomonas stutzeri
MSISVIVPTLLRPLTNGEKTVFTQGNSVAEAIENLEHQFPGLKARLVSAEHVHRFVNIYVNEDDIRFSDGLNTPLKAGDSLTVLPAVAGG

SEQ ID NO: 13 - DNA, 25801 bp, Pseudomonas stutzeri
  misc_feature 8300,8319,8338,8708,11735,11768,18459,18686,18703,18711,18720,19226,20077,20506,20545: N = unknown nucleic acid residue
attcccggggatctgatcatctgcgagggacgcaatctgcacccggaggacatcgagcacactgtgatcgaggcgctgagcgacttgcgggcgcaaagctgcgcagtgttcagccatgacgacgaccagcagcgccagaccatcgtcgcagccatcgaactgaatcgtgaactcaagcgccgcctgcaggacaattgccgccagctcaaggccgtggtgcgtagcgcggtggtggacagtcatggcatcaccctcaaccgcatcgtcttcgtgcagccgaccagcattcacaagacgaccagcggcaaaatccagcgcgcgaagatgcgccagctctatctcgccgaggaactggacatgctgcaatgaaacccggtgccccttcagccctggtgcaagtggctggccgatgcggcggcaccagtgagcctggtctgtttccattgcgccggcggcaagccgcscaarakgtttttccccgtggaaaaaaggccgcccaaggcctttgcgagctgtatgccgtctgaactgcccggccgcagctcggcgcttgcgcgagccgttcgccgagtcgctggcgcaactggccgaggccttcgccgagcagtgtcgcgccctgccgaacaaaccgcttatcctcttcggtcatagcctgggcgcactgctcgcatatgaaaccgctcgtgtgttgctggccaaaggcgaaaggccacccgtgcagctgctggtgtcctcgcgccagagcccggactgkctgccggcctgcgcgggtctgccggcgctaaacgatcaggctctgcgcgattacctaggcaacctcgccggcaccccgccagaggtgctgcagagcaaggcgatgatggatctcgcggtgccggtgctgcaggccgacctgaagctgatcctcaattaccagcatcgacatctccagcccctgagtatcccggtgctggtcttcggcgcgatcagcgaccaacaggtgcgctatgagtcgctgctgagttgggagcggatcagcggcgaggggttcagcctgcggatgatcgagggagggcactttgcggtaatgcagcaaccgcagtgggtgcttgaccaggtgcaaaccgagttatagccctcccagccagctcccatacgcaggcacatctctgtttgcgcctcgctcttcgccctctcgcgttggacataakraactcgcccatgcgcgggggcaaaagatatcccaggtccttgacgtgtcgtgaatcgcgatccgatttgccgtcgaggctttttgccgtgttctcgaggcgagtagaagcgcaaccggggcttaaacttcattcgcctygccagacgggcccccgcttgcacttcatgaacttgcaaacctgacatgcaagggtgcacgcctgcgttttacccaccaatggcatgcccttatcaataccaatccgaaggtactggcgtgcgcgtgctcgtarctgataagaacgtccatcccaacaagccgtaaaaacggtcaaactgcgtttttgggcaatttaatcgccgcgatgctctagcaggggcagcacaattgggagcactcgaggacttcgatcccgaaccttcgcccggctggacaaagtgacacaagccgatgctagccccacggttgaaacccgtattcgctgctaccccgagcagcccggcaattatctgctgcgttggtcgttcaaatcctgtgaaactgatcctacgaaagccggtatcggcatatcgcaaggcctctggggcaaattttctatttcttgaccgcccccaaccggataccaaaagcttcgcgtttaccgcgggttttatcgagtctggacggcgtraaagggcaggcttgccctggacttggtgccggactactacattggtatgcagcgcctggcactttttgcatatttcgccatagcgacggggttcctatgctgacggcaactccacaggagtcgcccatgtcatttcatcacctgagagcgggttgcctgcccttgactgcttcgcagctttggcgaacgccacaccaaccgatccagtgctgggcgtgaagatcggctatctggcttatcgaccgaacgcggggccattgctctccaacgtcctcagcgaggcgaaaccatgatggcaaaacatttccctctactaagtttggtcctcgcgataccagttacgcacgctcagaccagtgatctgagctatgagagctttgtggatagcgatagacgcatcaaatgcctctacggctatgccgctgaaaagacgggtgatcacgagtcggcgatcaagattttcgaagattgtgtcgagcgctggaacgatgtgtattcgatgatttggcttgcccagatgtacgaatccggcgcaggtgtggatgtagacctggcgaaagccgcatcgctcatgagwacgcggcgcagagcggcccgacgatgttgcgtacgtcagtctggcacgctatcactatgggctcgcgctggcggaagggcgaggagtcagaaaagaccttcaagccgctcgtgcctggttgcaaagcgcctcaaaaggtggccagagcgaagcgaatgactacctcacgcaactggaaaagtcatcctctttgaacgctccggctcgtcaacaccctagcggagcsgaataaacgctttgccagctcctaccgagcgcgtcaggtgtatgacaacgtgctgcacaagcacagcaagtcccaggctcgcttcggtcgctcgggcctagacatgctggcgtacgaccctctggaagaagggcccttgtacctgttcggtgtgagcgcgagcgcccaagctaacggtgagcttcactacgacatctgagcttttttcggagttaggtgatgcgctgaacctggtcgaactccatggccgcatttacaaccaacctctgcgcataacgacgacatttatgtggtgatgatcagcgtgcgctcgagatcctcactttctcaggtggaacagggcgtgaagcgaaaatcgacatcaaatacacagtcaagcccatatcgcgaacgaccttcttccgaatgctccgacgcaagaccgaataggctctggtagatgtgcgccgatgctgacgctgagtagaaggatatcgagacagcagagatggactacccagaggtacctacggttttatccgagcatttacaaggccggagcccgatagccgctgattatgaagtttgacaaccgaccgcttttggcgaggtcgccgacgatacgccgcacaggccgccagcccagtacatgattttcgccgctgtcgttctcgacagcggcgggagggatgcatcattcagtacattaggctggagcgcccgtcatcgatcacgggcgcaccgaggtagcttacggctccagtagtcgcttgccgaacaaggctttcggcgatttgccgtggtagcgagtaaaggcgacggtgaaattgttgggggtatttgtagcctaccatccaggcgacttgggaaaacccgcaagcccggttttccagcaagcgcgagagccttggttcatgcgcaattccagcagcagctccgccgggcgtggcgttgtacaggtagatcatgccgtccttgagcttgttcttgttgatgccgacgatggtggctagatagtcgagcgtcaggggcttgtcgaggtgctcatccatcatctgccgtgcctgctcgatacggtcgatgtccgctggcgaaaacggcgatgaaactgcaccgtgctgcggcgccagcaggctgagctgttcgttgagcaggctaagggtgtggatgtgcaggtccagccgcgagagcggacgactacaaggcaacaggtggctgatcaacgcgttggcatgggccattgtcgctggcgtgctggcgcgatacgccagacggctgagctgattgccaccgagcgtctccgccgcccgctccgggcccacgtatttgcagatcagcggctcatacgccaccacgcgcagttgcgaaaccgtaacgtccgcctcgtagcgccgctcaccgggaataccctggaacgaggagatggtggtgtagcccttctggaattccaggctggtgacatcctgcccgtcgtagcaggagcggcctttcatcccaacggtgatgaccatgacccgaccttcatgcggtccgctcgtttcctcgatcagcggcacagtcggacgatagtgcagcctgccaagaccgaggcctggttcaagttcgacaaaatcctggaagcattggccaatctcgtcgggcagctgcagccgagtcttgccatcgctcaggacgctgcggatgcgtggttcttccattgtcagccggtagcagctcttgtgtgcgctcacctctcacctccagtccgttagccccccgaatgtcgcgcatatcaatctgtgtttctcgcatacaaaaaatgctaatggttctcattactataatgcatcattttaccgatggccagtcattggcgaccaaacacccaactaaaaatacggtaaaatcaatgtcttacaggccaggcactgcacgcccgagaggaactggacgactacccacgaagccaaaccctgtcgaaacactggcgttcccttctctccttgctcgaccgcacgcgctgcaatcgtgttggccgacgcagctgaccgagccgctgcgcgatggcaggccgtgtccggtctcacccgctcctgctggacggcggcagggatgggccgataccgcccgggtgaagatggctcaggtgcaggcggtcagggccgacgtgtcctgtgagattcttcctcgacagcccgtgttttcgccgataggacggacactgaacatggacggcatgcccggcttggtggcggtcgcactggcgaagccgaagctggaagtgtccggtgtagggttcgagtgcccggccgccgccgtcgtggcgcagcagcccatcgaaagcgtcggacttcccgaccgtctgagtgcgcgcagcggcgatctggtacaggaggtcgagcaacgggatgacgtctgttttccgttcgcttccgtcgctggcctgctcgcccgcaaagaggaaaacctctcatgaccaagtcgcgagtggtaggtctgcaactgttgttcggctggatgaatctggtgctggcggtacccagcatctacttgatgctcggcatgccacttgtaatgcgccagcatggctgragcggcgcagagatcgggttgttccagcttgccgcgctgccggcgatattcaaattcctgttggctgtgccggtgcagcgtgtgcgcctcgggcgcggacatttcgtgcactggttgctgttgctctgtgcgctactactggcgctgtactggctaatcggacggcataatctgatcggcgatcgcataatgctgttcgcgctgaccttcgccatcagcattgccgccacgtgggccgacattccgctaaatgcgctagcggtgcagtggttgccgcgtagtgaacagttgcgcgccggcagcatccgttccgcagcgctgttcgtaggcgccattgttggcggcggcgtcatgatcatggtgcaggcgcgcgtgggctggcaggcccccttctggctgctaggggtcggactgctgattggcgccctgcccttcctgctgttgcgtagacacgccgcactgcccgagcaggccgagccgcgcgagactacagatcctccaccgggcgtgatggcggactgggcaagcttcttccaccagccaggggcgcggcaatggacattgctgctgctgaccagtttccccttcctcggcgcgacgtggctgtacctcaaacctttattgttggacatgggcatgcagctagagcgcgtggccttcatcgttggcatcgtcggcggcaccgcaggcgcactgttcagcctgctcggcggacagctagtgcaaatgttgggcatagcacgggccattgcctggtacctgctggcggcgctgggcgcgctggcacttttgacgttcagcgtctgggcccaactgggggcggcatggctgattgccagcgccctctgcgtggcagccagcatgggcgccatctcggcgctgatgttcgggttgaccatgttcttcacccgaaatcggcgcaacgcgtcggactatgccctgcaaaccaccatgttcaccgttgcgcgactggcggtgccgatcgccgccggggtgttgctcgaccgggtgggctacaccggcatgctcctggcaatgaccctggcgcttctgctttccttcgcgctcgcctgtcgggtgcgggaaaaggtggaatcttcggcacagtcgatactcgagcacgagcgggtttgaaggctgaagtgaccggccatgccccttcggacaatggcctgaaatgcgcggtccttttgcatagtttttcatgctcacgtcatatgaaagaacagccaacggcaattgctatagtcatcaccacgaacgataatgattatcgttaccattgaaatcaaacaggataagcgatatgccactatcagcgctggtggcgccggctggggaactgagccgcgccgagatcaaccgttacagccgccacctactgatacccgatgtgggcatgatcgggcagcgtcggttgaagaacgccaaggtgttggtcatcggcgccggcggtcttggctctccgactctgctctatctagctgcagcaggtgtgggcaccatcgggataatcgactttgatcgggttgacgactccaaccttcagcgccaggtcatccacggggtggataccgtgggcgagctcaaggtggacagtgcaaagaaagccattgcgcgactaaatccctttgtccaggtcgaaacctataccgatcgcctggaacgggacatggcgatcgagctgttttcgcgctacgacctgatcatggacggtaccgacaacttcgcaacccgttacttggtcaacgacgcctgcgtgctggccaacaaaccctatgtgtggggctcgatattccgtttcgaagggcaggcgtccgtgttctgggaaaacgccccgaacgacctgggcctgaactaccgcgacctgtatccggagcctccgccgcccgagatggccccctcgtgctccgagggcggtgtgttcggcattctttgcgcatccatcgcatcgatcatggccaccgaggcggtcaagctgatcacgggcatcggcgagccactactgggtcggctggtggtgtacgacgctttggatatgcgctatcgggagcttcctgtgcgccgcctgccaaatcgacaaccgatcaccgacctggccgaggactatcaggtgttctgcggtctggggttgcccaaaggtgacacggcggacgccgtgccagggatcagcgtaacggaactcaagaagcggatggatcaggacgaggtgcctgtgctgatagacgtgcgcgagcccaccgagtgggacatcgtccgtattccgggcgcaatcttggtgaccaaatcgcccaccgcagcgcagacactgcgcgagcgatacggggcagatgccaacctggtgatcgtctgcaagtccgggcggcgctctgccgacgtgaccgccgagttgytaaacctgggcatgcgcaatgttcgcaacctcgaaggtggcgttttggcctgggtgaaggacgtggactcttctctgcctagctactgataggaggcctagaagatggcattgcttatcaagcgtcaggcgctggggcaggttctggctcaagcacgtcgcgatcacccacttgaaacctgtggaatcgtggcgtcttcactggaagcccagttagcgacaagagtaatcccaatgcgcaaccaggcggcatcacaaaccttctttcggctcgactcgcaggagcaattccaggtgttccgatctctggatgatcgcaacgagttccaacgggtcatctaccactctcataccgcgagtgaagcctatccgagcagggaggacatcgagtatgcgggctatccggaagcgcatcacctgattgtgtccacatgggagaacgcccgagagcccgcccgttgtttccggatacttcgtggaaaagtcatcgaagaaagtatctccattgtggaatagcgactttccaatatttcaatcagcaatgcctcagccacagctagaggcaaaaggagttctacatgtcgatttcagtgatcgttcccacattgctgcgcccgctgaccaatggggaaaagmcagtttttacccaaggcaactcggtggcagaggccatcgagaaccttgaacaccagttccctggccttaaggcccggntggtcagtgcggaacatgngcatcgtttcgtcaatatntacgtcaacgaagacgacatccgctttctcagatgggctcaacacgccactcaaggccggtgacagtttgaccgtgctgcctgccgtcgccggtggctgactcgcacctccggacaccgctgaaagaatgacccctggcgattcaaatccaggggcaagtgcaacgcttttgtctctcggttttgaatatgccgacactcttaaatgaattttccctgctgcattcatccacttcgtttccgccgaattggaatgaactgcaacttagcctgacggaacaggccagattattgggcatttgcccgctcgcaatctcgccgcctgtgratatggaaggagccgcattccagctgcagcatccggctatttntcctattcaggcccacttcgcctcaccagccggctggctgccaaatcgacacctctcggagctgctgctgcaggcgggcagcggtcttatgtcggtgcacggccgtgctagcggtagggcccaaccgctgggcgtggattatctttcgacacttaccgccgtcatgacgctgcacggaacgctggccgcagccgtggggcagctgcgtggcggtgcatttgatcaggttcagctttctccactgggatgcgggctgctcagtatcgggcagtatctggcaggcgccacggcaccagaagatcgtgaggcgttcctgccgggcggctccgatccgcatttgaggccgccatttcgttccgctgacggcatcacattcgagctggaaacgctcgacagcacaccgtggcgaagcttttggaccgccgtcggcattgaatcggaattggccggtacggcctggaaaggttttctgcttcgctacgcgagggccgtgtcgcctctacctgccgcctgtctcacggcgctcgcccgcctgcgttacgcaaagatccaacaattggcagcgcaagcgggtgttgcggtcgtgcccgtccgcaccgatgcgcaacgccgcgaggaccccgattaccggcagtcactggctacgccatggcagttcgagtctttcccgccgtcccccgaaaggcatcgagacaccgcatttccgtcactgctgccgctacaggggatgcgcgtcatcgaatcctgtcgacgcattcagggaccgctggccgggcatctgctggcatcgctgggcgccgaartcattcggctggarccgccgggtggcgatccgttgcgarccatgccgccctgcgccgaaggctgttcggtgcgctttgacgcgctgaaccacctcaaatccgttcacgaagtcgatatcaaatccgcccatgggcggcagttggtctacgagctcgcccgcgatgcggatgtctttctgcacaactgggcgcccggcaaggcccatgaaatgcaactggatgctgaacatctgcgcagggttcaaccacatctcgtttacgcctatgcgggaggctggggccgggctcccgtcaatgccccgggtaccgacttcaccgtccaggcctggtcgggtgtgtccgccgccattgcacgtcaatccggcatccgcggcggctcgctgttcaccgtgttggatgtgctgggcggcgcgatcgcggcactgggtgtgacggccgcgttgctcaatcgagcagtcacgggcacgggtacttatgtcgagagctcattgctgggcgccgccgatctgctgatgcacagcagcggcaaggcgtcgaggggcatcttgtccggcgtgtatcccacgctatcgggactgatcgccatcgactgccaacacccagatcagttccagtcgctggccatgttgctggacattcctgccactgcgratacctgccagcagacgctgcggagcgcttacgcaagcgacccgcttcggaatgggaaacggtgctgaacgaacggggcatcggcgcctgtgtagtcatcgaagacctcaagcagctcgccgccgacacccgcatctctgaatgcctcactcgcaagtcttacttctctgtcaacgccccctggaggttcctatgaacaacgctggcatcatcgacctggttcctgctgaggaacgccaacgttgggtgcaggacggtacctacccgaaccagcccgtattcacgctgtttgccgccaaagccgaagcgcatcccgacaagaaggccgtgctgtcgccgcaaggtgacgtgacctacggcgagctcctcgatgcagccctgcggatggctcacagcctgcgtgattcggggatcgtggccggcgacgtggtggcttaccagctcaccaaccactggttgtgctgcgcaatcgacctggcagtggcagcgctcggtgccatcgtcgcccccttccctccgggacgcggcaagctggatatccaatcgctggttcgccgctgcgacgcgcgagcggtgatcgtcccgcaagcgtacgaaggcatcgatctgtgcgaggttatcgagtcactgcgccccaccctgctatccatgcgccgcctgattgttcagggcaagcctcgcgaaggatggattacgctcgatgagctgatgagcaccgagccgctggatctcgccagcctacccagggtgtgcccgaactcgccggtgcgtctgctggtgtcttcaggcaccgagtcgragcccaagctggtggcgtactcgcacaatgcgttggttggtggtcgcgggcgcttcctgcagcgcatcgcgtccgatggcgaagattttcgcggcatgtacctcgttccgctgggttcgtccttcggctccactgccaccttcggtgtgttgtgctggctgggtggttcgctggtcgtattgcccaagttcgacgtggatgaagccatcaaggcgattgcggcatttcggccgggcttcattctcggcgtacccaccatgctgcaacgcatcgccgctcaaccggcgttggagagcatcgacaaatccagcctgcgtggtttgatcgtcggcggctcggtcatcgacgaggccaccgtgcgcaaatgccgtgatgcgtttggctgcggcttcatcagcctctacggttccgccgacggcgtgaactgccataacaccctggacgaccccatcgaagttgtgctgaccagcgtcggcaagcccaatccggcggtctgcgcgattcgtctggtggacgacgaaggccgggaggtccggcaaggcgaggttggcgaaatcaccgcccgcgggccattgactccaatgcagtacgtcaacgcgccggagctggacgagcgttaccgcgacccgcaaggctgggtgaagaccggagatctgggctacatcaacgacaagggttatctggtcctagccggtcgcaagaaggacgtcatcatccgtgggggcgccaatatcagcccgacccagattgaaggcctggtcatggcgcatcccnatgtcgtgaccgttgcgtgcattcctgttcccnatgatgatctcgggcagcgggtgtgcctttgcgtcacctttgcgcgagggtgcagcgaagttttccctgaaagcgatcaccgacttcctgcgcgaactgggactggaggtgaacaagctccccgagtacctacgcttctaccgcgctctgcctctgacaccggcgggaaagatcgataaaaaagcgctaaccgaggaagcccgcgagctgggcacctcgggcatttgtcccgctgggccggggcagtcgactcccgagcgcagcttacgggagtacgcatgatatgcgcggtcaaccgatgatgatggctacagctttgatctgtgcctttgtaccagggccacagttggcgtttgctgcgccaggctccgcggcttcgcctgactccacgacgctaccggaaatcaccgtcacagccgagaaaatcgagcggccgctggaaagggtgcccgccagcgtggcggtgatcgatggctgggacgccgagcagtcaggcatcactagcctcaaacaactggaaggacgcattcctggtctgtcattccagccgttcgggcaagcaggtatgaattcacccgtcatgcgggggctgacggccaacttcaacagcttctccagttcaacgttgttgctggtcgatggcgttcccacgctgacagcccaggggattcgagagtggcatgctggatctcgatcgcatcgaggtcattcgcggcccgcaatctacgctgtatggccgtaatgccgaggccggtgtgattgccatccacagcctgccgatggacgcgaccccgagagccagcgtgtctgccgaagcgggcagccggaacaagcgtgtcatgcggtttgcgctcagccagcctttggtggaagarcggttgtacggcagcgtatcgggcaactggtcgagccaggacggcttcatcgacaacacccacacggggcacaaggcggacgatcgtgarcagaagaacctgaacctggggctgcgctgggccccgggggccgcaacggatgtggtcatgcgctatgcgcatcaggagtacaacaatggcgcctccctgtggggctcgcccggcgcgccaaggaaacaagtcccgtccggaacgcccaactggaaccgttctgagggccagaccttgtccttcaatgtycagcatgaatttgcctccggctgccgttgcattcggtaacggcctggaacgattcaaggacaggattcagcaggacactgacttcatgccagccgatgttctgcacgtcgggcgcgaccatcacctgcgcacactctcccaggagttccgtgtggagggacagctcggggaggccagttggctggctggtgtctacgcggatcgcagcgacaacgatctgcacagtaccagcaagaccatgatggggctgtcggacattcgcgcggatcagcagagcgatacgctgcactgttcacccactggaacgtccccctgtcggccgactggtccatagacgccggagcgcgcgtcgagcgcaacgaggtgcagctacgtccgcaaggggctacgagccatgaaaaaggcwggacacacgtttcacccaggctcgcgctgcaacaccagataaccgccaatcaccaatggtatgtgagtgccagtcgtggcgtgcgcactggcggcttcaatgtgctggcgccgacgctgggttatctgccttacgacacggagaaraactggtcgtatgaaaccggtctcaagggctggcttcttgacaagcgcattcgctattcgctggccgcctacctcatggacatcgatgacatgcaggtcatgcagatgcccaccgtcggcgtgatgtacatcaccagcgctgccacggcgacatccaaaggtctcgagctggatgtggactatctcctcggtggcggctggcagctcaagggcgggctggcctggaaaccacacgccgcttcgatcactttcgcgatggcgaggcggactatgacggcaaccagaacccgtttcgcgccggatctcaccggccacctcggcatccgctacgacgcgcccgaaggctggtatgcacaagccagcgtgaccggcagcagcaaggtytacytcgatgcggccaacgggtatgaacgcaacggctacggcytggtgaacytggtagttggttaccaacgcggcaactgggaaatcgcggcctacgccgacaacgcgaccgatcagcgttacgacgcggtgggctaccagaacggattcgtcaccgtctacagcccgccgcgagaagcgggcctgcgtctgacatggcgcctgtgaacaaagtcgcyggaatccgcgggatcgggcaatggcagcaagccaaagaggatgcgggcatatgcagcaagagagacggaatatcggaatactcttggtcagccaggatgaaaaactggcgttggacctggacatggtagtggaaagcgtaaacggtttgctcagtcgcaataccgatacgcctttcgatttgcacccgaacgacgaatgctttccctaccgccagatcttcgctcagrcctgtcgatacttgcgctcacaacccagggacaagctgcccgccctgttctctcggtgcttccactcaatggcaaccgcccgccaggcgctgtcggcgggtgattggacgttgtccccgcttgaatgcgtgttgctcgatacgcgccatgaggaaccggtcgatgacgaccccttccttgcgcttacgctgcaacaaccgccgacccggccctcgccctgctctgcaatgctgctgtgcgaggaaagggtgctgggtaaatggatgtgccggctggggggcaaccgcctggtgcgggtgccgcattcgaatccctggcaacgccgcgccgatatyctccsggctgattctggatcatctggagcatgcccatttcaacccgcatgcttgctcgcscgcgccagaaagccgatggccaggtgacgctggcacaaaagattcatcacctcatgactgaacgctgggggtgatcaatgggacttccacttctacacgggctccatggtcgccggtttcatcgactcgatgaaatctctgctgcagggaacggacagccactgcctgaccggtaacaacgagcactcattggccgtaagcgcgctggctggctggcagttgtatgrycgtgcctacgttcatcgccattgactccgggaatgatcgacgaagcgcgcgggaactctggcgaatctcaagcgtgccgcaagcccctggcatcattgtctgcgccgactcmcccggaaacgatctggtatccattccagggtacgctcgacgccgacagcgacggacatgcggtcattgcagcacgcggcttgtggcatgggttcatgcgtacccccgatgacatgccagcttgccttgcaaatgccttccaggcccttgatgagcgccccgctccaaccttcgtccttgcaacgcaacacgtgctggagtcgcaaagcgaccatttcggaaccagtggtgcagcgcccctcctccaaggctgcaacgctctcctgcgcgcaacgcgaacgactggatcaggccgtggccgccatcaaccacgacaacgcccggatgctgtggcattgcggtcgcctcacatcggacgagcggaatcagattcttcgcctggccgaaaaagccggcatcgccctggtggacagcatcattcatccgggaagcgtgcccgggttcagcgacggcaagtccgtagcgaactatctgggaacgctttccatgtatggattcaaccgcgcggtctatgaattcctggaagcgcaaagcaccaacgaagaaggcgctccctggctctttttcctcaagggcaaggtagagcaatcgtctaccccgtattcggaaggcaagctcaagcgcaacttccgcattggccaagtcaactgcaatgaggcgcacctgtcgccattcaccctgctgggactggacgttcggttggcggacttcctgaattacctcgaaccacgcctgcaggttgacgacgcggtgctacggcagcgccgcgccagaatcgaacaattgcgcaagctgccggccgctcagccaagcgacctgatcgaaacaatgccgatgaccccgaattatttcttccaccagttgggacgactggtggtcgatctgatcgagtcgcgaagctaccgctataccggcgtctacgacgttggtcgttgcggtttgtcggcaatgcgcaacgtggcacgcaccgatcccggtttctccggctggtacggtcgcgccctgatgggtgacggtctgatgtcgctgccctatatcgccttgaagaacgaacgcaatgtgctggcattcatcggtgatggtgctcgcgcaatagtgccgaatgtggagcagcgcctggttggttctttcgatccggggcatcgcagggcgtggtggcaacgtcaccgttttctatctgagcaacggcgtgctttccatgattcagacctatctggacaaacgctacacgctcaatggctgcagccaggtcaacgtgccattgacggaatggaaggaggctcccgtcgagcatgctgaagacgatgtgacggttcatcgtcgcgttatccggcaattctgcccggcgctgttgggcgaggccttgatggctccgcgccgcgtcaatttctttgatgtctggcttggacacaactcggagggcgatggcttgagcctgatctcggaagggtcctggagccgcatgcgtactcatggagagtgaacacattatgaagttcggttttattgcccaccccacatcccgggacttgctgcatcaggtcaagctgatcgatatggcgggtcgtatgctcgaggaacaggccaacggctacgacggcgagcgctggcgccgtcgcaatctggttccgttcattgaattcactcgcatcgtgagcgccagcggttcgcagtgtgaaggcatcttgcagttcatgccgttgacgcagagcaaatgttgagtcagccgaggcgcattgcggagcgtgtcgtcgaaggcgtcaatgcgttaaaggacgagggggcagaactcgtagggctgggaggcttcacgtccattgttggcaatcgcggcctgcagacactggatcgcacgcaaattccggtgacgacgggtaactcgctgactgcgtatgccgcctatatgaatctgttgggtgtcctatccgcgctggaaattccaccagaaaaagcggaggtcgccgtgctcgggtatcccgggtcgattgcactggccatcgtttgcctgctggcgccattggggtgtcggctgcgactggtgcatcgcggagggaaacagcaggtcgacacgttgcttgagtacctgccctcacaatttcacggacaagtgacgctgcacgccggtctggaagattgctatgccaggtccgattgttcgttgccgcgacctccaccggtggggtcattgatcctcggcggctggcttgcggtagtgtcgtggtggatgcagcgttgccgaaggacatgcaacctggctgggaaaaacgcgacgacattctggtcattgatggcggcctggtctctgcgactgacgcagtggacttcggggccatggcgctgggcctgggtcctaagcgcaatatcaatggttgcctggccgaaaccatgatcctggcgctgcagggtcgcgcagaggcattctccatcgggcgcgaactgcctgctgagaaggttctcgagatcggtcgcatcgccgagggacatggctttctgccttaccccatggcgagcggcggcgaatcagtggatggcgctcggttcgatgaactgcgacgatttcacggtgccaggccgccagctgtcgtacacgacctgaataccggctcccaggagcttcgctcagaagtgctgcgctgcttcggtacgcacatcaaccccattctgcgtgaattctatgagttcaatcatgtcgagcgtgtcttcagcaatggccaaggctgctggctgacggatctggacggtcgccgttacctggatttcgtagcgggatacggctgcctcaacacgggccacaaccatccggagatagcagccaggctgcaggaatacctcacccagcagcatcccaccttcgtgcagtacctgtctgcgccgttgcatgccagcctgctggccaaacgtcttgccgagctggctccagccggactggagcgcgtgttccttagcaactctgggaccgaagccgtggaggcggctttgaagctggctttggccgccagcgacaaatccacgctgctctactgcaccaatggctatcacggcaaaacgcttggcgccctgtccgtaacggggcgtgagaagcaccgcaaggcgttcgaaccgttgctgccgcgctgcgaggagattccgttcgccgatgtttcggcgttgcgaaaccggttgctcaagggggatgtcscamccttcatcatggaaccgatccagggtgaaggtggtgtcaccatggccccggacggctatctcagagttgtgagggacctgtgctccgagcatgaatgcctctggattctcgatgaaatccagaccgggctcggacgtaccggcaagatgttcgcctgcgaatgggaagacgtctcgcccgacatcatcgtgctatccaaatccctgtcgggtggtctggtgcctatcggngcaacgctgtcctcgaaagaagtctggcaacgcgcctacggcaatatcgaccgattcgcattgcacacctcgacgtttggcggcgggaattttgctgccgccgccgccatggccgcgctggacgtgatcgagcacgaagacctgcccggcaatgccgctctggttggtgcacacctgcgacaagggctggaggcgctggcccgcaagcactatttcatcaaggaggttcgcggcgangcctgatatcgccatcnaatttaanaacgatgtntcaaatggcattgaagccttcgtgcgggatatgaccagccggatgcccgccaatgccgcagccacctaccgaatgatgcctgccaaggcccgcgaacacctcgaagcagccatgcgcgaattggagtccacgctggccgacatgttcgtcctgcgcatcatgaccaagttgtcccaggagcacggcatcctgaccttcgtgacggccaacaacaaccgtgtcatgcggattcagccgccgctggttctatcactggcagaagccgatcggttcataaaggcattgggcgaggtctgcgaggatctctcgacctttgagtcatgagttcagtcatcgggatgcgctgttgattcgatctccccttcccagcttataagggtgacgcaatgaacgttgaaacccatcgtcctgcctcgctggctggactgtccgcgcttctgttgatagcgatgggcatgccgatgatgatcttctatgctatcggcatcctgggcccgcacctggtngccgacttggggatttcccgtcagcaactgggctggctgaccgccagcaccttcggactcgccgccctgctgtcaccctgggcaggcgcactggtccaacgcatgggcactcgtgcggggctgatatgcatgttcctgctggtggggttgtccttttcgctaatggcggtcctgcctggcttcggtggattggtcacggcattactgctttgcgggacggcccagtcattggcaaacccggcgaccaatcaggccatcgcgcatagcgtacccgttgcgcggaaagcgggtgtcgtcggtctgaagcagtcgggtgtgcaggcgtccgccttgttggcgggcgtggcgcttccaccgctggtgctgatgtggggatggcgtggcgcgttggcagcctgggtgcccgtggcattggtcatggccgcattggtgacctattgggtacctgcaaaatcggtgtcagcgccaagcctgccattgcgcgtgcgtggacccaatgtgtggctgtcgatattgatggccattcagctttgcgcaggtcttgcgctgtcctcgttcatgaccttcctcggcgtctatgccgcccagatcggcgtatcggtcagcaccatcggagccatggtcagttgctttggcgccatggggatcctgtcccgcgtgctgctgacaccgatcgctgacaagctgaaggacgaaaccattttgctcggcgtgctgttcatacttgcaggcctggcgctggcggtcatgcgcgaggccaacactcagcagcactggccactctggcttggcgtgacgggaatgggcctgaccgtggccgccagcaatgccatcgccatgagcatgctgtngcgcgatgggcgatttggcggcgcagccacttcggcggggatgctatcggttggattcttcggcggcttcgccgttggcccgcctgcattcgggtggttcttggcccactcagagggcttcgcggccgcctggcttagcctgatcggtatcttggtcgcaggcggtcttctgtgcctgctgttgctctacatgcgccatcgggaatgagtccatgcaaggaaataacctccatgaagttgccatccgcgccatcttcaagcgaatggatgaaattgccgcgcaatgcggcgatcgtttcccgttgttccgccgcagcgcagacggactgtggacgttatccacacgaggctcttggctgggtgggttctgggcggggctttggtggcggcgcgccgcctacacaggcagtgcagacgaccatgctgtgncagaagcttggtcggcgcgacttgccccattgttacgtnagccatcgatcaatcgcagtttcgttttctggtatggcaccgccatcggccatgagaagcgcggtaaagaccaagcgtcccagcaacttgccaaccaagcggctctcgccatctctgaaagtttcaaccccggattgggtggctggatgtccggttccggcatgggcgccggagaactcggaacgcgcgcgctcaacgtcgacgcgctggcgccgacactggcactgctgcacggtcatggaggccagaaagaacgcaaacgggcgcgtcagcatttacagacctgccttcactacctcgcaaccgagcatggcgcctggcgaaccaatgctgttctagatgccggtgacggcgcgaaacaggaagcggccggggcttggcctcgcggacaggcctgggccatgctgggcatggctgaagcggttcgattgtttggagaagcctatcggcatccggctttgcaggcttgcacgtattggactgaacgctggggaggcgccagtcagcctgcagaaacatcagggttgcaacgcccgccgcaggacctttccgcgcacgtgatttcggcaactgccatgctcaatctgtcgcggctcatcccaggccagcattggttgtaccgccaggcacatatgcaaatttctgcggtgcttgatgtcactgacgttgtcgagacagggcaattcgttgggcatctttatcacgtcggcccagatgatgaacagctggttgagtcggcctgcgcgacattcttcttgcttgaaaacctgattaacctgtaacgcgctgtgctcgttgcggttcgcgccgacgtcccgaggggcctgcccaacgcacaccgcatctggcgctacaggccgtcttcgcctctcaccgcggtcggccttcccccaaacgcagcgccgtccctcgccccagggcatctggtgcgtattagcgctggcaccatttcgcgccactgaacgatacgctcagggcctgagaggcttcaaagcagcgaaattcctctttcgagcaggcggcgcgcgatcatgatccgatgaatttccgaggtaccatcgtaaagccggaagaccctcgcatcgcggtagtagcgctcgatcggcaactcagtgcaatagcccattccgccaaagatctgcactgcgctgtccgcgacgcggcccagcatttcggacgcgaacaacttcaccatcgaaaccttctcacgcacatcccgcccttgatcgatttcccaggccgcgttgagaagcatcatcctggcgccgtagatttcacactgcatgtccgccagcatcttctgcaccatctggaaactgccgatcggggcaccgaactgcgagcgctgcttcgcgaaatcgaccgacatttccagtagtttcgaagccatgccgacagccctggcaccgacataggccaaccgtgcaacgttgagatggccaagtaccagcgacatgccccgccccggctcgccgagcaattgttgcggcgcgatccggcagtcggtgaagaacagcccgtgctgatgggtaccgcgatgccccatcatttcctgtatcggcccgagttcaaggcccggcgtatcgcggtcgaccaaaaagcaggagataccttcctcgctgcgtgcagtgacgatataagcgctcgcgatatcggcatcgctgatgaagtgtttgctgccgttcaatatccactcgcaaccgtcgcgcttgaccgcggttctgatgccgtttgcgtccgaaccggcctcgggctcgctcatggcaaccgacacatgtatatcgccacgcatggcggcatgcaggtacttctcgcgctgctcgccggtacaatgaacaaggatgctgggaacgtggccgaatgcccgccttgccaacacatccttggttcggccgatctgctcattgaccagacagaattcaacggcactcagcccgccgccgcccacctcctgcggcatatgcatggcccacagccccagctcccgggccttttgcttgaggccttcagccacgctgggatcgatcgcattcgttctttcgatctcatcctcgagcggattgagttcgttttcgacgaaccgcttcaccgtcgaaaccagcatttgcacttcattgggaagggaaaaatcgatcatgtcgttggcttccggcgttccggccaagcgtgaaagccggcctgatggatcaagttgccagcgaggcggtaactggcccaagggaacaccacgtgctcggcgacgcttgtgcgggtggtgtagcagatgccgtaggtgacacgacacaaagacctgcctacggtttcgtcgcgcacacgttgctcgctgcggatgatggcaatgaacgcggagtcggcctgcttttccagatcggctccatcggctagtccgagggccaaagacatcatcggcacaccagcacttgcactggcgccaatggaaacaagtcgctctcgaaggtggcgacgttagcgaatcctgcggcgaacaacgcctcggggagttctccctggtgccagacatggtggccacgcatcagcaacggcaggtagtacgccagaaccgtggctgtctgcgccgccgtgagcggaatttcagcatgaatgctcacgaaaaccccgcccggcgccagcgctgctcggatcttacgtagcgtctgcgcaagatccggcacgaagtgcaaaaccgaagcgcaccagatgaggtcataaccgctgccaatttcatcacgggtcaggtcgcctcccagcaccgacagacgcgtgccgagttgcgccatctcgacgttctgccgtgccactgctgcggttggcggcaactcgaacgcagtgccatggcatccgggaagagcgcgtgccaacgcaatggccaccatgccgggaccgcaaccgaggtccaggaaacgtcgaatgccctttagctccggcaggcgagcaacgatcgcgagtgccgcttcgaccgtaacagccgattgctcctgcacgatttttgtccgagcaagcgtggcccagcattggtcggtcggtaccggcgcctcctcgatggcacgcacttgcagatattccggcaatcgcgtgccaaagtcgcgtagcgaccgaaggcgaaaggcccaagcatcaccgcagtaatcggacgactgcttggcgaaaaaacgctttgctttgtcgctggaacgatagtgtggcgtcgacccagtgctgtcatggagctgaaaccgctccaacagatccatgctccataacaactcaagaacgggctcgagcctgttcggatcaagttcgagctgctctgcgattttcccggcccctgcctgttcggtcagcacatcgaaaaggccgctatccagcgcagtcgccaacgcatcagcctgcacagacgccagcgcgagatcccagcaggattgcaatggatgggcgtgttgaaatttcatcttggacctcataagcgctacgtcacccacaggcctacctcactcagcaggctgttgatcgtggtcgcgccatcaatgccgatggtgtcgtggtttcgatacccagcgttatggaaaaaaaaaggacgactcgtgtgtccgacggtgcaatccgccatcagttgatcgcgtcgtattcggcgccgccgttgtcgaaggtctcgaactgcccatgtgtcaatgcgccgaagctcgccagcagcgcgtgaagtcaaagacgtctggcatgaagctttatgcgaagtgtcaccattgcccgctcccaaaaggtgttttcgccaaatcgctctcgtcgcgcggaggaatcctgggcacttgaaccatgtgctccatgggcttgacggccaatccatggcagataacgcagtcaacaatagcaatatctcgcattcccattcgtattaggatcgctgtatttcgtatgcgatccgtgcaactgggcgcgcggcgcgagaaaagctcgtacctttaccgggcttcgatcattcgtcatcagcaagcaacggcaggacgaacgcggtggttgcagggcttttgcgggaagtgataacggacgttgtacaagcgttcaggctcgcgtgcgcttgacaccgctagggctacagggaaatggacgatgccccgtaaagtggcattttcccttaccggagatgggatcggcggctcttcgctggcctaaccggaaactccacagctcgcgcgctcaccttaagcattggccartgaaacgcgcgcctcggcgatttagcttcactgctartgcccggcatgcktaggcgcgtgaggtcgcggtggggtgttgtcgcggagagtccgaggatgtgagttagagcaacggcaggagcgcgtgcgcacaggttaggcgtcacccggataggccgggtcgcgctggtgtgctccagatagattcggtccagcacgtcttgacctatgaagctgctttgggaagccgcgacgttggtagctttgaccaatcggttccagaagcgatcgcagtggaaaaagtgttctgagggcagtgacctaaggctctatgttagcaagagtccttttactttctgcttgatttcaactggacactctttaaagccgtatttcccggcattctctttagatgagttaaatcccagatagtagccatcgtaacaaaaatagtaattgtcaatctttcgcgtgcttggcttgtctttttccagcatgtctagaagcgtctttaattgtgagcgcgttagccaaaaattttgaggtacagcagagttgcattcatatgttcagctgtaagagtaacctagccataaagtttctttaacattcgcagtcaggtcacgcacgagaatcgttcgacttgccgaaaccatagcgtgccaggaacttggtacggccgagctgcacgaattcatcgatggcagcctgcacggcggcggggctacggagctgggaaagctgggacaaaaacgaccacgtccttatgttgtgtaacgccgggaaccctcaagctgcagctttctcgtttcgcaggccctggcccgatggttgcgggccgggccgcacttacatcggccttacgctcaagtatgatcggcaaaccctgccgccaccaaggacaacctcgtgccgatcgatgcctcgccgccctcctcgctcgttgatgtagcgccactggccgaagcgctcgaacagttcgccgaggcgcgcaactgggcgcagttccactcgccaaagaacctggccatggcccttgcgggcgagacgggcgaactcctcgagatccgtcgacctgcagccaagc

SEQ ID NO: 14 - DNA, 1173 bp, Pseudomonas stutzeri
  CDS (1)..(1173): ORF F
atgccactatcagcgctggtggcgccggctggggaactgagccgcgccgagatcaaccgttacagccgccacctactgatacccgatgtgggcatgatcgggcagcgtcggttgaagaacgccaaggtgttggtcatcggcgccggcggtcttggctctccgactctgctctatctagctgcagcaggtgtgggcaccatcgggataatcgactttgatcgggttgacgactccaaccttcagcgccaggtcatccacggggtggataccgtgggcgagctcaaggtggacagtgcaaagaaagccattgcgcgactaaatccctttgtccaggtcgaaacctataccgatcgcctggaacgggacatggcgatcgagctgttttcgcgctacgacctgatcatggacggtaccgacaacttcgcaacccgttacttggtcaacgacgcctgcgtgctggccaacaaaccctatgtgtggggctcgatattccgtttcgaagggcaggcgtccgtgttctgggaaaacgccccgaacgacctgggcctgaactaccgcgacctgtatccggagcctccgccgcccgagatggccccctcgtgctccgagggcggtgtgttcggcattctttgcgcatccatcgcatcgatcatggccaccgaggcggtcaagctgatcacgggcatcggcgagccactactgggtcggctggtggtgtacgacgctttggatatgcgctatcgggagcttcctgtgcgccgcctgccaaatcgacaaccgatcaccgacctggccgaggactatcaggtgttctgcggtctggggttgcccaaaggtgacacggcggacgccgtgccagggatcagcgtaacggaactcaagaagcggatggatcaggacgaggtgcctgtgctgatagacgtgcgcgagcccaccgagtgggacatcgtccgtattccgggcgcaatcttggtgaccaaatcgcccaccgcagcgcagacactgcgcgagcgatacggggcagatgccaacctggtgatcgtctgcaagtccgggcggcgctctgccgacgtgaccgccgagttgctaaacctgggcatgcgcaatgttcgcaacctcgaaggtggcgttttggcctgggtgaaggacgtggactcttctctgcctagctac

SEQ ID NO: 15 - protein, 391 aa, Pseudomonas stutzeri
MPLSALVAPAGELSRAEINRYSRHLLIPDVGMIGQRRLKNAKVLVIGAGGLGSPTLLYLAAAGVGTIGIIDFDRVDDSNLQRQVIHGVDTVGELKVDSAKKAIARLNPFVQVETYTDRLERDMAIELFSRYDLIMDGTDNFATRYLVNDACVLANKPYVWGSIFRFEGQASVFWENAPNDLGLNYRDLYPEPPPPEMAPSCSEGGVFGILCASIASIMATEAVKLITGIGEPLLGRLVVYDALDMRYRELPVRRLPNRQPITDLAEDYQVFCGLGLPKGDTADAVPGISVTELKKRMDQDEVPVLIDVREPTEWDIVRIPGAILVTKSPTAAQTLRERYGADANLVIVCKSGRRSADVTAELLNLGMRNVRNLEGGVLAWVKDVDSSLPSY

SEQ ID NO: 16 - DNA, 1743 bp, Pseudomonas stutzeri
  CDS (1)..(1743): ORF J
atgcctcactcgcaagtcttacttctctgtcaacgccccctggaggttcctatgaacaacgctggcatcatcgacctggttcctgctgaggaacgccaacgttgggtgcaggacggtacctacccgaaccagcccgtattcacgctgtttgccgccaaagccgaagcgcatcccgacaagaaggccgtgctgtcgccgcaaggtgacgtgacctacggcgagctcctcgatgcagccctgcggatggctcacagcctgcgtgattcggggatcgtggccggcgacgtggtggcttaccagctcaccaaccactggttgtgctgcgcaatcgacctggcagtggcagcgctcggtgccatcgtcgcccccttccctccgggacgcggcaagctggatatccaatcgctggttcgccgctgcgacgcgcgagcggtgatcgtcccgcaagcgtacgaaggcatcgatctgtgcgaggttatcgagtcactgcgccccaccctgctatccatgcgccgcctgattgttcagggcaagcctcgcgaaggatggattacgctcgatgagctgatgagcaccgagccgctggatctcgccagcctacccagggtgtgcccgaactcgccggtgcgtctgctggtgtcttcaggcaccgagtcggagcccaagctggtggcgtactcgcacaatgcgttggttggtggtcgcgggcgcttcctgcagcgcatcgcgtccgatggcgaagattttcgcggcatgtacctcgttccgctgggttcgtccttcggctccactgccaccttcggtgtgttgtgctggctgggtggttcgctggtcgtattgcccaagttcgacgtggatgaagccatcaaggcgattgcggcatttcggccgggcttcattctcggcgtacccaccatgctgcaacgcatcgccgctcaaccggcgttggagagcatcgacaaatccagcctgcgtggtttgatcgtcggcggctcggtcatcgacgaggccaccgtgcgcaaatgccgtgatgcgtttggctgcggcttcatcagcctctacggttccgccgacggcgtgaactgccataacaccctggacgaccccatcgaagttgtgctgaccagcgtcggcaagcccaatccggcggtctgcgcgattcgtctggtggacgacgaaggccgggaggtccggcaaggcgaggttggcgaaatcaccgcccgcgggccattgactccaatgcagtacgtcaacgcgccggagctggacgagcgttaccgcgacccgcaaggctgggtgaagaccggagatctgggctacatcaacgacaagggttatctggtcctagccggtcgcaagaaggacgtcatcatccgtgggggcgccaatatcagcccgacccagattgaaggcctggtcatggcgcatcccgatgtcgtgaccgttgcgtgcattcctgttcccgatgatgatctcgggcagcgggtgtgcctttgcgtcaccttgcgcgagggtgcagcgaagttttccctgaaagcgatcaccgacttcctgcgcgaactgggactggaggtgaacaagctccccgagtacctacgcttctaccgcgctctgcctctgacaccggcgggaaagatcgataaaaaagcgctaaccgaggaagcccgcgagctgggcacctcgggcatttgtcccgctgggccggggcagtcgactcccgagcgcagcttacgggagtacgca

SEQ ID NO: 17 - protein, 581 aa, Pseudomonas stutzeri
MPHSQVLLLCQRPLEVPMNNAGIIDLVPAEERQRWVQDGTYPNQPVFTLFAAKAEAHPDKKAVLSPQGDVTYGELLDAALRMAHSLRDSGIVAGDVVAYQLTNHWLCCAIDLAVAALGAIVAPFPPGRGKLDIQSLVRRCDARAVIVPQAYEGIDLCEVIESLRPTLLSMRRLIVQGKPREGWITLDELMSTEPLDLASLPRVCPNSPVRLLVSSGTESEPKLVAYSHNALVGGRGRFLQRIASDGEDFRGMYLVPLGSSFGSTATFGVLCWLGGSLVVLPKFDVDEAIKAIAAFRPGFILGVPTMLQRIAAQPALESIDKSSLRGLIVGGSVIDEATVRKCRDAFGCGFISLYGSADGVNCHNTLDDPIEVVLTSVGKPNPAVCAIRLVDDEGREVRQGEVGEITARGPLTPMQYVNAPELDERYRDPQGWVKTGDLGYINDKGYLVLAGRKKDVIIRGGANISPTQIEGLVMAHPDVVTVACIPVPDDDLGQRVCLCVTLREGAAKFSLKAITDFLRELGLEVNKLPEYLRFYRALPLTPAGKIDKKALTEEARELGTSGICPAGPGQSTPERSLREYA

SEQ ID NO: 18 - DNA, 1884 bp, Pseudomonas stutzeri
  CDS (1)..(1884): ORF I
ctggcgattcaaatccaggggcaagtgcaacgcttttgtctctcggttttgaatatgccgacactcttaaatgaattttccctgctgcattcatccacttcgtttccgccgaattggaatgaactgcaacttagcctgacggaacaggccagattattgggcatttgcccgctcgcaatctcgccgcctgtggatatggaaggagccgcattccagctgcagcatccggctatttctcctattcaggcccacttcgcctcaccagccggctggctgccaaatcgacacctctcggagctgctgctgcaggcgggcagcggtcttatgtcggtgcacggccgtgctagcggtagggcccaaccgctgggcgtggattatctttcgacacttaccgccgtcatgacgctgcacggaacgctggccgcagccgtggggcagctgcgtggcggtgcatttgatcaggttcagctttctccactgggatgcgggctgctcagtatcgggcagtatctggcaggcgccacggcaccagaagatcgtgaggcgttcctgccgggcggctccgatccgcatttgaggccgccatttcgttccgctgacggcatcacattcgagctggaaacgctcgacagcacaccgtgkcgaagcttttggaccgccgtcggcattgaatcggaattggccggtacggcctggaaaggttttctgcttcgctacgcgagggccgtgtcgcctctacctgccgcctgtctcacggcgctcgcccgcctgcgttacgcaaagatccaacaattggcagcgcaagcgggtgttgcggtcgtgcccgtccgcaccgatgcgcaacgccgcgaggaccccgattaccggcagtcactggctacgccatggcagttcgagtctttcccgccgtcccccgaaaggcatcgagacaccgcatttccgtcactgctgccgctacaggggatgcgcgtcatcgaatcctgtcgacgcattcagggaccgctggccgggcatctgctggcatcgctgggcgccgaagtcattcggctggagccgccgggtggcgatccgttgcgagccatgccgccctgcgccgaaggctgttcggtgcgctttgacgcgctgaaccacctcaaatccgttcacgaagtcgatatcaaatccgcccatgggcggcagttggtctacgagctcgcccgcgatgcggatgtctttctgcacaactgggcgcccggcaaggcccatgaaatgcaactggatgctgaacatctgcgcagggttcaaccacatctcgtttacgcctatgcgggaggctggggccgggctcccgtcaatgccccgggtaccgacttcaccgtccaggcctggtcgggtgtgtccgccgccattgcacgtcaatccggcatccgcggcggctcgctgttcaccgtgttggatgtgctgggcggcgcgatcgcggcactgggtgtgacggccgcgttgctcaatcgagcagtcacgggcacgggtacttatgtcgagagctcattgctgggcgccgccgatctgctgatgcacagcagcggcaaggcgtcgaggggcatcttgtccggcgtgtatcccacgctatcgggactgatcgccatcgactgccaacacccagatcagttccagtcgctggccatgttgctggacattcctgccactgcggatacctgccagcagacgctggcggagcgcttacgcaagcgacccgcttcggaatgggaaacggtgctgaacgaacggggcatcggcgcctgtgtagtcatcgaagacctcaagcagctcgccgccgacacccgcatctctgaatgcctcactcgcaagtcttacttctctgtcaacgccccctggaggttccta

SEQ ID NO: 19 - protein, 628 aa, Pseudomonas stutzeri
  misc_feature 214: Xaa = unknown amino acid
LAIQIQGQVQRFCLSVLNMPTLLNEFSLLHSSTSFPPNWNELQLSLTEQARLLGICPLAISPPVDMEGAAFQLQHPAISPIQAHFASPAGWLPNRHLSELLLQAGSGLMSVHGRASGRAQPLGVDYLSTLTAVMTLHGTLAAAVGQLRGGAFDQVQLSPLGCGLLSIGQYLAGATAPEDREAFLPGGSDPHLRPPFRSADGITFELETLDSTPXRSFWTAVGIESELAGTAWKGFLLRYARAVSPLPAACLTALARLRYAKIQQLAAQAGVAVVPVRTDAQRREDPDYRQSLATPWQFESFPPSPERHRDTAFPSLLPLQGMRVIESCRRIQGPLAGHLLASLGAEVIRLEPPGGDPLRAMPPCAEGCSVRFDALNHLKSVHEVDIKSAHGRQLVYELARDADVFLHNWAPGKAHEMQLDAEHLRRVQPHLVYAYAGGWGRAPVNAPGTDFTVQAWSGVSAAIARQSGIRGGSLFTVLDVLGGAIAALGVTAALLNRAVTGTGTYVESSLLGAADLLMHSSGKASRGILSGVYPTLSGLIAIDCQHPDQFQSLAMLLDIPATADTCQQTLAERLRKRPASEWETVLNERGIGACVVIEDLKQLAADTRISECLTRKSYFSVNAPWRFL

SEQ ID NO: 20 - DNA, 357 bp, Pseudomonas stutzeri
  CDS (1)..(357): ORF A
gatctgatcatctgcgagggacgcaatctgcacccggaggacatcgagcacactgtgatcgaggcgctgagcgacttgcgggcgcaaagctgcgcagtgttcagccatgacgacgaccagcagcgccagaccatcgtcgcagccatcgaactgaatcgtgaactcaagcgccgcctgcaggacaattgccgccagctcaaggccgtggtgcgtagcgcggtggtggacagtcatggcatcaccctcaaccgcatcgtcttcgtgcagccgaccagcattcacaagacgaccagcggcaaaatccagcgcgcgaagatgcgccagctctatctcgccgaggaactggacatgctgcaa

SEQ ID NO: 21 - protein, 119 aa, Pseudomonas stutzeri
DLIICEGRNLHPEDIEHTVIEALSDLRAQSCAVFSHDDDQQRQTIVAAIELNRELKRRLQDNCRQLKAVVRSAVVDSHGITLNRIVFVQPTSIHKTTSGKIQRAKMRQLYLAEELDMLQ

SEQ ID NO: 22 - DNA, 939 bp, Pseudomonas stutzeri
  CDS (1)..(939): ORF B
gtgaactcaagcgccgcctgcaggacaattgccgccagctcaaggccgtggtgcgtagcgcggtggtggacagtcatggcatcaccctcaaccgcatcgtcttcgtgcagccgaccagcattcacaagacgaccagcggcaaaatccagcgcgcgaagatgcgccagctctatctcgccgaggaactggacatgctgcaatgaaacccggtgccccttcagccctggtgcagtggctggccgatgcggcggcaccagtgagcctggtctgtttccattgcgccggcggcagcgcgcaagagttttttcccgtggaaaaaggccgcccaaggcctttgcgagctgtatgccgtcgaactgcccggccgcagctcggcgcttgcgcgagccgttcgccgagtcgctggcgcaactggccgaggccttcgccgagcagtgtcgcgccctgccgaacaaaccgcttatcctcttcggtcatagcctgggcgcactgctcgcatatgaaaccgctcgtgtgttgctggccaaaggcgaaaggccacccgtgcagctgctggtgtcctcgcgccagagcccggactggctgccggcctgcgcgggtctgccggcgctaaacgatcaggctctgcgcgattacctaggcaacctcgccggcaccccgccagaggtgctgcagagcaaggcgatgatggatctcgcggtgccggtgctgcaggccgacctgaagctgatcctcaattaccagcatcgacatctccagcccctgagtatcccggtgctggtcttcggcgcgatcagcgaccaacaggtgcgctatgagtcgctgctgagttgggagcggatcagcggcgaggggttcagcctgcggatgatcgagggagggcactttgcggtaatgcagcaaccgcagtgggtgcttgaccaggtgcaaaccgagtta

SEQ ID NO: 23 - protein, 313 aa, Pseudomonas stutzeri
VNSSAACRTIAASSRPWCVARWWTVMASPSTASSSCSRPAFTRRPAAKSSARRCASSISPRNWTCCNETRCPFSPGAVAGRCGGTSEPGLFPLRRRQRARVFSRGKRPPKAFASCMPSNCPAAARRLREPFAESLAQLAEAFAEQCRALPNKPLILFGHSLGALLAYETARVLLAKGERPPVQLLVSSRQSPDWLPACAGLPALNDQALRDYLGNLAGTPPEVLQSKAMMDLAVPVLQADLKLILNYQHRHLQPLSIPVLVFGAISDQQVRYESLLSWERISGEGFSLRMIEGGHFAVMQQPQWVLDQVQTEL

SEQ ID NO: 24 - DNA, 564 bp, Pseudomonas stutzeri
  CDS (1)..(564): ORF D
atgtcttacaggccaggcactgcacgcccgagaggaactggacgactacccacgaagccaaaccctgtcgaaacactgccgttcccttctctccttgctcgaccgcacgcgctgcaatcgtgttggccgacgcagctgaccgagccgctgcgcgatggcaggccgtgtccggtctcacccgctcctgctggacggcggcagggatgggccgataccgcccgggtgaagatggctcaggtgcaggcggtcagggccgacgtgtcctgtgagattcttcctcgacagcccgtgttttcgccgataggacggacactgaacatggacggcatgcccggcttggtggcggtcgcactggcgaagccgaagctggaagtgtccggtgtagggttcgagtgcccggccgccgccgtcgtggcgcagcagcccatcgaaagcgtcggacttcccgaccgtctgagtgcgcgcagcggcgatctggtacaggaggtcgagcaacgggatgacgtctgttttccgttcgcttccgtcgctggcctgctcgcccgcaaagaggaaaacctctca

SEQ ID NO: 25 - protein, 188 aa, Pseudomonas stutzeri
MSYRPGTARPRGTGRLPTKPNPVETLPFPSLLARPHALQSCWPTQLTEPLRDGRPCPVSPAPAGRRQGWADTARVKMAQVQAVRADVSCEILPRQPVFSPIGRTLNMDGMPGLVAVALAKPKLEVSGVGFECPAAAVVAQQPIESVGLPDRLSARSGDLVQEVEQRDDVCFPFASVAGLLARKEENLS

SEQ ID NO: 26 - DNA, 1113 bp, Pseudomonas stutzeri
  CDS (1)..(1113): ORF E
ttgttccagcttgccgcgctgccggcgatattcaaattcctgttggctgtgccggtgcagcgtgtgcgcctcgggcgcggacatttcgtgcactggttgctgttgctctgtgcgctactactggcgctgtactggctaatcggacggcataatctgatcggcgatcgcataatgctgttcgcgctgaccttcgccatcagcattgccgccacgtgggccgacattccgctaaatgcgctagcggtgcagtggttgccgcgtagtgaacagttgcgcgccggcagcatccgttccgcagcgctgttcgtaggcgccattgttggcggcggcgtcatgatcatggtgcaggcgcgcgtgggctggcaggcccccttctggctgctaggggtcggactgctgattggcgccctgcccttcctgctgttgcgtagacacgccgcactgcccgagcaggccgagccgcgcgagactacagatcctccaccgggcgtgatggcggactgggcaagcttcttccaccagccaggggcgcggcaatggacattgctgctgctgaccagtttccccttcctcggcgcgacgtggctgtacctcaaacctttattgttggacatgggcatgcagctagagcgcgtggccttcatcgttggcatcgtcggcggcaccgcaggcgcactgttcagcctgctcggcggacagctagtgcaaatgttgggcatagcacgggccattgcctggtacctgctggcggcgctgggcgcgctggcacttttgacgttcagcgtctgggcccaactgggggcggcatggctgattgccagcgccctctgcgtggcagccagcatgggcgccatctcggcgctgatgttcgggttgaccatgttcttcacccgaaatcggcgcaacgcgtcggactatgccctgcaaaccaccatgttcaccgttgcgcgactggcggtgccgatcgccgccggggtgttgctcgaccgggtgggctacaccggcatgctcctggcaatgaccctggcgctgctgctttccttcgcgctcgcctgtcgggtgcgggaaaaggtggaatcttcggcacagtcgatactcgagcacgagcgggtt

SEQ ID NO: 27 - protein, 371 aa, Pseudomonas stutzeri
LFQLAALPAIFKFLLAVPVQRVRLGRGHFVHWLLLLCALLLALYWLIGRHNLIGDRIMLFALTFAISIAATWADIPLNALAVQWLPRSEQLRAGSIRSAALFVGAIVGGGVMIMVQARVGWQAPFWLLGVGLLIGALPFLLLRRHAALPEQAEPRETTDPPPGVMADWASFFHQPGARQWTLLLLTSFPFLGATWLYLKPLLLDMGMQLERVAFIVGIVGGTAGALFSLLGGQLVQMLGIARAIAWYLLAALGALALLTFSVWAQLGAAWLIASALCVAASMGAISALMFGLTMFFTRNRRNASDYALQTTMFTVARLAVPIAAGVLLDRVGYTGMLLAMTLALLLSFALACRVREKVESSAQSILEHERV

SEQ ID NO: 28 - DNA, 2229 bp, Pseudomonas stutzeri
  CDS (1)..(2229): ORF L
atggcagcaagccaaagaggatgcgggcatatgcagcaagagagacggaatatcggaatactcttggtcagccaggatgaaaaactggcgttggacctggacatggtagtggaaagcgtaaacggtttgctcagtcgcaataccgatacgcctttcgatttgcacccgaacgacgaatgctttccctaccgccagatcttcgctcaggcctgtcgatacttgcgctcacaacccagggacaagctgcccgccctgttctctcggtgcttccactcaatggcaaccgcccgccaggcgctgtcggcgggtgattggacgttgtccccgcttgaatgcgtgttgctcgatacgcgccatgaggaaccggtcgatgacgaccccttccttgcgcttacgctgcaacaaccgccgacccggccctcgccctgctctgcaatgctgctgtgcgaggaaagggtgctgggtaaatggatgtgccggctggggggcaaccgcctggtgcgggtgccgcattcgaatccctggcaacgccgcgccgatatcctccggctgattctggatcatctggagcatgcccatttcaaccgcatgcttgctcgcgcgcgccagaaagccgatggccaggtgacgctggcacaaaagattcatcacctcatgactgaacgctggggtgatcaatgggacttccacttctacacgggctccatggtcgccggtttcatcgactcgatgaaatctctgctgcagggaacggacagccactgcctgaccggtaacaacgagcactcattggccgtaagcgcgctggctggctggcagttgtatggccgtgcctacgtcatcgccatgacctccgggatgatcgacgaagcgcgcgggactctggcgaatctcaagcgtgccgcagcccctggcatcattgtctgcgccgactccccggaaacgatctggtatccattccagggtacgctcgacgccgacagcgacggacatgcggtcattgcagcacgcggcttgtggcatgggttcatgcgtacccccgatgacatgccagcttgccttgcaaatgccttccaggcccttgatgagcgccccgctccaaccttcgtccttgcaacgcaacacgtgctggagtcgcaagcgaccatttcggaaccagtggtgcagcgcccctcctccaaggctgcaacgctctcctgcgcgcaacgcgaacgactggatcaggccgtggccgccatcaaccacgacaacgcccggatgctgtggcattgcggtcgcctcacatcggacgagcggaatcagattcttcgcctggccgaaaaagccggcatcgccctggtggacagcatcattcatccgggaagcgtgcccgggttcagcgacggcaagtccgtagcgaactatctgggaacgctttccatgtatggattcaaccgcgcggtctatgaattcctggaagcgcaaagcaccaacgaagaaggcgctccctggctctttttcctcaagggcaaggtagagcaatcgtctaccccgtattcggaaggcaagctcaagcgcaacttccgcattggccaagtcaactgcaatgaggcgcacctgtcgccattcaccctgctgggactggacgttcggttggcggacttcctgaattacctcgaaccacgcctgcaggttgacgacgcggtgctacggcagcgccgcgccagaatcgaacaattgcgcaagctgccggccgctcagccaagcgacctgatcgaaacaatgccgatgaccccgaattatttcttccaccagttgggacgactggtggtcgatctgatcgagtcgcgaagctaccgctataccggcgtctacgacgttggtcgttgcggtttgtcggcaatgcgcaacgtggcacgcaccgatcccggtttctccggctggtacggtcgcgccctgatgggtgacggtctgatgtcgctgccctatatcgccttgaagaacgaacgcaatgtgctggcattcatcggtgatggtgctcgcgcaatagtgccgaatgtggagcagcgcctggttggttctttcgatccggggcatcgcagggcgtggtggcaacgtcaccgttttctatctgagcaacggcgtgctttccatgattcagacctatctggacaaacgctacacgctcaatggctgcagccaggtcaacgtgccattgacggaatggaaggaggctcccgtcgagcatgc

SEQ ID NO: 29 - protein, 743 aa, Pseudomonas stutzeri
MAASQRGCGHMQQERRNIGILLVSQDEKLALDLDMVVESVNGLLSRNTDTPFDLHPNDECFPYRQIFAQACRYLRSQPRDKLPALFSRCFHSMATARQALSAGDWTLSPLECVLLDTRHEEPVDDDPFLALTLQQPPTRPSPCSAMLLCEERVLGKWMCRLGGNRLVRVPHSNPWQRRADILRLILDHLEHAHFNRMLARARQKADGQVTLAQKIHHLMTERWGDQWDFHFYTGSMVAGFIDSMKSLLQGTDSHCLTGNNEHSLAVSALAGWQLYGRAYVIAMTSGMIDEARGTLANLKRAAAPGIIVCADSPETIWYPFQGTLDADSDGHAVIAARGLWHGFMRTPDDMPACLANAFQALDERPAPTFVLATQHVLESQATISEPVVQRPSSKAATLSCAQRERLDQAVAAINHDNARMLWHCGRLTSDERNQILRLAEKAGIALVDSIIHPGSVPGFSDGKSVANYLGTLSMYGFNRAVYEFLEAQSTNEEGAPWLFFLKGKVEQSSTPYSEGKLKRNFRIGQVNCNEAHLSPFTLLGLDVRLADFLNYLEPRLQVDDAVLRQRRARIEQLRKLPAAQPSDLIETMPMTPNYFFHQLGRLVVDLIESRSYRYTGVYDVGRCGLSAMRNVARTDPGFSGWYGRALMGDGLMSLPYIALKNERNVLAFIGDGARAIVPNVEQRLVGSFDPGHRRAWWQRHRFLSEQRRAFHDSDLSGQTLHAQWLQPGQRAIDGMEGGSRRAC

SEQ ID NO: 30 - DNA, 2292 bp, Pseudomonas stutzeri
  CDS (1)..(2292): ORF M
atgttgagtcagccgaggcgcattgcggagcgtgtcgtcgaaggcgtcaatgcgttaaaggacgagggggcagaactcgtagggctgggaggcttcacgtccattgttggcaatcgcggcctgcagacactggatcgcacgcaaattccggtgacgacgggtaactcgctgactgcgtatgccgcctatatgaatctgttgggtgtcctatccgcgctggaaattccaccagaaaaagcggaggtcgccgtgctcgggtatcccgggtcgattgcactggccatcgtttgcctgctggcgccattggggtgtcggctgcgactggtgcatcgcggagggaaacagcaggtcgacacgttgcttgagtacctgccctcacaatttcacggacaagtgacgctgcacgccggtctggaagattgctatgcccaggtccgattgtttgttgccgcgacctccaccggtggggtcattgatcctcggcggctggcttgcggtagtgtcgtggtggatgcagcgttgccgaaggacatgcaacctggctgggaaaaacgcgacgacattctggtcattgatggcggcctggtctctgcgactgacgcagtggacttcggggccatggcgctgggcctgggtcctaagcgcaatatcaatggttgcctggccgaaaccatgatcctggcgctgcagggtcgcgcagaggcattctccatcgggcgcgaactgcctgctgagaaggttctcgagatcggtcgcatcgccgagggacatggctttctgccttaccccatggcgagcggcggcgaatcagtggatggcgctcggttcgatgaactgcgacgatttcacggtgccaggccgccagctgtcgtacacgacctgaataccggctcccaggagcttcgctcagaagtgctgcgctgcttcggtacgcacatcaaccccattctgcgtgaattctatgagttcaatcatgtcgagcgtgtcttcagcaatggccaaggctgctggctgacggatctggacggtcgccgttacctggatttcgtagcgggatacggctgcctcaacacgggccacaaccatccggagatagcagccaggctgcaggaatacctcacccagcagcatcccaccttcgtgcagtacctgtctgcgccgttgcatgccagcctgctggccaaacgtcttgccgagctggctccagccggactggagcgcgtgttccttagcaactctgggaccgaagccgtggaggcggctttgaagctggctttggccgccagcgacaaatccacgctgctctactgcaccaatggctatcacggcaaaacgcttggcgccctgtccgtaacggggcgtgagaagcaccgcaaggcgttcgaaccgttgctgccgcgctgcgaggagattccgttcgccgatgtttcggcgttgcgaaaccggttgctcaagggggatgtcgcamccttcatcatggaaccgatccagggtgaaggtggtgtcaccatggccccggacggctatctcagagttgtgagggacctgtgctccgagcatgaatgcctctggattctcgatgaaatccagaccgggctcggacgtaccggcaagatgttcgcctgcgaatgggaagacgtctcgcccgacatcatcgtgctatccaaatccctgtcgggtggtctggtgcctatcggcgcaacgctgtcctcgaaagaagtctggcaacgcgcctacggcaatatcgaccgattcgcattgcacacctcgacgtttggcggcgggaattttgctgccgccgccgccatggccgcgctggacgtgatcgagcacgaagacctgcccggcaatgccgctctggttggtgcacacctgcgacaagggctggaggcgctggcccgcaagcactatttcatcaaggaggttcgcgggcgaggcctgatgatcgccatcgaatttcagaacgatgtctcaaatggcattgaagccttcgtgcgggatatgaccagccggatgcccgccaatgccgcagccacctaccgaatgatgcctgccaaggcccgcgaacacctcgaagcagccatgcgcgaattggagtccacgctggccgacatgttcgtcctgcgcatcatgaccaagttgtcccaggagcacggcatcctgaccttcgtgacggccaacaacaaccgtgtcatgcggattcagccgccgctggttctatcactggcagaagccgatcggttcataaaggcattgggcgaggtctgcgaggatctctcgacctttgagtca

SEQ ID NO: 31 - protein, 764 aa, Pseudomonas stutzeri
  misc_feature 490: Xaa = unknown amino acid
MLSQPRRIAERVVEGVNALKDEGAELVGLGGFTSIVGNRGLQTLDRTQIPVTTGNSLTAYAAYMNLLGVLSALEIPPEKAEVAVLGYPGSIALAIVCLLAPLGCRLRLVHRGGKQQVDTLLEYLPSQFHGQVTLHAGLEDCYAQVRLFVAATSTGGVIDPRRLACGSVVVDAALPKDMQPGWEKRDDILVIDGGLVSATDAVDFGAMALGLGPKRNINGCLAETMILALQGRAEAFSIGRELPAEKVLEIGRIAEGHGFLPYPMASGGESVDGARFDELRRFHGARPPAVVHDLNTGSQELRSEVLRCFGTHINPILREFYEFNHVERVFSNGQGCWLTDLDGRRYLDFVAGYGCLNTGHNHPEIAARLQEYLTQQHPTFVQYLSAPLHASLLAKRLAELAPAGLERVFLSNSGTEAVEAALKLALAASDKSTLLYCTNGYHGKTLGALSVTGREKHRKAFEPLLPRCEEIPFADVSALRNRLLKGDVAXFIMEPIQGEGGVTMAPDGYLRVVRDLCSEHECLWILDEIQTGLGRTGKMFACEWEDVSPDIIVLSKSLSGGLVPIGATLSSKEVWQRAYGNIDRFALHTSTFGGGNFAAAAAMAALDVIEHEDLPGNAALVGAHLRQGLEALARKHYFIKEVRGRGLMIAIEFQNDVSNGIEAFVRDMTSRMPANAAATYRMMPAKAREHLEAAMRELESTLADMFVLRIMTKLSQEHGILTFVTANNNRVMRIQPPLVLSLAEADRFIKALGEVCEDLSTFES

SEQ ID NO: 32 - DNA, 1539 bp, Pseudomonas stutzeri
  CDS (1)..(1539): ORF O
gtggccaccatgtctggcaccagggagaactccccgaggcgttgttcgccgcaggattcgctaacgtcgccaccttcgagagcgacttgtttccattggcgccagtgcaagtgctggtgtgccgatgatgtctttggccctcggactagccgatggagccgatctggaaaagcaggccgactccgcgttcattgccatcatccgcagcgagcaacgtgtgcgcgacgaaaccgtaggcaggtctttgtgtcgtgtcacctacggcatctgctacaccacccgcacaagcgtcgccgagcacgtggtgttcccttgggccagttaccgcctcgctggcaacttgatccatcaggccggctttcacgcttggccggaacgccggaagccaacgacatgatcgatttttcccttcccaatgaagtgcaaatgctggtttcgacggtgaagcggttcgtcgaaaacgaactcaatccgctcgaggatgagatcgaaagaacgaatgcgatcgatcccagcgtggctgaaggcctcaagcaaaaggcccgggagctggggctgtgggccatgcatatgccgcaggaggtgggcggcggcgggctgagtgccgttgaattctgtctggtcaatgagcagatcggccgaaccaaggatgtgttggcaaggcgggcattcggccacgttcccagcatccttgttcattgtaccggcgagcagcgcgagaagtacctgcatgccgccatgcgtggcgatatacatgtgtcggttgccatgagcgagcccgaggccggttcggacgcaaacggcatcagaaccgcggtcaagcgcgacggttgcgagtggatattgaacggcagcaaacacttcatcagcgatgccgatatcgcgagcgcttatatcgtcactgcacgcagcgaggaaggtatctcctgctttttggtcgaccgcgatacgccgggccttgaactcgggccgatacaggaaatgatggggcatcgcggtacccatcagcacgggctgttcttcaccgactgccggatcgcgccgcaacaattgctcggcgagccggggcggggcatgtcgctggtacttggccatctcaacgttgcacggttggcctatgtcggtgccagggctgtcggcatggcttcgaaactactggaaatgtcggtcgatttcgcgaagcagcgctcgcagttcggtgccccgatcggcagtttccagatggtgcagaagatgctggcggacatgcagtgtgaaatctacggcgccaggatgatgcttctcaacgcggcctgggaaatcgatcaagggcgggatgtgcgtgagaaggtttcgatggtgaagttgttcgcgtccgaaatgctgggccgcgtcgcggacagcgcagtgcagatctttggcggaatgggctattgcactgagttgccgatcgagcgctactaccgcgatgcgagggtcttccggctttacgatggtacctcggaaattcatcggatcatgatcgcgcgccgcctgctcgaaagaggaatttcgctgctt

SEQ ID NO: 33 - protein, 513 aa, Pseudomonas stutzeri
VATMSGTRENSPRRCSPQDSLTSPPSRATCFHWRQCKCWCADDVFGPRTSRWSRSGKAGRLRVHCHHPQRATCARRNRRQVFVSCHLRHLLHHPHKRRRARGVPLGQLPPRWQLDPSGRLSRLAGTPEANDMIDFSLPNEVQMLVSTVKRFVENELNPLEDEIERTNAIDPSVAEGLKQKARELGLWAMHMPQEVGGGGLSAVEFCLVNEQIGRTKDVLARRAFGHVPSILVHCTGEQREKYLHAAMRGDIHVSVAMSEPEAGSDANGIRTAVKRDGCEWILNGSKHFISDADIASAYIVTARSEEGISCFLVDRDTPGLELGPIQEMMGHRGTHQHGLFFTDCRIAPQQLLGEPGRGMSLVLGHLNVARLAYVGARAVGMASKLLEMSVDFAKQRSQFGAPIGSFQMVQKMLADMQCEIYGARMMLLNAAWEIDQGRDVREKVSMVKLFASEMLGRVADSAVQIFGGMGYCTELPIERYYRDARVFRLYDGTSEIHRIMIARRLLERGISLL

SEQ ID NO: 34 - DNA, 159 bp, Pseudomonas stutzeri
  CDS (1)..(159): ORF Q
gtgccgatcgatgcctcgccgccctcctcgctcgttgatgtagcgccactggccgaagcgctcgaacagttcgccgaggcgcgcaactgggcgcagttccactcgccaaagaacctggccatggcccttgcgggcgagacgggcgaactcctcgagatc

SEQ ID NO: 35 - protein, 53 aa, Pseudomonas stutzeri
VPIDASPPSSLVDVAPLAEALEQFAEARNWAQFHSPKNLAMALAGETGELLEI

(19) United States
(12) Patent Application Publication
(10) Pub. No.: US 2003/0135032 A1
(43) Pub. Date: Jul. 17, 2003

(54) Methods and compositions for bioremediation

(76) Inventors: Thomas A. Lewis, Milton, VT (US); Andrzej Paszczynski, Moscow, ID (US); Ronald L. Crawford, Moscow, ID (US); Jonathan L. Sebat, Pullman, WA (US); Marc S. Cortese, Moscow, ID (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/181,319

(22) PCT Filed: Jan. 19, 2001
(86) PCT No.: PCT/US01/02386

(51) Int. Cl. C07H 21/02; C07H 21/04; C12N 1/20; C12N 15/00

(52) U.S. Cl. 536/23.1; 435/252.3; 435/320.1

(57) ABSTRACT

The present invention provides isolated nucleic acid molecules that encode one or more of the enzymes required to produce PDTC. The present invention also provides isolated proteins encoded by nucleic acid molecules of the invention. In another aspect, the present invention provides methods for reducing the amount of a metal in a substrate, such as soil. In yet another aspect, the present invention provides methods for reducing the amount of carbon tetrachloride in a substrate, such as soil.

### FIELD OF THE INVENTION

This invention relates to nucleic acid molecules that encode enzymes required for the biosynthesis of pyridine-2,6-bis(thiocarboxylate), and to environmental remediation methods for removing metals and carbon tetrachloride from a contaminated substrate, such as soil or water.

### BACKGROUND OF THE INVENTION

Pollution of the environment is a major problem. Examples of environmental pollutants are metals (including radioactive metal isotopes) and organic molecules, such as carbon tetrachloride, that are produced as by-products from numerous industrial processes. One approach to removing pollutants from the environment is bioremediation which utilizes one or more biological organisms to physically remove and/or degrade environmental pollutants. Ex situ bioremediation techniques involve the removal of the contaminated substance, such as contaminated soil, to a treatment facility where the contaminant(s) is removed. In situ bioremediation does not require the physical removal of the contaminated substance, which is treated at the site of contamination, and so is typically cheaper than ex situ bioremediation.

The present inventors have identified and isolated a portion of the Pseudomonas stutzeri genome that encodes enzymes necessary to synthesize pyridine-2,6-bis(thiocarboxylate) (abbreviated as PDTC). PDTC chelates numerous metal ions, and the complex formed between PDTC and Cu(II) ions is capable of degrading carbon tetrachloride. This carbon tetrachloride degradation ability is unique and particularly valuable since it mediates the conversion of carbon tetrachloride to non-toxic carbon dioxide. Other known bacterial carbon tetrachloride degradation processes convert carbon tetrachloride to toxic intermediates like chloroform.

Thus, as set forth more fully herein, the present invention provides compositions and methods that are useful to degrade carbon tetrachloride in substances, such as soil or water, that are contaminated with carbon tetrachloride. The present invention also provides methods and compositions that are useful to remove metal ions from (or immobilize metal ions within) substances, such as soil or water, that are contaminated with metal ions. The methods and compositions of the invention can be utilized in bioremediation.

### SUMMARY OF THE INVENTION

In accordance with the foregoing, the present invention provides isolated nucleic acid molecules that encode one, or more, or all, of the enzymes (or functional fragments thereof) required to produce PDTC. The present invention also provides isolated proteins encoded by nucleic acid molecules of the invention. Representative examples of isolated nucleic acid molecules and isolated proteins of the invention include the nucleic acid molecule consisting of the sequence set forth in SEQ ID NO:1 (encoding the protein consisting of the amino acid sequence set forth in SEQ ID NO:2), the nucleic acid molecule consisting of the sequence set forth in SEQ ID NO:3 (encoding the protein consisting of the amino acid sequence set forth in SEQ ID NO:4), the nucleic acid molecule consisting of the sequence set forth in SEQ ID NO:5 (encoding the protein consisting of the amino acid sequence set forth in SEQ ID NO:6), and the nucleic acid molecule consisting of the sequence set forth in SEQ ID NO:7 (encoding the protein consisting of the amino acid sequence set forth in SEQ ID NO:8). Thus, in one aspect, the present invention provides isolated nucleic acid molecules that are at least 70% identical to a nucleic acid molecule selected from the group consisting of SEQ ID NO:1, SEQ ID NO:3, SEQ ID NO:5, and SEQ ID NO:7.

In another aspect, the present invention provides isolated nucleic acid molecules that comprise a PDTC gene cluster as defined herein. The isolated nucleic acid molecules of the invention that comprise a PDTC gene cluster may optionally further comprise: a nucleic acid sequence that is at least 70% identical to the nucleic acid sequence set forth in SEQ ID NO:7; a nucleic acid sequence that is at least 70% identical to the nucleic acid sequence set forth in SEQ ID NO:9 (the nucleic acid sequence set forth in SEQ ID NO:9 encodes the protein consisting of the amino acid sequence set forth in SEQ ID NO:10); and a nucleic acid sequence that is at least 70% identical to the nucleic acid sequence set forth in SEQ ID NO:11 (the nucleic acid sequence set forth in SEQ ID NO:11 encodes the protein consisting of the amino acid sequence set forth in SEQ ID NO:12).

In another aspect, the present invention provides isolated nucleic acid molecules that are at least 70% identical (such as at least 80% identical, or at least 90% identical) to the nucleic acid molecule consisting of the nucleic acid sequence set forth in SEQ ID NO:13 which is a portion of the Pseudomonas stutzeri genome that encodes all of the enzymes necessary to synthesize pyridine-2,6-bis(thiocarboxylate). The present invention also provides vectors that include one or more nucleic acid molecules of the invention, and host cells (including bacterial and plant cells) that include one or more vectors of the invention.

In another aspect, the present invention provides isolated proteins, such as isolated proteins that are at least 70% identical to one or more of the proteins consisting of the amino acid sequences set forth in SEQ ID NO:2, SEQ ID NO:4, SEQ ID NO:6, and SEQ ID NO:8.

In another aspect, the present invention provides methods for reducing the amount of a metal in a substrate, such as soil or water. In yet another aspect, the present invention provides methods for reducing the amount of carbon tetrachloride in a substrate, such as soil or water.

In yet another aspect, the present invention provides methods for immobilizing metal ions within a substrate (such as soil), the methods including the steps of: (a) contacting PDTC with a substrate to form a metal complex with the metal ion species; and (b) allowing PDTC to form a metal complex with the metal ion species thereby immobilizing the metal ion species. The methods of this aspect of the invention are useful, for example, for immobilizing metal ions in contaminated soil. The PDTC forms a complex with the metal ions to form a water-insoluble complex, or a complex which diffuses through the soil more slowly than uncomplexed metal ions.

The nucleic acid molecules of the invention can be used, for example, to genetically modify an organism (such as a microorganism or plant), to confer on the modified organism the ability to synthesize PDTC (or augment the existing ability of the organism to synthesize PDTC). The compositions and methods of the present invention are therefor useful, for example, in bioremediation, such as in situ bioremediation where one or more biological organisms are genetically modified to gain or augment the ability to remove metal ions from the environment, and/or to degrade carbon tetrachloride. In one representative example, the compositions and methods of the invention are useful to leach metals from metal ore. Some nucleic acid molecules of the invention (such as the nucleic acid molecule consisting of the sequence set forth in SEQ ID NO:13) are useful, for example, as vectors for the transfer of valuable genetic traits between different strains of bacteria. Further, the nucleic acid molecules of the invention can be used, for example, as probes to identify related nucleic acid molecules, or to inhibit the expression (such as through antisense inhibition) of related nucleic acid molecules. The proteins of the invention are useful, for example, to enhance or otherwise modify the biosynthetic pathway in microorganisms that results in the production or inhibition of PDTC.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a map of the insert of cosmid pT31 (SEQ ID NO:13) and positions of deletions and insertions used to assess the complementation of a PDTC phenotype. Open reading frames are indicated by arrows with letter designations below; the direction of the arrows indicate the direction of transcription/translation of the putative gene. Transposon insertions are represented by vertical lines with flags. The orientation of flags indicates the orientation of the lacZ or phoA gene of the transposon. Open flags indicate insertions with neutral effect on PDTC production; filled flags indicate inserts that have an effect on PDTC production (see Table 2). The column labeled ctt lists the CCl₄ transformation activity in units of μg CCl₄ ml⁻¹ day⁻¹ for each CTN1 transconjugant containing the respective cosmid. N.D.=not detected;

FIG. 2A illustrates the chemical structure of PDTC;

FIGS. 2B-2E illustrate the chemical structures of representative PDTC metal complexes;

FIG. 3 illustrates CCl₄ transformation by PDTC at different copper concentrations. Replicate reactions were started by addition of CCl₄, and individual reactions were sacrificed at the indicated time points. A. no added reductant. Symbols: □, no added copper (2 separate experiments); ▾, 0.19 μM CuCl₂; ♦, 3 μM CuCl₂; ▪, 5 μM CuCl₂; ●, 13 μM CuCl₂. B. 0.5 mM sulfide (added as H₂S). Symbols: □, no added copper; ♦, 7.5 nM CuCl₂; ▪, 75 nM CuCl₂; ●, 13 μM CuCl₂; +, no PDTC;

FIG. 4 illustrates reaction pathway explaining products of reaction between Cu[II]:PDTC and Cl₄. Compounds shown in gray are those that have not been identified;

FIG. 5 illustrates products of reaction between Cu:PDTC and CCl₄ detected by negative ion electrospray mass spectrometry. Reactions were conducted in DMF:H₂O using 2 mM Cu:PDTC and excess CCl₄. A. Whole reaction mixture after a 2-hour incubation. B. No CCl₄ added;

FIG. 6 illustrates the EPR spectrum of PBN-trapped radicals produced in reaction of Cu:PDTC and excess ¹³CCl₄. Reactions were conducted in phosphate-buffered aqueous solution with 5 mM PDTC, 50 μM CuCl₂ and 50 μM Na₄EDTA (included to aid solubility of the Cu complex), and 100 mM PBN. A. complete reaction mixture; B. mixture lacking Cu; C. mixture lacking PDTC;

FIG. 7 illustrates the positive ion electrospray MS/MS of products of reaction between 2 mM Cu:PDTC and excess CCl₄ in the presence of 2,2,6,6-tetramethylpiperidinyl oxide (TEMPO). A. ES+/MS spectrum of the reaction mixture in DMF:water (1:1, vol/vol). All ions except those at m/z140 and m/z142 were present in control incubations without CCl₄. DMF: N,N-dimethylformamide; TBA+: tetrabutylammonium cation. B. ES+ MS/MS daughter ion fragments from ion at m/z 142 from reaction in A. C. Daughter ion fragments from ion at m/z 142 from authentic 2,2,6,6-tetramethylpiperidine. For secondary ionization, argon gas was used;

FIG. 8 illustrates the potentiometric titration curve for pdtc titrated with a 1 N NaOH solution: [pdtc]=0.05 mM; T=25° C. and I=0.1M (NaClO₄);

FIG. 9 illustrates the stepwise protonation of pdtc;

FIG. 10 illustrates spectral changes during titration of pdtc by NaOH. I (ionic strength adjustment)=0.1 N NaClO₄: T=25.0° C.; l (path length)=1.0 cm; [pdtc]=0.275; and mM [KOH]=1.0 M: pH=(1) 1.00; (2) 1.21; (3) 1.51; (4) 2.08; (5) 2.98; (6) 4.11; (7) 4.75; (8) 5.00; (9) 5.35; (10) 5.63; (11) 5.96; (12) 6.82; (13) 12.5;

FIG. 11 illustrates the spectral changes during titration of Fe(pdtc)₂ by NaOH. I (ionic strength adjustment)=0.1 N NaClO₄: T=25.0° C.; l=1 cm; [PDTC]=0.275 mM; and [KOH]=1.0 M: (1) Free PDTC; pH=(2) 1.00 to 9.00; (3) 10.06; (4) 11.12; (5) 11.39; (6) 11.48; (7) 11.62; (8) 11.72; (9) 12.05;

FIG. 12 illustrates the graphical determination of inflection point of Fe(pdtc)₂ titrated by NaOH. I (ionic strength adjustment)=0.1 N NaClO₄; T=25.0° C.; l=1 cm; [pdtc]=0.275; and mM [KOH]=1.0 M, λ=350; and

FIG. 13 illustrates the spectra of various metal complexes and free pdtc. I=0.1 N NaClO₄: T=25.0° C.; l=1 cm; [M]=0.275 mM; and 2% HNO₃=1.0 M: (1) [FeIII(pdtc)₂]⁻¹⁻; (2) CoIII[(pdtc)₂]¹⁻; (3) NiIII[(pdtc)₂]¹⁻; (4) MnIII[(pdtc)₂]¹⁻; (5) CrIII[(pdtc)₂]¹⁻; (6) [pdtc]²⁻; (7) CuII[pdtc]¹⁻; (8) ZnII[pdtc]¹⁻.

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al. (1989) Molecular Cloning: A Laboratory Manual, 2^{nd} ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

As used in connection with the nucleic acid molecules and proteins of the invention, the term “isolated” means a nucleic acid molecule or protein that is substantially free from cellular components that are associated with the nucleic acid molecule or protein as it is found in nature. As used in this context, the term “substantially free from cellular components” means that the nucleic acid molecule or protein is purified to a purity level of greater than 80% (such as greater than 90%, greater than 95%, or greater than 99%). Moreover, the terms “isolated nucleic acid molecule” and “isolated protein” include nucleic acid molecules and proteins, respectively, which do not naturally occur, and have been produced by synthetic means. An isolated nucleic acid molecule or isolated protein generally resolves as a single, predominant, band by gel electrophoresis, and yields a nucleic acid sequence or amino acid sequence profile consistent with the presence of a predominant nucleic acid molecule or protein.

The term “percent identity” or “percent identical” when used in connection with the nucleic acid molecules and proteins of the present invention, is defined as the percentage of nucleic acid residues in a candidate nucleic acid sequence, or the percentage of amino acid residues in a candidate protein sequence, that are identical with a subject nucleic acid sequence (such as any one of the nucleic acid sequences set forth in SEQ ID NOS:1, 3 and 5) or protein sequence, after aligning the candidate and subject sequences to achieve the maximum percent identity, and not considering any nucleic acid residue substitutions as part of the nucleic acid sequence identity. When making the comparison, the candidate nucleic acid sequence or protein sequence (which may be a portion of a larger nucleic acid sequence or protein sequence) is the same length as the subject nucleic acid sequence or protein sequence, and no gaps are introduced into the candidate nucleic acid sequence or protein sequence in order to achieve the best alignment.

For example, if a 100 base pair subject nucleic acid sequence is aligned with a 100 base pair candidate portion of a larger DNA molecule (such as a genomic clone), and 80% of the nucleic acid residues in the 100 base pair candidate portion align with the identical nucleic acid residues in the 100 base pair subject nucleic acid sequence, then the 100 base pair candidate portion of the larger DNA molecule is 80% identical to the subject nucleic acid sequence.

Nucleic acid sequence identity can be determined in the following manner. The subject nucleic acid sequence is used to search a nucleic acid sequence database, such as the GenBank database (accessible at web site http://www.ncbi.nln.nih.gov/blast/), using the program BLASTM version 2.1 (based on Altschul et al., Nucleic Acids Research 25:3389-3402 (1997)). The program is used in the ungapped mode. Default filtering is used to remove sequence homologies due to regions of low complexity. The default parameters of BLASTM are utilized.

Amino acid sequence identity can be determined in the following manner. The subject protein sequence is used to search a protein sequence database, such as the GenBank database (accessible at web site http://www.ncbi.nln.nih.gov/blast/), using the BLASTP program. The program is used in the ungapped mode. Default filtering is used to remove sequence homologies due to regions of low complexity. The default parameters of BLASTP are utilized. Default filtering is used to remove sequence homologs due to region of low complexity.

The term “vector” refers to a nucleic acid molecule, usually double-stranded DNA, which may have inserted into it another nucleic acid molecule (the insert nucleic acid molecule) such as, but not limited to, a cDNA molecule or a fragment of genomic DNA. The vector is used to transport the insert nucleic acid molecule into a suitable host cell. A vector may contain the necessary elements that permit transcribing and translating the insert nucleic acid molecule into a polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated. Many molecules of the polypeptide (if any) encoded by the insert nucleic acid molecule can thus be rapidly synthesized.

The term “PDTC gene cluster” refers to a group of nucleic acid sequences that encode enzymes necessary to synthesize PDTC and that, when introduced into a host cell (such as a plant or bacterial cell), confer on the host cell the ability to synthesize PDTC. The PDTC gene cluster includes: (1) a nucleic acid sequence that is at least 70% identical to the nucleic acid sequence set forth in SEQ ID NO:1; (2) a nucleic acid sequence that is at least 70% identical to the nucleic acid sequence set forth in SEQ ID NO:3; (3) a nucleic acid sequence that is at least 70% identical to the nucleic acid sequence set forth in SEQ ID NO:5; (4) a nucleic acid sequence that is at least 70% identical to the nucleic acid sequence set forth in SEQ ID NO:14 (the nucleic acid sequence set forth in SEQ ID NO:14 encodes the protein set forth in SEQ ID NO:15); (5) a nucleic acid sequence that is at least 70% identical to the nucleic acid sequence set forth in SEQ ID NO:16 (the nucleic acid sequence set forth in SEQ ID NO:16 encodes the protein set forth in SEQ ID NO:17); and (6) a nucleic acid sequence that is at least 70% identical to the nucleic acid sequence set forth in SEQ ID NO:18 (the nucleic acid sequence set forth in SEQ ID NO:18 encodes the protein set forth in SEQ ID NO:19).

The term “regulatory element” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, enhancers, promoters, translation leader sequences, introns, and polyadenylation signal sequences.

The term “rhizosphere” refers to the environment adjacent to the roots of a plant, regardless of the identity of the substrate (such as soil or water) in which the plant is growing.

The term “functional fragment” when used in reference to an isolated protein of the invention refers to a fragment that is a portion of the full-length protein, provided that the fragment has a biological activity that is characteristic of the corresponding full-length protein.

The term “complement” when used in connection with a nucleic acid molecule refers to the complementary nucleic acid sequence as determined by Watson-Crick base pairing. For example, the complement of the nucleic acid sequence 5′CCATG3′ is 5′CATGG3′.

In one aspect, the present invention provides isolated nucleic acid molecules that encode one, or more, or all, of the enzymes (or a functional fragment thereof) required to produce PDTC. Thus, in one aspect, the present invention provides an isolated portion of the Pseudomonas stutzeri genome including the following open reading frames (ORFs): ORF-K (SEQ ID NO:1, encoding the protein set forth in SEQ ID NO:2); ORF-N (SEQ ID NO:3, encoding the protein set forth in SEQ ID NO:4); ORF-P (SEQ ID NO:5, encoding the protein set forth in SEQ ID NO:6); ORF-C (SEQ ID NO:7, encoding the protein set forth in SEQ ID NO:8); ORF-G (SEQ ID NO:9, encoding the protein set forth in SEQ ID NO:10); ORF-H (SEQ ID NO:11, encoding the protein set forth in SEQ ID NO:12); ORF-F (SEQ ID NO:14, encoding the protein set forth in SEQ ID NO:15); ORF-J (SEQ ID NO:16, encoding the protein set forth in SEQ ID NO:17); ORF-I (SEQ ID NO:18, encoding the protein set forth in SEQ ID NO:19); ORF-A (SEQ ID NO:20, encoding the protein set forth in SEQ ID NO:21); ORF-B (SEQ ID NO:22, encoding the protein set forth in SEQ ID NO:23); ORF-D (SEQ ID NO:24, encoding the protein set forth in SEQ ID NO:25); ORF-E (SEQ ID NO:26, encoding the protein set forth in SEQ ID NO:27); ORF-L (SEQ ID NO:28, encoding the protein set forth in SEQ ID NO:29); ORF-M (SEQ ID NO:30, encoding the protein set forth in SEQ ID NO:31); ORF-O (SEQ ID NO:32, encoding the protein set forth in SEQ ID NO:33); and ORF-Q (SEQ ID NO:34, encoding the protein set forth in SEQ ID NO:35).

In another aspect, the present invention provides isolated nucleic acid molecules that are at least 70% identical (such as at least 80% identical, or at least 90% identical, or at least 95% identical, or at least 99% identical) to a nucleic acid molecule selected from the group consisting of SEQ ID NO:1, SEQ ID NO:3, SEQ ID NO:5, and SEQ ID NO:7.

The present invention also provides isolated nucleic acid molecules that comprise a PDTC gene cluster as defined herein. The isolated nucleic acid molecules of the invention that comprise a PDTC gene cluster may optionally further comprise: a nucleic acid sequence that is at least 70% identical to the nucleic acid sequence set forth in SEQ ID NO:7; a nucleic acid sequence that is at least 70% identical (such as at least 80% identical, or at least 90% identical, or at least 95% identical, or at least 99% identical) to the nucleic acid sequence set forth in SEQ ID NO:9; and a nucleic acid sequence that is at least 70% identical (such as at least 80% identical, or at least 90% identical, or at least 95% identical, or at least 99% identical) to the nucleic acid sequence set forth in SEQ ID NO:11. The present invention further provides isolated nucleic acid molecules that are at least 70% identical (such as at least 80% identical, or at least 90% identical, or at least 95% identical, or at least 99% identical) to the nucleic acid molecule consisting of the nucleic acid sequence set forth in SEQ ID NO:13.

Nucleic acid molecules of the present invention can be isolated by a variety of cloning techniques known to those of ordinary skill in the art. For example, nucleic acid molecules having the nucleic acid sequences set forth herein (or portions thereof) can be used as hybridization probes utilizing, for example, the technique of hybridizing radiolabeled nucleic acid probes to nucleic acids immobilized on nitrocellulose filters or nylon membranes as set forth at pages 9.52 to 9.55 of Molecular Cloning, A Laboratory Manual (2^{nd} edition), Sambrook et al. eds., the cited pages of which are incorporated herein by reference. The hybridization probes may be labeled with appropriate reporter molecules. Exemplary means for producing specific hybridization probes include oligolabeling, nick translation, end-labelling or PCR amplification using a labeled nucleotide. Appropriate hybridization conditions can be readily calculated by one of ordinary skill in the art. For example, with respect to nucleic acid molecules greater than about 100 bases in length, typical stringent hybridization conditions are no more than 25° C. to 30° C. (for example, 10° C.) below the melting temperature (Tm) of the native duplex (see generally, Sambrook et al., Molecular Cloning: A Laboratory Manual, 2^{nd} Ed. Cold Spring Harbor Press, 1987; Ausubel et al., Current Protocols in Molecular Biology, Greene Publishing, 1987). Tm for nucleic acid molecules greater than about 100 bases can be calculated by the formula Tm=81.5+0.41% (G+C−log(Na⁺). With respect to nucleic acid molecules having a length less than 100 bases, exemplary stringent hybridization conditions are 5° to 10° C. below Tm. On average, the Tm of a nucleic acid molecule of length less than 100 bp is reduced by approximately (500/oligonucleotide length) degrees centigrade.

Oligonucleotides for hybridization screening may be designed based on the DNA sequences of one or more of the nucleic acid molecules of the invention disclosed herein. Oligonucleotides for screening are typically at least 11 bases long and more usually at least 20 or 25 bases long. In one embodiment, the oligonucleotide is 20-30 bases long. Such an oligonucleotide may be synthesized in an automated fashion. To facilitate detection, the oligonucleotide may be conveniently labeled, generally at the 5′ end, with a reporter molecule, such as a radionuclide, (e.g., ³²P), enzymatic label, protein label, fluorescent label, or biotin. A library is generally plated as colonies or phage, depending upon the vector, and the recombinant DNA is transferred to nylon or nitrocellulose membranes.

Hybridization conditions are tailored to the length and GC content of the oligonucleotide. Oligonucleotides for hybridization are typically at least 11 bases long, generally less than 100 bases long, and preferably at least 15 bases long, such as at least 20 bases long, or at least 25 bases long, and preferably 20-70, 25-50, or 30-40 bases long. Washing is initially performed at the same conditions as hybridization. If the background is unacceptably high, washing temperature is increased a few degrees until background is acceptable.

Following denaturation, neutralization, and fixation of the DNA to the membrane, membranes are hybridized with labeled probe. Suitable hybridization conditions may be found in Sambrook et al., supra, Ausubel et al., supra, and furthermore hybridization solutions may contain additives such as tetramethylammonium chloride or other chaotropic reagents or hybotropic reagents (e.g., ammonium trichloroacetate; see for example, WO 98/13527) to increase specificity of hybridization.

Following hybridization, suitable detection methods reveal hybridizing colonies or phage that are then isolated and propagated. Candidate clones or amplified fragments may be verified as containing a desired nucleic acid sequence by any of various means. For example, the candidate clones may be hybridized with a second, non-overlapping probe or subjected to DNA sequence analysis.

Again, by way of example, nucleic acid molecules of the present invention can be isolated by the polymerase chain reaction (PCR) described in The Polymerase Chain Reaction (Mullis et al., eds., Birkhauser Boston (1994)), incorporated herein by reference. Template genomic DNA can be obtained from any bacterial species, such as from any Pseudomonas species. Additionally, nucleic acid molecules of the present invention can be synthesized in an automated fashion.

Nucleotide sequence variants of nucleic acid molecules of the present invention are useful provided that they encode a protein that retains the biological activity of the wild-type protein. Nucleotide sequence variants are nucleic acid molecules with some differences in their sequences as compared to the corresponding, native, i.e., naturally-occurring, nucleic acid molecules. Ordinarily, the variants will possess at least about 70% identity with the corresponding native sequences, and preferably, they will be at least about 80% identical to the corresponding, native sequences. The nucleic acid sequence variants falling within this invention possess substitutions, deletions, and/or insertions at certain positions. Sequence variants may be used to attain desired enhanced or reduced activity, or altered temporal and spatial patterns of activity. Such sequence variants can be generated by a variety of art-recognized techniques.

By way of non-limiting example, the two primer system utilized in the Transformer Site-Directed Mutagenesis kit from Clontech (Palo Alto, Calif.), may be employed for introducing site-directed mutations into nucleic acid molecules of the present invention. Following denaturation of the target plasmid in this system, two primers are simultaneously annealed to the plasmid; one of these primers contains the desired site-directed mutation, the other contains a mutation at another point in the plasmid resulting in elimination of a restriction site. Second strand synthesis is then carried out, tightly linking these two mutations, and the resulting plasmids are transformed into a mutS strain of E. coli. Plasmid DNA is isolated from the transformed bacteria, restricted with the relevant restriction enzyme (thereby linearizing the unmutated plasmids), and then retransformed into E. coli. This system allows for generation of mutations directly in an expression plasmid, without the necessity of subcloning or generation of single-stranded phagemids. The tight linkage of the two mutations and the subsequent linearization of unmutated plasmids results in high mutation efficiency and allows minimal screening. Following synthesis of the initial restriction site primer, this method requires the use of only one new primer type per mutation site. Rather than prepare each positional mutant separately, a set of “designed degenerate” oligonucleotide primers can be synthesized in order to introduce all of the desired mutations at a given site simultaneously. Transformants can be screened by sequencing the plasmid DNA through the mutagenized region to identify and sort mutant clones. Each mutant DNA can then be fully sequenced or restricted and analyzed by electrophoresis on Mutation Detection Enhancement gel (J. T. Baker, Sanford, Me.) to confirm that no other alterations in the sequence have occurred (by band shift comparison to the unmutagenized control).

In other aspects, the present invention provides vectors that include one or more nucleic acid molecules of the invention, and host cells (including bacterial and plant cells) that include one or more vectors of the invention. Vectors useful for introducing the nucleic acid molecules of the invention into plant cells can be based, for example, on the Ti plasmid of Agrobacterium tumefaciens. The construction of suitable vectors containing nucleic acid molecules of the invention and optional elements, such as regulatory sequences and phenotypic selection genes utilize standard recombinant DNA procedures. Isolated plasmids and DNA fragments are cleaved, tailored, and ligated together in a specific order to generate the desired vectors, as is well known in the art (see, for example, Sambrook et al., Molecular Cloning: A Laboratory Manual, Cold Spring Harbor Laboratory Press (1989)). The nucleic acid molecules and vectors of the invention may be prepared by manipulating the various elements to place them in proper orientation. Thus, adapters or linkers may be employed to join the DNA fragments. Other manipulations may be performed to provide for convenient restriction sites, removal of restriction sites or superfluous DNA. These manipulations can be performed by art-recognized methods (see Sambrook et al., Molecular Cloning: A Laboratory Manual, Cold Spring Harbor Laboratory Press (1989)).

Prokaryotes may be used as host cells for routine genetic manipulation and/or construction of the nucleic acid molecules and vectors of the invention. They are particularly useful for rapid production of large amounts of DNA, for production of single-stranded DNA templates used for site-directed mutagenesis, for screening many mutants simultaneously, and for DNA sequencing of the mutants generated. Suitable prokaryotic host cells include E. coli K12 strain 94 (ATCC No. 31,446), E. coli strain W3110 (ATCC No. 27,325) E. coli X1776 (ATCC No. 31,537), and E. coli B; however many other strains of E. coli, such as HB101, JM101, NM522, NM538, NM539, and many other species and genera of prokaryotes including bacilli such as Bacillus subtilis, other enterobacteriaceae such as Salmonella typhimurium or Serratia marcesans, and various Pseudomonas species may all be used as hosts. Prokaryotic host cells or other host cells with rigid cell walls are generally transformed using the calcium chloride method as described in section 1.82 of Sambrook et al., supra. Alternatively, electroporation may be used for transformation of these cells. Prokaryote transformation techniques are set forth in Dower, in Genetic Engineering, Principles and Methods, 12:275-296, Plenum Publishing Corp. (1990); and Hanahan et al., Meth. Enzymol., 204:63 (1991).

In other aspects, the present invention provides cells (such as plant cells and bacterial cells) that include one or more vectors of the invention. Vectors of the invention can be introduced into plant cells using techniques well known to those skilled in the art. These methods include, but are not limited to, (1) direct DNA uptake, such as particle bombardment or electroporation (see, Klein et al., Nature 327:70-73 (1987); U.S. Pat. No. 4,945,050), and (2) Agrobacterium-mediated transformation (see, e.g., U.S. Pat. Ser. Nos: 6,051,757; 5,731,179; 4,693,976; 4,940,838; 5,464,763; and 5,149,645). Within the cell, the transgenic sequences may be incorporated within the chromosome. The skilled artisan will recognize that different independent insertion events may result in different levels and patterns of gene expression (Jones et al., EMBO J. 4:2411-2418 (1985); De Almeida et al., MGG 218:78-86 (1989)), and thus that multiple events may have to be screened in order to obtain lines displaying the desired expression level and pattern.

Transgenic plants can be obtained, for example, by transferring vectors that include a selectable marker gene, e.g., the kan gene encoding resistance to kanamycin, into Agrobacterium tumifaciens containing a helper Ti plasmid as described in Hoeckema et al., Nature, 303:179-181 (1983) and culturing the Agrobacterium cells with leaf slices, or other tissues or cells, of the plant to be transformed as described by An et al., Plant Physiology, 81:301-305 (1986). Transformation of cultured plant host cells is normally accomplished through Agrobacterium tumifaciens.

Transformed plant calli may be selected through the selectable marker by growing the cells on a medium containing, for example, kanamycin, and appropriate amounts of phytohormone such as naphthalene acetic acid and benzyladenine for callus and shoot induction. The plant cells may then be regenerated and the resulting plants transferred to soil using techniques well known to those skilled in the art.

In addition to the methods described above, several methods are known in the art for transferring cloned DNA into a wide variety of plant species, including gymnosperms, angiosperms, monocots and dicots (see, e.g., Glick and Thompson, eds., Methods in Plant Molecular Biology, CRC Press, Boca Raton, Fla. (1993), incorporated by reference herein). Representative examples include electroporation-facilitated DNA uptake by protoplasts in which an electrical pulse transiently permeabilizes cell membranes, permitting the uptake of a variety of biological molecules, including recombinant DNA (Rhodes et al., Science, 240:204-207 (1988)); treatment of protoplasts with polyethylene glycol (Lyznik et al., Plant Molecular Biology, 13:151-161 (1989)); and bombardment of cells with DNA-laden microprojectiles which are propelled by explosive force or compressed gas to penetrate the cell wall (Klein et al., Plant Physiol. 91:440-444 (1989) and Boynton et al., Science, 240(4858):1534-1538 (1988)). A method that has been applied to Rye plants (Secale cereale) is to directly inject plasmid DNA, including a selectable marker gene, into developing floral tillers (de la Pena et al., Nature 325:274-276 (1987)). Further, plant viruses can be used as vectors to transfer genes to plant cells. Examples of plant viruses that can be used as vectors to transform plants include the Cauliflower Mosaic Virus (Brisson et al., Nature 310:511-514 (1984); Other useful techniques include: site-specific recombination using the Cre-lox system (see, U.S. Pat. Ser. No. 5,635,381); and insertion into a target sequence by homologous recombination (see, U.S. Pat. Ser. No. 5,501,967). Additionally, plant transformation strategies and techniques are reviewed in Birch, R. G., Ann Rev Plant Phys Plant Mol Biol, 48:297 (1997); Forester et al., Exp. Agric., 33:15-33 (1997). The aforementioned publications disclosing plant transformation techniques are incorporated herein by reference, and minor variations make these technologies applicable to a broad range of plant species.

Positive selection markers may also be utilized to identify plant cells that include a vector of the invention. For example, U.S. Pat. Ser. Nos. 5,994,629, 5,767,378, and 5,599,670 describe the use of a beta-glucuronidase transgene and application of cytokinin-glucuronide for selection, and use of mannophosphatase or phosphmanno-isomerase transgene and application of mannose for selection.

The cells which have been transformed may be grown into plants by a variety of art-recognized means. See, for example, McConnick et al., Plant Cell Reports 5:81-84 (1986). These plants may then be grown, and either selfed or crossed with a different plant strain, and the resulting homozygotes or hybrids having the desired phenotypic characteristic identified. Two or more generations may be grown to ensure that the subject phenotypic characteristic is stably maintained and inherited and then seeds harvested to ensure the desired phenotype or other property has been achieved.

The following are representative plant species that are suitable for genetic manipulation in accordance with the present invention. The citations are to representative publications disclosing genetic transformation protocols that can be used to genetically transform the listed plant species. Rice (Alam, M. F. et al., Plant Cell Rep. 18:572-575 (1999)); maize (U.S. Pat. Ser. Nos. 5,177,010 and 5,981,840); wheat (Ortiz, J. P. A., et al., Plant Cell Rep. 15:877-881 (1996)); tomato (U.S. Pat. Ser. No. 5,159,135); potato (Kumar, A., et al., Plant J. 9:821-829 (1996)); cassava (Li, H-Q., et al., Nat. Biotechnology 14:736-740 (1996)); lettuce (Michelmore, R., et al., Plant Cell Rep 6:439-442 (1987)); tobacco (Horsch, R. B., et al., Science 227:1229-1231 (1985)); cotton (U.S. Pat. Ser. Nos. 5,846,797 and 5,004,863); grasses (U.S. Pat. Ser. Nos. 5,187,073 and 6,020,539); peppermint (X. Niu et al., Plant Cell Rep. 1765-171 (1998)); citrus plants (Pena, L. et al., Plant Sci. 104: 183-191 (1995)); caraway (F. A. Krens, et al., Plant Cell Rep., 17:39-43 (1997)); banana (U.S. Pat. Ser. No. 5,792,935; soybean (U.S. Pat. Ser. Nos. 5,416,011; 5,569,834; 5,824,877; 5,563,04455 and 5,968,830); pineapple (U.S. Pat. Ser. No. 5,952,543); poplar (U.S. Pat. Ser. No. 4,795,855); monocots in general (U.S. Pat. Ser. Nos. 5,591,616 and 6,037,522); brassica (U.S. Pat. Ser. Nos. 5,188,958; 5,463,174 and 5,750,871); and cereals (U.S. Pat. Ser. No. 6,074,877).

The vectors used in this inventions are introduced into plant cells by any suitable technique. If the marker gene is a selectable gene, only those cells that have incorporated the DNA package survive under selection with the appropriate phytotoxic agent. Progeny from the transformed plants may be tested to ensure that the DNA package has been successfully integrated into the plant genome. The presence of the stably integrated elements into the transformed parent plants may be ascertained, for example, by southern hybridization techniques or PCR analysis, known in the art.

The nucleic acid molecules and vectors of the invention can be introduced into any desired microbial species, such as, but not limited to: Bacillus, Deinococcus, Thermus, Caulobacter, Methylobacterium, Alcaligenes, Burkholderia, Thiobacillus, Shingomonas, Flavobacterium, Achromatium, Acinetobacter, Actinobacillus, Aeromonas, Azotobacteriaceae, Beggiotoaceae (Beggiatoa), Chromateaceae, Collwellia, Coxiella, Ectothiorhodspira, Enterobacteriaceae, Legionellaceae, Methylococcaeeae, Moraxellaceae, Pateurellaceae, Pseudomonas, Shewanella, Thiomicrospira, Thiothrix, Vibrionaceae and Micrococcus radioduranis. An exemplary method for introducing the nucleic acid molecules of the invention into a bacterium by conjugation is set forth in Example 2 herein.

In another aspect, the present invention provides isolated proteins encoded by the nucleic acid molecules of the invention. By way of representative example, the present invention provides isolated proteins that are at least 70% identical to one or more of the proteins consisting of the amino acid sequences set forth in SEQ ID NO:2, SEQ ID NO:4, SEQ ID NO:6, and SEQ ID NO:8. The proteins of the invention can be isolated, for example, by constructing a vector that includes a nucleic acid molecule that encodes the desired protein. The nucleic acid molecule is typically under the control of a regulatory element (e.g., an inducible promoter) that directs translation of the nucleic acid molecule. The vector is introduced into a cell (such as a bacterial cell, including bacteria of the genus Pseudomonas) and the expressed protein is purified therefrom. Representative examples of art-recognized techniques for purifying, or partially purifying expressed proteins of the invention from cells include ion-exchange chromatography, exclusion (gel permeation) chromatography, hydrophobic interaction chromatography, reversed-phase chromatography and immobilized metal affinity chromatography.

Hydrophobic interaction chromatography and reversed-phase chromatography are two separation methods based on the interactions between the hydrophobic moieties of a sample and an insoluble, immobilized hydrophobic group present on the chromatography matrix. In hydrophobic interaction chromatography the matrix is hydrophilic and is substituted with short-chain phenyl or alkyl nonpolar groups. The mobile phase is usually an aqueous salt solution. In reversed phase chromatography the matrix is silica that has been substituted with n-alkyl chains, usually C₄-C₁₈. The matrix is less polar than the mobile phase. The mobile phase is usually a mixture of water and a less polar organic modifier.

Separations on hydrophobic interaction chromatography matrices are usually done in aqueous salt solutions, which generally are nondenaturing conditions. Samples are loaded onto the matrix in a high-salt buffer and elution is by a descending salt gradient. Separations on reversed-phase media are usually done in mixtures of aqueous and organic solvents, which are often denaturing conditions. In the case of protein and/or peptide purification, hydrophobic interaction chromatography depends on surface hydrophobic groups and is carried out under conditions which maintain the integrity of the protein molecule. Reversed-phase chromatography depends on the native hydrophobicity of the protein and is carried out under conditions which expose nearly all hydrophobic groups to the matrix, i.e., denaturing conditions.

Ion-exchange chromatography is designed specifically for the separation of ionic or ionizable compounds. The stationary phase (column matrix material) carries ionizable functional groups, fixed by covalent bonding to the stationary phase. These fixed charges carry a counterion of opposite sign. This counterion is not fixed and can be displaced. Ion-exchange chromatography is named on the basis of the sign of the displaceable charges. Thus, in anion ion-exchange chromatography the fixed charges are positive and in cation ion-exchange chromatography the fixed charges are negative.

Retention of a molecule on an ion-exchange chromatography column involves an electrostatic interaction between the fixed charges and those of the molecule, binding involves replacement of the nonfixed ions by the molecule. Elution, in turn, involves displacement of the molecule from the fixed charges by a new counterion with a greater affinity for the fixed charges than the molecule, and which then becomes the new, nonfixed ion.

The ability of counterions (salts) to displace molecules bound to fixed charges is a function of the difference in affinities between the fixed charges and the nonfixed charges of both the molecule and the salt. Affinities in turn are affected by several variables, including the magnitude of the net charge (depends on pH) of the molecule and the concentration and type of salt used for displacement.

Solid-phase packings used in ion-exchange chromatography include cellulose, dextrans, agarose, and polystyrene. The exchange groups used include DEAE (diethylaminoethyl), a weak base, that will have a net positive charge when ionized and will therefore bind and exchange anions; and CM (carboxymethyl), a weak acid, with a negative charge when ionized that will bind and exchange cations. Another form of weak anion exchanger contains the PEI (polyethyleneimine) functional group. This material, most usually found on thin layer sheets, is useful for binding proteins at pH values above their pI. The polystyrene matrix can be obtained with quaternary amine functional groups for strong base anion exchange or with sulfonic acid functional groups for strong acid cation exchange. Intermediate and weak ion-exchange materials are also available. Ion-exchange chromatography need not be performed using a column, and can be performed as batch ion-exchange chromatography with the slurry of the stationary phase in a vessel such as a beaker.

Gel filtration is performed using porous beads as the chromatographic support. A column constructed from such beads will have two measurable liquid volumes, the external volume, consisting of the liquid between the beads, and the internal volume, consisting of the liquid within the pores of the beads. Large molecules will equilibrate only with the external volume while small molecules will equilibrate with both the external and internal volumes. A mixture of molecules (such as peptides) is applied in a discrete volume or zone at the top of a gel filtration column and allowed to flow through the column. The large molecules are excluded from the internal volume and therefore emerge first from the column while the smaller molecules, which can access the internal volume, emerge later. The volume of a conventional matrix used for protein purification is typically 30 to 100 times the volume of the sample to be fractionated. The absorbance of the column effluent can be continuously monitored at a desired wavelength using a flow monitor.

High Performance Liquid Chromatography (HPLC) is an advancement in both the operational theory and fabrication of traditional chromatographic systems. HPLC systems for the separation of biological macromolecules vary from the traditional column chromatographic systems in three ways; (1) the column packing materials are of much greater mechanical strength, (2) the particle size of the column packing materials has been decreased 5- to 10-fold to enhance efficiency of separation, and (3) the columns are operated at 10-60 times higher mobile-phase velocity. Thus, by way of non-limiting example, HPLC can utilize exclusion chromatography, ion-exchange chromatography, hydrophobic interaction chromatography, reversed-phase chromatography and immobilized metal affinity chromatography and analytical and preparative mode.

In other aspects, the present invention provides methods for reducing the amount of a metal in a substrate (such as soil or water). Representative examples of metals that can be removed in accordance with the methods of this aspect of the invention include: iron, copper, chromium, cobalt, nickel, zinc, cadmium, lead, plutonium, arsenic, gold, titanium, tin, palladium, neodymium, gallium, bismuth, scandium and manganese. One embodiment of the methods of the invention for reducing the amount of a metal in a substrate comprises the steps of: (a) introducing into a substrate, comprising a metal ion species, a plant comprising roots and a PDTC gene cluster, the plant possessing a mechanism for transporting the metal ion species into the roots; and (b) expressing the PDTC gene cluster in the plant roots to form PDTC under conditions that enable the plant to remove an amount of the metal ion species from the substrate that is greater than the amount of the metal ion species that the plant would remove in the absence of expression of the PDTC gene cluster in the plant roots.

Plants comprising a PDTC gene cluster can be generated by any art-recognized technique for stably introducing DNA molecules (such as a nucleic acid molecule including a PDTC gene cluster) into plant cells and regenerating plants from the modified plant cells. The nucleic acid molecule comprising the PDTC gene cluster is typically utilized as part of a vector that is capable of replicating and being stably maintained within plant cells. Representative vectors and examples of genetic manipulation techniques for introducing a PDTC gene cluster into plant cells are set forth supra in connection with the description of plant host cells that include a vector of the invention. Representative nucleic acid molecules of the invention that encode a PDTC gene cluster are also described herein.

The PDTC gene cluster is expressed in the roots of plants (and, optionally, may also be expressed in other plant organs and tissues) under the control of one or more regulatory elements active in at least some cells (preferably all cells) of the plant roots. Representative examples of regulatory elements useful in this aspect of the invention include the Cauliflower Mosaic Virus 35S promoter. PDTC at or near the surface of the plant roots binds metal ions present in the substrate (such as soil or water contaminated with metal ions) and thereby enhances the availability of the metal ions to the endogenous (or genetically engineered) metal ion transport system of the plant, thereby enabling the plant to remove an amount of one or more metal ion species from the substrate that is greater than the amount of the metal ion species that the plant would remove in the absence of expression of the PDTC gene cluster in the plant roots.

Another embodiment of the methods of the invention for reducing the amount of a metal in a substrate comprises the steps of: (a) introducing into the rhizosphere of a plant at least one bacterial species that comprises a PDTC gene cluster, the rhizosphere comprising a metal ion species and the plant possessing a mechanism that transports the metal ion from the rhizosphere into the plant roots; (b) culturing the plant and the at least one bacterial species in the substrate for a time and under conditions that enable the bacterial species to synthesize PDTC and thereby increase availability of the metal ion to the plant roots so that the plant removes an amount of the metal ion from the substrate that is greater than the amount of the metal ion that the plant would remove if the bacterial species expressing PDTC was not present in the rhizosphere.

The at least one bacterial microorganism that comprises a PDTC gene cluster can be constructed by introducing into a desired bacterial species one or more nucleic acid molecules that include a PDTC gene cluster. The nucleic acid molecule(s) comprising the PDTC gene cluster is typically incorporated into a vector that is capable of replicating and being stably maintained within the target microorganism(s). Vectors useful in this aspect of the invention typically include (a) a nucleic acid molecule comprising the PDTC gene cluster, (b) a selectable marker gene (typically an antibiotic-resistance gene, such as a gene that confers resistance to ampicillin or kanamycin) for identification and recovery of microorganisms that incorporate the PDTC gene cluster, and (c) appropriate regulatory elements to control expression of the PDTC biosynthetic genes and selectable marker gene(s). Representative examples of inducible promoters useful for regulating the expression of the PDTC biosynthetic genes and/or the selectable marker gene(s) are Ptac (the activity of which is induced by IPTG) and Pm (the activity of which is induced by m-toluic acid). (See, e.g., Marques et al., Mol. Microbiol. 9(5):923-929 (1993); Yap et al., J. Bacteriol, 176(9):2603-2610 (1994); and Cebolla et al., Appl. Environ. Microbiol., 61(2):660-668 (1995)).

The nucleic acid molecules of the invention can be introduced into any desired microbial species, such as, but not limited to: Bacillus, Deinococcus, Termus, Caulobacter, Methylobacterium, Alcaligenes, Burkholderia, Thiobacillus, Shingomonas, Flavobacterium, Achromatium, Acinetobacter, Actinobacillus, Aeromonas, Azotobacteriaceae, Beggiotoaceae (Beggiatoa), Chromateaceae, Collwellia, Coxiella, Ectothiorhodspira, Enterobacteriaceae, Legionellaceae, Methzylococcaeeae, Moraxellaceae, Pateurellaceae, Pseudomonas, Shewanella, Thiomicrospira, Thiothrix, Vibrionaceae and Micrococcus radiodurans. An exemplary method for introducing the nucleic acid molecules of the invention into a bacterium is set forth in Example 2 herein.

PDTC (synthesized by the bacteria that have been engineered to include a PDTC gene cluster) at or near the surface of the plant roots binds metal ions present in the substrate (such as soil or water contaminated with metal ions) and thereby enhances the availability of the metal ions to the plant's metal ion transport system, enabling the plant to remove an amount of one or more metal ion species from the substrate that is greater than the amount of the metal ion species that the plant would remove if the bacterial microorganism expressing PDTC was not present.

In a related aspect, the present invention provides methods for immobilizing metal ions in a substrate. In this aspect of the invention, one or more species of microorganism, comprising a PDTC gene cluster, are introduced into a substrate containing one or more metal ions under conditions that enable expression of the PDTC gene cluster to form PDTC which is released into the substrate. The PDTC binds the metal ions thereby reducing the extent of movement (such as by diffusion) of the metal ions through the substrate. Methods of this aspect of the invention are useful, for example, for treating soil contaminated with metal ions, thereby reducing the ability of the metal ions to move throughout the soil.

The complex formed between PDTC and Cu(II) ions is capable of degrading carbon tetrachloride to yield carbon dioxide and hydrogen chloride. Thus, the present invention provides methods for degrading carbon tetrachloride, such as carbon tetrachloride contaminating soil or water.

One embodiment of the methods of the invention for degrading carbon tetrachloride includes the steps of: (a) introducing into a substrate a plant comprising roots and a PDTC gene cluster, the substrate further comprising Cu(II) ions and carbon tetrachloride that are introduced into the substrate, before, after or simultaneously with the introduction of the plant into the substrate; and (b) expressing the PDTC gene cluster in the plant roots under conditions that enable the plant to synthesize PDTC and release the PDTC into the substrate, thereby chemically degrading the carbon tetrachloride by the action of a complex formed between the PDTC and the Cu(II) ions.

Another embodiment of the methods of the invention for degrading carbon tetrachloride includes the steps of: (a) introducing into a substrate, comprising Cu(II) ions and carbon tetrachloride, at least one bacterial species that comprises a PDTC gene cluster under conditions that enable expression of the PDTC gene cluster to form PDTC; and (b) release of the PDTC into the substrate thereby chemically degrading the carbon tetrachloride by the action of a complex formed between the PDTC and the Cu(II) ions. The Cu(II) ions are introduced into the substrate before, after, or simultaneously with the bacterial species.

Another embodiment of the methods of the invention for degrading carbon tetrachloride in a substrate includes the steps of: (a) introducing into a substrate, comprising carbon tetrachloride and Cu(II) ions, a nucleic acid molecule comprising a PDTC gene cluster comprising a plurality of PDTC biosynthetic genes, each of the PDTC biosynthetic genes being operably linked to at least one regulatory element that directs their expression within a microorganism; (b) uptake of the introduced nucleic acid molecule by a microorganism; (c) expression of the PDTC gene cluster within the microorganism to form PDTC; and (d) release of the PDTC into the substrate thereby chemically degrading the carbon tetrachloride by the action of a complex formed between the PDTC and the Cu(II) ions. In this embodiment of the methods of the invention, the nucleic acid molecule comprising the PDTC gene cluster can be introduced into soil by attachment (such as by absorption or adsorption) of the nucleic acid molecule to clay particles. Clay useful in this aspect of the invention includes colloidal clays such as bentonite and montmorillonite. Colloidal sand particles (such as colloidal sand produced by grinding pure silica) is also useful in this aspect of the invention. Typically, the particle size of the clay or sand is less than 0.002 mm.

The nucleic acid molecule comprising the PDTC gene cluster can be attached to the clay or sand particles by any art-recognized method, such as the methods described in Lorenz, M. G., et al., J. Gen. Microbiol. 134:107-112 (1988); Sikorski, J., et al., Microbiology 144:569-576 (1998); Lorenz, M. G., and Wackernagel, Arch. Microbiol. 154:380-385 (1990); Khanna, M., and Stotzky, G, Appl. Environ. Microbiol. 58:1930-1939 (1992); Nielsen, K. M., et al., Appl. Environ. Microbiol. 63:1945-1952 (1997); Romanowski, G., et al., Mol. Ecol. 2:171-181 (1993); Romanowski, G., et al., Appl. Environ. Microbiol. 57:1057-1061 (1991). Once introduced into the soil, the nucleic acid molecule is taken up by one or more species of microorganism (typically bacteria) by natural processes.

The nucleic acid molecule comprising the PDTC gene cluster is typically utilized as part of a vector that is capable of replicating and being stably maintained within the target microorganism(s). Vectors useful in this aspect of the invention typically include (a) a nucleic acid molecule comprising the PDTC gene cluster, (b) a selectable marker gene (typically an antibiotic-resistance gene, such as a gene that confers resistance to ampicillin or kanamycin) for identification and recovery of microorganisms that incorporate the PDTC gene cluster, and (c) appropriate regulatory elements to control expression of the PDTC biosynthetic genes and selectable marker gene(s). Representative examples of inducible promoters useful for regulating the expression of the PDTC biosynthetic genes and/or the selectable marker gene(s) are Ptac (the activity of which is induced by IPTG) and Pm (the activity of which is induced by m-toluic acid).

In another aspect, the present invention provides compositions (such as the DNA molecule consisting of the sequence set forth in SEQ ID NO:13) and methods for transferring genes between bacterial species. Thus, for example, the DNA molecule consisting of the sequence set forth in SEQ ID NO:13 can be used to transfer genes included therein between species of Pseudomonas.

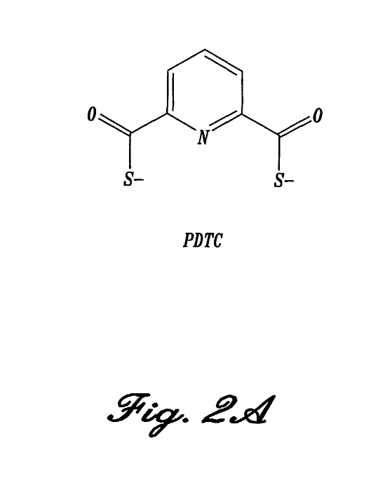

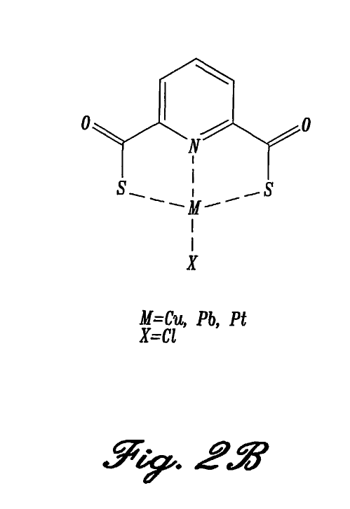

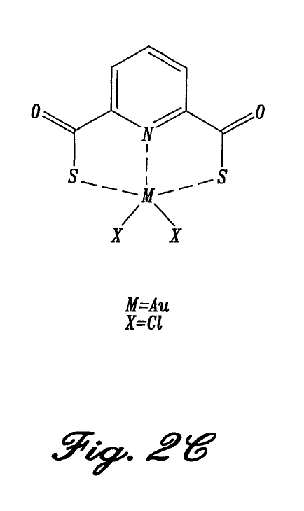

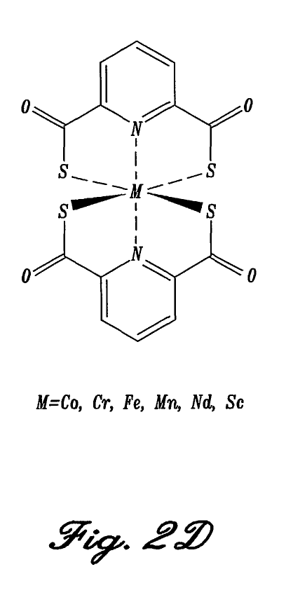

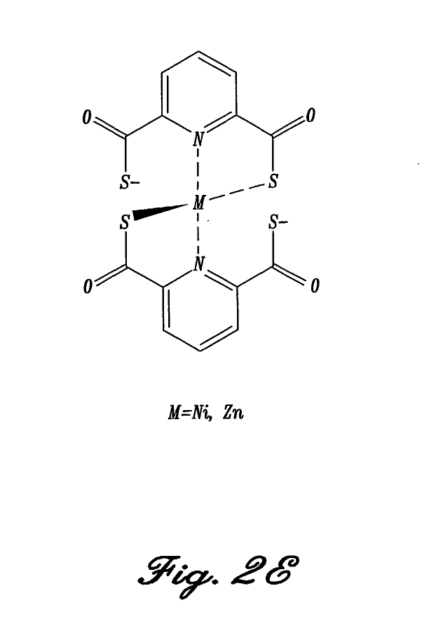

In other aspects of the invention, PDTC and certain of its metal complexes can be used to degrade carbon tetrachloride and for removing metals from substrates, such as soil and water. These advantageous properties render PDTC and certain of its metal complexes useful in environmental remediation methods including, for example, phytoremediation, bioaccumulation, water purification, waste water purification, solution mining mobilization, immobilization, detoxification, redox state modifier, and modification of metal ion reactivity. The chemical structure of PDTC is illustrated in FIG. 2A. Modes of PDTC binding to certain metals are illustrated in FIGS. 2B-2E, which show chemical structures of representative PDTC metal complexes.

As noted above, the present invention provides compositions and methods for degrading carbon tetrachloride. The compositions and methods of the invention useful in carbon tetrachloride degradation include PDTC and certain of its metal complexes. Representative PDTC complexes that are useful in carbon tetrachloride degradation include the PDTC copper (II) complex and PDTC cobalt complex.

In general, PDTC copper (II) complex can be a carbon tetrachloride contaminated substrate, such as soil or water, to facilitate carbon tetrachloride degradation. The amount of PDTC copper complex applied to the substrate will depend on the substrate and the amount of carbon tetrachloride present in the substrate. The PDTC copper complex can be applied as a solution (such as a saturated solution in water) or as a solid. The PDTC copper complex can also be used, for example, in an organic solvent such as methanol or dimethylformamide.

The complex formed between PDTC and Cu(II) ions is capable of degrading carbon tetrachloride to yield carbon dioxide and hydrogen chloride. Thus, the present invention provides methods for degrading carbon tetrachloride, such as carbon tetrachloride contaminating soil or water.

In one embodiment, the method for degrading carbon tetrachloride includes the steps of: (a) contacting PDTC copper (II) complex with a substrate contaminated with carbon tetrachloride; and (b) chemically degrading the carbon tetrachloride by the action of a complex.

Carbon tetrachloride degradation by PDTC is described in Example 3. As described in Example 3, carbon tetrachloride dechlorination by PDTC requires copper and is inhibited by cobalt, but not by iron or nickel. PDTC reacts stoichiometrically, rather than catalytically, without added reducing equivalents. With added reductants, an increased turnover was seen, along with increased chloroform production.

Carbon tetrachloride (CCl₄) and its dechlorination products have been thoroughly studied, primarily to understand their toxic and carcinogenic effects in mammals, and their environmental fate. In the liver, CCl₄ is transformed by cytochrome P₄₅₀ through reductive mechanisms. Reactive species such as trichloromethyl radical and dichlorocarbene, thought to be responsible for cell damage due to CCl₄ exposure, are produced. Microbial transformations of CCl₄ have also been studied to evaluate the potential for engineered bioremediation or natural attenuation of CCl₄-contaminated environments. The only biochemical agents from microbial sources that have been studied for their CCl₄ dechlorination activity are tetrapyrrole-type cofactors, including cobalamius, porphyrins, and factor F₄₃₀. These cofactors also mediate the transformation of CCl₄ through a reductive mechanism.

The data on dehalogenation mechanisms involving transition metal cofactors, which include product analyses and spectroscopic studies, indicate an initial one-electron reduction to give radical species. The dominant fate of the resulting carbon-centered radicals in these systems is another one-electron reduction by the bulk reductant, which is present to regenerate the active form of the cofactor. This net two-electron reduction yields hydrogenolytic products (i.e., replacement of one chlorine atom by one hydrogen atom). Other products resulting from the net two-electron reduction of CCl₄ are carbon monoxide and formate, which arise through hydrolysis of dichlorocarbene.

Another distinct type of CCl₄ dechlorination activity has been described in cultures of iron-limited Pseudomonas stutzeri strain KC. This activity is characterized by an extensive hydrolysis that gives CO₂ as a major product, as well as uncharacterized non-volatile material and low or undetectable levels of chloroform. Chloroform is not dechlorinated by this organism, indicating that CCl₄ is degraded via a novel pathway that avoids the accumulation of less-chlorinated products. Thiophosgene (CSCl₂) was identified as an intermediate of the net hydrolysis in our earlier studies. Quantitative data obtained using trapping agents demonstrated that the pathway involving thiophosgene accounts for most of the CCl₄ transformation observed in strain KC cultures under anoxic conditions. Oxygen substitution at the carbon atom of CCl₄ was also observed in the form of carbonyl-containing products, which were found to increase when O₂ was present. If trichloromethyl radical were involved, oxygen substitution could be attributed to a phosgene intermediate, likely to occur in the presence of O₂, but not under anoxic conditions. Another intermediate explaining carbonyl substitution under anoxic conditions and arising from thiophosgene hydrolysis is carbonyl sulfide (COS), which is also likely to be trapped by the nucleophiles used. An abiotic CCl₄ transformation which affects the substitution of sulfur for chlorine occurs in mineral/sulfide mixtures. The data in those studies suggested a radical substitution mechanism, initiated by one-electron reduction of CCl₄ at a metal center and followed by reaction of trichloromethyl radical with one of a variety of sulfur species that may have been present.

Pyridine-2,6-bis(thiocarboxylic acid) is an extracellular agent responsible for CCl₄ dechlorination activity in strain KC. PDTC has been identified as a metal-chelating agent from iron-limited cultures of a strain of Pseudomonas putida. The occurrence of two thiocarboxylic groups in PDTC and its ability to coordinate transition metals suggested a potential mechanism for reaction with CCl₄ analogous to that proposed for the mineral/sulfide system; specifically, reduction at the metal center to produce trichloromethyl radical, and condensation of this radical with one of the sulfur atoms of PDTC. The addition of certain transition metals to cultures of P. stutzeri strain KC has been found to exert profound effects on CCl₄ transformation activity. Fe(II) and Fe(III) prevented CCl₄ transformation when present initially in culture media at 10-100 μM, but not when added to cultures already showing this activity. Co(II) was found to inhibit CCl₄ transformation in low micromolar concentrations, and to inhibit growth at higher concentrations. CuCl₂ stimulated CCl₄ transformation activity at very low concentrations (5 nM), and had an inhibitory effect on growth of bacteria at higher concentrations. Inhibition of dechlorination may be due to the formation of inactive metal-containing complexes in preference to the active dechlorinating species. This scenario did not explain the data obtained from iron supplementation experiments. An alternative hypothesis was that inhibition or stimulation of dechlorination activity might be due to repression or induction of PDTC biosynthesis, respectively. Data obtained from transposon mutants derived from strain KC indicate a repression of genes necessary for CCl₄ transformation in response to iron supplementation. The hypotheses for direct chemical effects of transition metals and physiological effects are not mutually exclusive; however, previous experiments have not allowed the clear resolution of these effects.

Another unexplained phenomenon was observed in studies attempting to characterize the extracellular dechlorination agent. These studies showed that either bacterial cells or a chemical reductant were required in order to observe dechlorination in culture supernatants. The rationale for the use of a chemical reductant was that the responsible agent might be a redox catalyst that could couple oxidation of a chemical reductant or cell-derived reducing equivalent to reductive dechlorination of CCl₄. This rationale was weakened upon determining that the agent itself contained sulfur and was the likely source of the sulfur atom transferred to the CCl₄ carbon atom, thus indicating a stoichiometric rather than catalytic reaction with respect to PDTC. The requirement for reductant then became somewhat puzzling. Questions regarding the effects of transition metals on PDTC dechlorination activity, and its chemical requirements, have important ramifications for the in situ use of PDTC in biological or chemical reactive treatments. The identification of PDTC as the active agent and the availability of chemically-synthesized PDTC has made experiments possible that can resolve chemical from biological effects and allow product analyses without the complications arising from the presence of bacterial cells.

Contamination of soils and water with metals is a problem in many areas of the world. Applying existing cleanup methods to these sites is expensive. Phytoextraction is a remediation strategy to remove metals from the soil by virtue of metal uptake into plant tissues and subsequent removal of that plant material. Development of a system employing plants to remove metals from the soil could substantially reduce the cost of remediating metal-contaminated sites. Augmenting current and future phytoremediation systems with microbial partners possessing exploitable traits such as excretion of specific metal-chelating molecules may be a way to speed up and broaden the application of phytoextraction techniques to existing contamination problems.

Certain cultivars of Indian mustard (Brassica juncea) have been found to accumulate significant levels of Cr, Cd, Ni, Zn, Cu, and Pb (58-, 52-, 31-, 17-, 7-, and 1.7-fold, respectively) in harvestable tissues when grown in metal-amended soil, as measured by phytoextraction coefficient (PC). The phytoextraction coefficient for a specific metal is determined by dividing the μg of metal per gram of dried harvestable plant tissue by the μg metal per gram of dry soil ((μg of metal/g DW plant tissue)/(μg of metal/g DW soil)).

Amending soils with metal-chelating molecules has been shown to be effective in substantially increasing Pb uptake and its subsequent translocation to shoot tissues of corn, pea, ragweed, goldenrod, and sunflower. Increased uptake of Cd, Zn, Mn, Cu, Fe, Al, and Ni has also been observed when bushbeans were grown in the presence of synthetic chelators. Metal-chelating molecules are thought to act by increasing the solubility of metals and thereby increasing the bioavailability of the metals for uptake and translocation by plants. Se and Hg uptake into the tissues of saltmarsh bulrush and rabbitfoot grass has been shown to be higher when naturally occurring rhizosphere bacteria are present than under axenic conditions. This mode of action of this enhancement was not elucidated by the researchers, but may be due to siderophore production by rhizosphere bacteria. Siderophores are iron-chelating molecules secreted by bacteria.

PDTC is a small molecule secreted by certain pseudomonads when subjected to iron-limiting conditions which forms complexes with many metals. PDTC is a candidate for phytoextraction enhancement studies for several reasons. Most siderophores chelate iron(III) with hydroxymate or catecholate ligands, but PDTC binds metals with various combinations of its pair of (thiocarboxylate) ligands combined with a single secondary amine (see FIGS. 2A-2E). Iron is chelated by most siderophores using 6 coordinating ligands. However, while most siderophores utilize 3 bi-dentate ligands, PDTC uses 2 tri-dentate ligands. At 197 daltons, PDTC is smaller than most bacterially produced siderophores, which have molecular weights in the range of 500 to 1000 daltons. Since PDTC is a small and relatively simple molecule it can be produced economically, either by bacterial cultures or synthetically. Its small size also increases the diffusion rate of the free molecule and its chelates. PDTC has an affinity to a wide range of metals including Au, Cd, Co, Cr, Cu, Fe, Mn, Nd, Ni, Pb, Pd, Sc, and Zn. As noted above, PDTC can also be produced in situ by several pseudomonads possibly including those which preferentially inhabit the rhizosphere of any given plant species. Because of its unique properties, PDTC can be used to expand the spectrum of metal contamination problems that can be addressed by phytoextraction.

Because PDTC is a metal chelator and forms strong metal complexes, PDTC can be used to bind and remove metal ions from a substrate. Thus, in another aspect of the invention, compositions and methods for chelating metals ions are described. These compositions and methods include PDTC and can be used in environmental remediation programs including, for example, phytoremediation, bioaccumulation, water purification, and solution mining, among others. In these methods, PDTC-containing compositions can be directly applied to metal-containing substrates, such as soil or water systems.

The usefulness of chelators in bioremediation efforts often depends on their ability to bind hazardous metal ions. Since most known microbial chelators have a high specificity toward iron, their value as bioremediation agents is severely diminished due to the relatively high availability of iron compounds in many natural settings, allowing iron to outcompete other metals for binding to the chelator. Advantageously, PDTC complexes are formed with numerous transition and heavy metals, lanthanides and actinides. PDTC is soluble in water or supercritical fluids, and can function in these solvents as an extractant of metals from most or all biological tissues and many environmental matrixes including, for example, groundwater, soils, and ores. PDTC complexes can be formed with transition metals, lanthanide metals, actinide metals, heavy metals, and radionuclides. Representative metals that could be complexed with PDTC include Cu, Sc, Ti, Mn, Ni, Cu, Fe, Zn, Cr, Co, As, Au, Pd, Cd, Pb, Hg, Nd, Tc, Sr, tin, gallium and bismuth, among others.

The present invention provides methods for immobilizing metal ions in a substrate. In this aspect of the invention, PDTC is contacted with a substrate containing one or more metal ions. The PDTC binds the metal ions thereby reducing the extent of movement (such as by diffusion) of the metal ions through the substrate. Methods of this aspect of the invention are useful, for example, for treating soil contaminated with metal ions, thereby reducing the ability of the metal ions to move throughout the soil. In one embodiment, the method is a phytoremediation method. In such a method, a plant having an ability to take up the PDTC metal complex can be introduced into the substrate (i.e., soil) such that the metal ion, through its complexation with PDTC, ultimately resides in the plant. The plant containing the metal ion can then be removed from the substrate thereby remediating the substrate.

The stability of various PDTC-metal complexes was determined by experiments on potentiometric and spectrophotometric studies of PDTC with several metals. The stability constants and relative binding strengths for PDTC and several of the physiologically important metals that it binds are described in Example 4.

To summarize, PDTC metal chelating was studied by potentiometric and spectrophotometric techniques as described in Example 4. The first two stepwise stability constants (log K) for successive proton addition to PDTC were found to be 5.48 and 2.58. The third stepwise stability protonation constant was estimated to be 1.3. The stability constant for cobalt(III), copper(II), nickel(II), and iron(III) were determined spectrophotometrically by competition experiments. The stability constants (log K) are, respectively, 33.93 (Co), 33.28 (Ni), and 33.36 (Fe). The constant for ferric PDTC was determined by competitive experiments with the hydroxide ion. Constants for cobalt(III), copper(II), and nickel(II) were determined by metal-metal competition with respect to the stability constant of ferric PDTC.

The following examples merely illustrate the best mode now contemplated for practicing the invention, but should not be construed to limit the invention.

### EXAMPLES

### Example 1

This example describes the cloning and characterization of a fragment (SEQ ID NO:13) of the Pseudomonas stutzeri genome that encodes enzymes involved in the biosynthesis of PDTC.

Characterization of strain CTN1, a spontaneous mutant defective in CCl₄ transformation: While studying CCl₄ transformation by P. stutzeri strain KC, the inventors noted a loss of this activity in a working stock of this organism. The loss of activity could not be attributed to medium composition; in fact, when frozen stocks of strain KC were revived and tested on the same medium (DRM), the normal activity was seen (not shown). It was subsequently determined that no detectable PDTC was produced by the inactive culture (CTN1; Table 1).

TABLE 1
PDTC production by strain KC and complementations
of strain

| | | [PDTC] |
|---|---|---|
| Host strain | Plasmid | (μmoles/mg protein) |
| P. stutzeri strain KC | none | 28.4 ± 4.6 |
| P. stutzeri CTN1 | none | N.D. |
| P. stutzeri CTN1 | pM22 | 2.55 ± 0.33 |
| P. stutzeri CTN1 | pM36 | N.D. |
| P. stutzeri CTN1 | pO9 | N.D. |
| P. stutzeri CTN1 | pT31 | 49.8 ± 2.9 |
| P. stutzeri CTN1 | pJS18 | 53.0 ± 1.1 |
| P. stutzeri CTN1 | pJS20 | 45.1 ± 11.3 |
| P. stutzeri CTN1 | pJS22 | 54.1 ± 19.8 |
| P. stutzeri CTN1 | pJS27 | 57.7 ± 3.3 |
| P. stutzeri CTN1 | pJS29 | 53.2 ± 7.6 |
| P. stutzeri CTN1 | pJS34 | N.D. |
| P. stutzeri CTN1 | pJS40 | 3.5 ± 0.5 |
| P. stutzeri CTN1 | pJS42 | 56.1 ± 7.5 |
| P. stutzeri CTN1 | pJS43 | 40.3 ± 0.6 |
| P. stutzeri CTN1 | pJS52 | 55.6 ± 3.3 |
| P. stutzeri CTN1 | pJS55 | 35.1 ± 0.4 |
| P. stutzeri CTN1 | pJS59 | N.D. |
| P. stutzeri CTN1 | pJS60 | N.D. |
| P. stutzeri CTN1 | pJS63 | 55.9 ± 16.9 |
| P. stutzeri CTN1 | pJS68 | N.D. |
| P. stutzeri CTN1 | pT-phoA1 | N.D. |

CTN1 by pT31 (SEQ ID NO: 13) and derivatives of pT31.
N.D. = not detected

The inventors have since observed this loss of CCl₄ transformation activity on several occasions (not shown). Further correlation of PDTC biosynthesis and CCl₄ dehalogenation was demonstrated by P. putida DSM3601, the culture from which PDTC was originally identified (Ockels, W., Römer, A., and Budzikiewicz, H., Tetrahedron Lett. 3341-3342 (1978)), which shows CCl₄ transformation activity in an iron-limited medium (not shown). The loss of PDTC production (Pdt⁻phenotype) during maintenance of strain KC led the inventors to suspect a defect in a genetic locus required for PDTC biosynthesis. However, the rapid CCl₄ transformation activity of strain KC (≧1 μg CCl₄ ml⁻¹ day⁻¹; Lewis, T. A., and Crawford, R. L., “Physiological factors affecting carbon tetrachloride dehalogenation by the denitrifying bacterium Pseudomonas sp. strain KC” Appl. Environ. Microbiol. 59:1635-1641 (1993)) was the only known phenotype distinguishing strain KC from other strains of P. stutzeri and therefore, the inventors could not easily rule out contamination. Consequently, to ensure that the inactive culture was that of a clonal variant of strain KC and not a random contaminant, several tests were performed. Biolog™ substrate utilization plates reproducibly identified the culture as P. stutzeri (data not shown). A partial sequence of the 16S ribosomal RNA subunit genes of strain KC (GenBank accession No. AF063219) and the Pdt⁻variant showed no differences in an approximately 500 base-pair segment. Genomic fingerprinting with rare-cutting restriction enzymes and pulsed-field agarose gel electrophoresis (PFGE) has been shown to readily resolve strains of P. stutzeri (Ginard et al. “Genome organization of Pseudomonas stutzeri and resulting taxonomic and evolutionary considerations” Int. J. Syst. Bact. 47:132-143 (1997)). Comparing strain KC and the Pdt⁻ variant by this technique clearly showed their clonal relatedness. The Pdt⁻ variant was then designated as strain CTN1.

Identification of a chromosomal deletion in strain CTN1: The appearance of a variant in PDTC biosynthesis without intentional selection suggested that a mutation characterized by a relatively high frequency had occurred. Such mutations could include plasmid loss, transposon or phage excision, as well as homologous (legitimate) recombination leading to deletion or inversion of chromosomal DNA. PFGE of undigested DNA did not indicate the presence of plasmids in strain KC or CTN1 (not shown), though this type of analysis did not rule out the existence of very large plasmids that did not enter the gel. The SpeI restriction patterns of the two strains differed discernibly only with respect to one band, of approximately 148 kb. This difference could be explained by a number of possible scenarios including point mutation, inversion, or deletion. To see if a gross rearrangement of the chromosome had occurred, we used extremely rare-cutting restriction enzymes. The intron-encoded I-CeuI, whose recognition site is found in bacterial rrl genes, gives four large fragments of P. stutzeri chromosomes, designated in descending size as CeA through CeD (Ginard et al. “Genome organization of Pseudomonas stutzeri and resulting taxonomic and evolutionary considerations” Int. J. Syst. Bact. 47:132-143 (1997)). Analyses of several pulsed-field gels revealed a dimorphism in the CeB fragment corresponding to a loss of 172±54 kb in CTN1. Dimorphism of the third largest PacI fragment (PaC) and loss of the PaE and PaF fragments corresponded to a net loss of 171±11 kb in strain CTN1. To locate the deletion on a circular map of the strain KC chromosome, Southern blotting analysis was performed. PFGE separations of single, as well as double, I-CeuI and PacI digests of chromosomal DNAs of strains KC and CTN1 were probed with a set of cloned loci, or PFGE-purified PaC, PaD, PaE, PaF fragments. This allowed construction of a crude map and localization of the deletion.

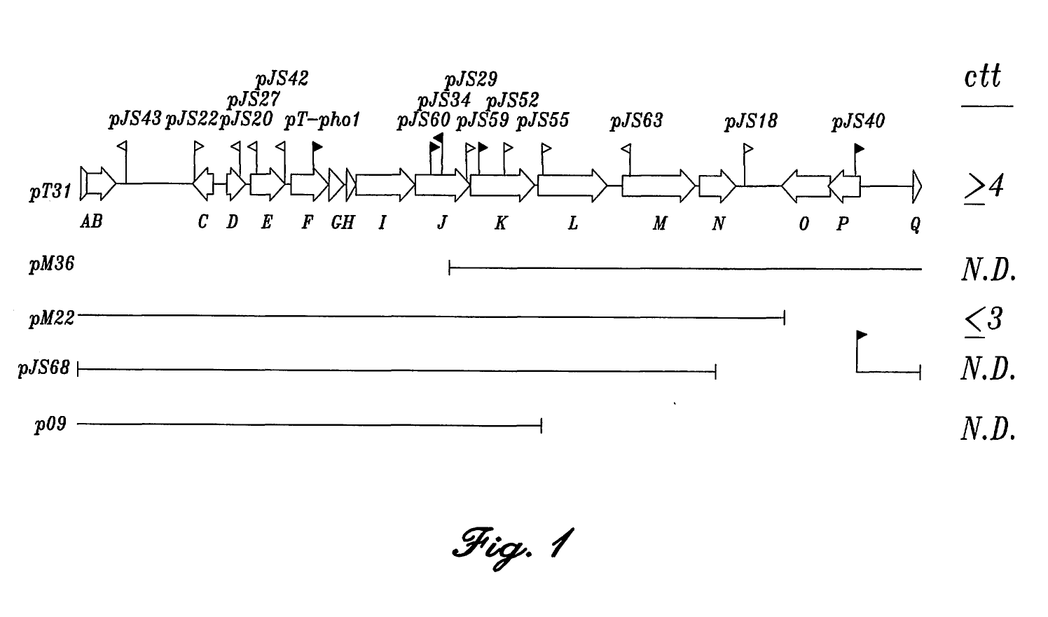

Cloning of pdt genes of strain KC: Chromosomal rearrangements may be a common event in pseudomonads and it was possible that the deletion present in CTN1 was unrelated to the Pdt⁻ phenotype. A more detailed study of the DNA deleted in strain CTN1 was undertaken to determine if it was involved in determining the Pdt phenotype. To obtain cloned DNA from the deleted region we used the 148 kb Spe1 fragment of strain KC as a probe to screen a strain KC genomic library. Several cosmids were identified and transferred into CTN1 by conjugation. The transconjugants were then tested for complementation of the Pdt⁻ phenotype by assaying CCl₄ transformation. One cosmid, pT31, was found to complement the Pdt⁻ phenotype, restoring PDTC production (Table 2) and CCl₄ transformation activity to CTN1. A map of the pT31 insert (SEQ ID NO:13), showing the locations of open reading frames, is set forth in FIG. 1.

TABLE 2
PDTC production by non-PDTC-producing heterologous
hosts of P. stutzeri strain KC pdt genes.

| | [PDTC]in medium (μmoles/mg protein) | |
|---|---|---|
| Host organism | Cosmid pRK311 | Cosmid pT31 |
| P. stutzeri CTN1 | N.D. | 49.8 ± 2.9 |
| P. putida mt-2 | N.D. | 100.5 ± 11.1 |
| P. fluorescens F113 | N.D. | 71.5 ± 30.9 |
| P. aeruginosa PAO1 | N.D. | 34.8 ± 8.8 |
| P. stutzeri ATCC 17588 | N.D. | 5.3 ± 2.7 |
| R. meliloti | N.D. | N.D. |
| E. coli ATCC 25922 | N.D. | N.D. |

PRK311 = cosmid vector with no insert.
N.D. = not detected.

This complemented mutant probably produced higher amounts of PDCT and greater CCl₄ transformation activity than strain KC because of the increased copy number of the cosmid-borne insert DNA. pT31 was found to contain an insert of 25,746 bp of DNA (SEQ ID NO:13) that was colinear with the strain KC chromosome. pT31 was used in cosmid walking experiments to align other clones tested for complementation and to localize the necessary region (FIG. 1). pM22, another cosmid with an approximately 25 kb genomic DNA insert, restored only 10% of wild-type level of PDTC production and a reduced level of CCl₄ transformation activity (FIG. 1, Table 1). Southern blotting with the entire pT31 molecule as a probe showed that sequences included on this construct were not present in CTN1, indicating that it lay entirely within the deletion. pT31 also did not hybridize with genomic DNAs from any of three culture collection strains of P. stutzeri (ATCC 11607, ATCC 14405, and ATCC 17588) (not shown). As this region was shown by deletion and complementation to be required for PDTC biosynthesis, it was designated the pdt locus.

The ability of pT31 to confer PDTC production and rapid CCl₄ transformation activity was not limited to strain CTN1 as host, but was also seen with other strains of P. stutzeri, and with strains of other Pseudomonas species that normally show no such activity (Table 2). Control pseudomonad strains containing only the cosmid vector, E. coli, or Rizobium meliloti harboring pT31, showed a Pdt⁻ phenotype (Table 2).

Sequence analysis of the pdt locus: the insert (SEQ ID NO:13) of pT31 was sequenced and analyzed in order to identify potential open reading frames (GenBank accession no. AF196567). Comparison of this sequence data with that of an 8.27 kb EcoRI fragment from strain KC described by Sepulveda-Torres et al. (GenBank accession No. AF149851) showed that these two EcoRI fragments were the same. Sepulveda-Torres et al. obtained the sequence after cloning from mutants lacking CCl₄ transformation activity as a result of mutagenesis by a specialized transposon (Sepulveda-Torres et al. “Generation and initial characterization of Pseudomonas stutzeri KC mutants with impaired ability to degrade carbon tetrachloride” Arch. Microbiol, 171:424-429 (1999)). To assess the function of various portions of the pT31 insert (SEQ ID NO:13) in Pdt complementation, pT31 was mutagenized using mini-Tn5 constructs (mini-Tn5 lacZ1, mini-Tn5 phoA (DeLorenzo et al. “Mini-Tn5 transposon derivatives for insertion mutagenesis, promoter probing, and chromosomal insertion of cloned DNA in Gram-negative eubacteria” J. Bacteriol. 172:6568-6572 (1990))). After mapping these insertions, their effect on PDTC production by strain CTN1 was measured (FIG. 1, Table 1). Although some open reading frames were not disrupted by any of the mapped insertions, an assessment of the extent of pT31 DNA required for complementation of the Pdt⁻ phenotype was afforded (FIG. 1).

Insertions that significantly influenced complementation activity were found in ORF-F (SEQ ID NO:14), ORF-J (SEQ ID NO:16), ORF-K (SEQ ID NO:1), and ORF-P (SEQ ID NO:5) (see FIG. 1). Homology searches showed that these ORFs encode proteins with significant similarities with MoeB/MoeZ sulfurylases, an AMP ligase, a receptor protein, and a methyltransferase, respectively (see Table 3).

TABLE 3
Putative open reading frames (RFs) encoded within
the pT31 insert (SEQ ID NO: 13).

| ORF/ | | | | | | |
|---|---|---|---|---|---|---|
| SEQ ID | Start | Ending | | | | |
| NO: | Base | Base | AA | kDa | pl | Homology |
| A/30 | 1 | 360 | >119 | >13.8 | NA | Similar to acyl-CoA synthase from |
| | | | | | | Mycobacterium bovis (AAB52538); |
| | | | | | | BLOSUM62 expect = 5 × 10⁻¹⁰, 36% |
| | | | | | | identities and 53% positives over 121 aa |
| | | | | | | overlap. |
| B/22 | 158 | 1099 | 313 | 34.2 | 10.39 | Probable thioesterase. Similar to |
| | | | | | | gramicidin S biosynthesis GRST protein |
| | | | | | | from Brevibacillus brevis (P14686); |
| | | | | | | BLOSUM62 expect = 3 × 10⁻²³, 35% |
| | | | | | | identities and 49% positives over 188 aa |
| | | | | | | overlap. |
| C/7 | 4066 | 3353 | 235 | 26.2 | 9.88 | Probable transcriptional activator. Similar |
| | | | | | | to XylS/AraC transcriptional activator from |
| | | | | | | Salmonella typhimurium (3094022); |
| | | | | | | BLOSUM62 expect = 2 × 10⁻⁹, 31% |
| | | | | | | identities and 50% positives over 107 aa |
| | | | | | | overlap. Has 25% identities and 50% |
| | | | | | | positives to 40 aa AraC bacterial regulatory |
| | | | | | | protein family protein signature (PROSITE |
| | | | | | | PS00041). |
| D/24 | 4486 | 5052 | 188 | 20.1 | 8.64 | No significant homology. |
| E/26 | 5187 | 6302 | 371 | 40.3 | 11.13 | No significant homology. Has 11 predicted |
| | | | | | | transmembrane domains. |
| F/14 | 6475 | 7650 | 391 | 43.7 | 4.88 | Possible sulfurylase. Similar to MoeZ from |
| | | | | | | Mycobacterium tuberculosis (CAB08310); |
| | | | | | | BLOSUM62 expect = 10⁻¹²², 57% |
| | | | | | | identities and 71% positives over 388 aa |
| | | | | | | overlap. Has a single predicted |
| | | | | | | transmembrane domain located between |
| | | | | | | residues 43 to 65. |
| G/9 | 7666 | 8076 | 136 | 15.6 | 6.68 | Similar to hypothetical 16.5 kd protein |
| | | | | | | RV1334 precursor from Mycobacterium |
| | | | | | | tuberculosis (Q10645); BLOSUM62 |
| | | | | | | expect = 8 × 10⁻³⁰, 47% identities and 67% |
| | | | | | | positives over 134 aa overlap. |
| H/11 | 8139 | 8411 | 90 | 9.7 | 5.61 | Similar to hypothetical protein from |
| | | | | | | Streptomyces coelicolor A3 (CAB50992); |
| | | | | | | BLOSUM62 expect = 6 × 10⁻²¹, 48% |
| | | | | | | identities and 74% positives over 90 aa |
| | | | | | | overlap. Lower homologies to MoaD |
| | | | | | | proteins. |
| I/18 | 8446 | 10332 | 628 | 67.5 | 6.98 | Similar to putative racemase from |
| | | | | | | Rhodococcus sp. (CAB55821); BLOSUM62 |
| | | | | | | expect = 2 × 10⁻²³, 30% identities and 44% |
| | | | | | | positives over 285 aa overlap. |
| J/16 | 10278 | 12023 | 581 | 62.8 | 5.51 | Probable AMP ligase. Similar to 2,3- |
| | | | | | | dihydroxybenzoate-AMP ligase from |
| | | | | | | Bacillus subtilis (P40871); BLOSUM62 |
| | | | | | | expect = 3 × 10⁻⁵⁰ , 28% identities and 45% |
| | | | | | | positives over 529 aa overlap. Has AMP |
| | | | | | | binding motif between residues 211-222. |
| K/1 | 11974 | 14037 | 687 | 75.3 | 5.93 | Probable receptor precursor. Similar to |
| | | | | | | FyuA precursor from Escherichia coli |
| | | | | | | (CAA84488); BLOSUM62 expect = |
| | | | | | | 2 × 10⁻²⁹, 25% identities and 40% positives |
| | | | | | | over 586 aa overlap. Has 25 aa signal |
| | | | | | | peptide located at amino terminus. Has |
| | | | | | | TonB-dependent receptor protein signature |
| | | | | | | (PROSITE PS00430). |
| L/28 | 14069 | 16300 | 743 | 83.3 | 7.50 | No significant homologies. |
| M/30 | 16716 | 19010 | 764 | 83.0 | 6.09 | Probable aminotransferase. Similar to |
| | | | | | | SC6A5.18 from Streptomyces coelicolor |
| | | | | | | (CAB39702); BLOSUM62 expect = |
| | | | | | | 4 × 10⁻⁷⁰, 34% identities and 50% positives |
| | | | | | | over 467 aa overlap. |
| N/3 | 19073 | 20251 | 392 | 40.6 | 10.72 | Probable ABC transporter. Similar to YycB |
| | | | | | | from Bacillus subtillis (CAB16085); |
| | | | | | | BLOSUM62 expect = 5 × 10⁻⁹, 24% |
| | | | | | | identities and 41% positives over 325 aa |
| | | | | | | overlap. |
| O/32 | 23041 | 21500 | 513 | 57.4 | 8.78 | Probable acyl-CoA dehydrogenase. Similar |
| | | | | | | to DR0922 from Deinococcus radiodurans; |
| | | | | | | BLOSUM62 expect = 1 × 10⁻⁷⁰, 38% |
| | | | | | | identities and 58% positives over 386 aa |
| | | | | | | overlap. |
| P/5 | 23969 | 22914 | 351 | 38.2 | 5.82 | Probable methyltransferase. Similar to |
| | | | | | | hydroxyneurosporene |
| | | | | | | methyltransferase (CrtF) from Rhodobacter |
| | | | | | | sphaeroides CRTF_RHOSH); BLOSUM62 |
| | | | | | | expect = 1 × 10⁻⁹, 30% identities and 40% |
| | | | | | | positives over 204 aa overlap. Has 67% |
| | | | | | | identities to S-adenosylmethionine- |
| | | | | | | dependent methyltransferase motifs (Kagan |
| | | | | | | and Clarke, 1994). |
| Q/34 | 25588 | 25746 | >53 | >6.4 | NA | Similar to predicted coding region AF1178 |
| | | | | | | (AAB90082) from Archaeoglobus fulgidus; |
| | | | | | | BLOSUM62 expect = 2 × 10⁻⁴, 45% |
| | | | | | | identities and 74% positives over 36 aa |
| | | | | | | overlap. |

Genbank accession numbers are in parentheses when provided.

The hypothetical MoeZ protein derives its name from its very high similarity to several molybdopterin synthase sulfarylase (MoeB) proteins. MoeB, along with the MoaD/MoaE heterodimer, molybopterin synthase, act to sulfurylate precursor Z of the molybdenum cofactor biosynthetic pathway (Rajagopalan, “Biosynthesis of the molybdenum cofactor.” In Escherichia coli and Salmonella typhimurium: Cellular and molecular biology. Neidhardt (ed). Washington D.C.: ASM Press, pp. 674-679 (1996)). The only mini-transposon insertion into ORF-F (SEQ ID NO:14) obtained in this study was pT-phoA1, a mini-Tn5 phoA insertion. This mini-transposon construct contains an alkaline phosphatase gene that is functionally expressed only when an in-frame translational fusion is created within an extracytoplasmic domain of a protein. A single predicted transmembrane segment is located at residues 43-65 of the 390 amino acid ORF-F product (SEQ ID NO:15). The phoA gene of pT-phoA1 was mapped to the carboxyterminal half of the protein (FIG. 1). In addition to the homology shared by the translated product of ORF-F (SEQ ID NO:15) and M. tuberculosis MoeZ, ORF-F (SEQ ID NO:14) also has nucleic acid homology to moeZ of M. tuberculosis (GenBank accession No. Z95120). The BLOSUM62 expect value for this homology was 9×10⁻¹⁵ with 80% identity over 194 bases (not shown). The AMP ligase, EntE, participates in the synthesis of the siderophore enterochelin by 2,3-dihydroxybenzoate adenylation and thioester coupling to pantetheinyl-EntB (Adams and Schumann “Cloning and mapping of the Bacillus subtilis locus homologous to Escherichia coli ent genes” Gene 133:119-121 (1993); Gehring et al. “Enterobactin biosynthesis in Escherichia coli: isochorismate lyase (EntB) is a bifunctional enzyme that is phosphopantetheinylated by EntD and then acylated by EntE using ATP and 2,3-dihydroxybenzoate” Biochemistry 36:8495-8503 (1997)). Both the mini-Tn5 insertions in ORF-J (SEQ ID NO:16) (pJS60 and pJS34) abolished PDTC production (Table 1). FyuA is an outer membrane protein involved in the uptake of ferric yersiniabactin in a TonB- and proton motive force-dependent fashion (Moeck and Coulton “TonB-dependent iron acquisition: mechanisms of siderophore-mediated active transport” Mol. Microbiol. 28:675-681 (1998); Pelludat et al. “The yersiniabactin biosynthetic gene cluster of Yersinia enterocolitica: organization and siderophore-dependent regulation” J. Bacteriol. 180:538-546 (1998); Rakin et al. “The pesticin receptor of Yersinia enterocolitica: a novel virulence factor with dual function” Mol. Microbiol. 13:253-263 (1994)). A variable effect of transposon disruption of ORF-K (SEQ ID NO:1) was seen, since one insertion (pJS59) resulted in a loss of PDTC production while the other (pJS52) did not (Table 1). A disruption of ORF-P (SEQ ID NO:5), the methyltransferase homolog (pJS40, Table 1, FIG. 1), resulted in low but detectable PDTC production. Deletion of ORF-P (SEQ ID NO:5) and a portion of ORF-O (SEQ ID NO:32) (pM22, FIG. 1) resulted in reduced CCl₄ transformation activity and low levels of PDTC production. ORF-P (SEQ ID NO:5) is transcribed in the opposite orientation to the other 3 ORFs shown experimentally to be required for normal PDTC production. The deletion introduced by construction of pJS68 removed a portion of ORF-N (SEQ ID NO:3) in addition to sequences truncated in pM22. This resulted in a loss of detectable PDTC production (FIG. 1, Table 1).

ORFs that were disrupted by mini-transposon insertions without significant effect on measured PDTC concentrations were ORF-C (SEQ ID NO:7), ORF-D (SEQ ID NO:24), ORF-E (SEQ ID NO:26), ORF-L (SEQ ID NO:28), and ORF-M (SEQ ID NO:30). ORFs that were not mutagenized by mini-transposon insertions were ORF-A (SEQ ID NO:20), ORF-B (SEQ ID NO:22), ORF-G (SEQ ID NO:9), ORF-H (SEQ ID NO:11), ORF-I (SEQ ID NO:18), ORF-N (SEQ ID NO:3), ORF-O (SEQ ID NO:32) and ORF-Q (SEQ ID NO:34). The coding regions of ORF-A (SEQ ID NO:20) and ORF-Q (SEQ ID NO:34) are incomplete in pT31 (Table 3, FIG. 1).

While not wishing to be bound by theory, the foregoing presumed functions of the proteins encoded by the open reading frames within SEQ ID NO:13, and the results of the foregoing transposon insertion experiments, suggest a biosynthetic pathway for PDTC as well as auxiliary functions for export and uptake. The (thiocarboxylate) groups may be generated by sulfurylation of a carboxylic acid precursor (by the ORF-F/MoeZ homolog and possibly ORF-G (SEQ ID NO:9) and H (SEQ ID NO:11) as accessory subunits) that has been activated by adenylation (by the ORF-J (SEQ ID NO:16) product/Ent E homolog). A novel aspect of this proposed synthesis pathway is that the sulfur atom remains bonded to the carbonyl carbon, whereas nonribosomal peptide synthase reactions using the thiotemplate mechanism (e.g., Ent B/G) effect substitution alpha to the carbonyl carbon. The finding that an ORF-F (SEQ ID NO:15)-phoA fusion produces active alkaline phosphatase and therefore is localized to a membrane suggests that PDTC biosynthesis occurs in proximity to the cytoplasmic membrane. Little is known regarding siderophore export but others have found evidence of membrane association of siderophore biosynthetic enzymes (Hantash and Earhart “Membrane association of the Escherichia coli enterobactin synthase proteins EntB/G, EntE, and EntF” J. Bacteriol. 182:1768-1773 (2000)). A potential exporter was identified in the ORF-N gene (SEQ ID NO:3), which shows homology with ABC transporters. Other evidence for ABC transporter involvement in siderophore export was found in the exochelin system of Mycobacterium smegmatis (Zhu et al. “Exochelin genes in Mycobacterium smegmatis: identification of an ABC transporter and two non-ribosomal peptide synthetase genes” Mol. Microbiol. 29:629-639 (1998)). Receptor-mediated uptake of PDTC-transition metal complexes is suggested by the ORF-K gene (SEQ ID NO:1). The apparent involvement of this gene product in PDTC biosynthesis or regulation is novel among siderophore regulatory networks. Other membrane receptors have been identified with autoregulatory functions (Venturi et al. “Gene regulation of siderophore-mediated iron acquisition in Pseudomonas: not only the Fur repressor” Mol. Microbiol. 17:603-610 (1995)), but no effects on siderophore biosynthetic genes were noted.

In view of the foregoing, it is therefore a further aspect of the present invention to provide isolated nucleic acid molecules that are at least 70% identical (such as at least 80% identical, or at least 90% identical, or at least 95% identical, or at least 99% identical) to any one of the nucleic acid molecules consisting of the nucleic acid sequences set forth in SEQ ID NO:1, SEQ ID NO:3, SEQ ID NO:5, SEQ ID NO:7, SEQ ID NO:13, SEQ ID NO:20, SEQ ID NO:22, SEQ ID NO:24, SEQ ID NO:26, SEQ ID NO:28, SEQ ID NO:30, SEQ ID NO:32 and SEQ ID NO:34.

It is a further aspect of the present invention to provide isolated protein molecules that are at least 70% identical (such as at least 80% identical, or at least 90% identical, or at least 95% identical, or at least 99% identical) to any one of the protein molecules consisting of the amino acid sequences set forth in SEQ ID NO:2, SEQ ID NO:4, SEQ ID NO:6, SEQ ID NO:8, SEQ ID NO:21, SEQ ID NO:23, SEQ ID NO:25, SEQ ID NO:27, SEQ ID NO:29, SEQ ID NO:31, SEQ ID NO:33 and SEQ ID NO:35.

It is yet a further aspect of the present invention to provide isolated nucleic acid molecules that hybridize to a nucleic acid molecule consisting of a nucleic acid sequence selected from the group of nucleic acid sequences consisting of SEQ ID NO:1, SEQ ID NO:3, SEQ ED NO:5, SEQ ID NO:7, SEQ ID NO:13, SEQ ID NO:20, SEQ ID NO:22, SEQ ID NO:24, SEQ ID NO:26, SEQ ID NO:28, SEQ ID NO:30, SEQ ID NO:32 and SEQ ID NO:34, under conditions of 1×SSC at 60° C. for 20 minutes, or to the complement of any one of SEQ ID NO:1, SEQ ID NO:3, SEQ ID NO:5, SEQ ID NO:7, SEQ ID NO:13, SEQ ID NO:20, SEQ ID NO:22, SEQ ID NO:24, SEQ ID NO:26, SEQ ID NO:28, SEQ ID NO:30, SEQ ID NO:32 and SEQ ID NO:34, under conditions of 1×SSC at 60° C. for 20 minutes.

### Example 2

This example sets forth the experimental materials and techniques used to generate the data set forth in Example 1.

Bacterial strains and culture conditions: Bacteria used in this study are listed in Table 4.

TABLE 4
Bacteria and plasmids used in this study

| Strain/Plasmid | GENOTYPE | SOURCE/REFERENCE |
|---|---|---|
| E. coli DH5α | F⁻end A1 hsdR17(r_{K}⁻m_{K}⁺) supE44 thi-1 | Gibco BRL |
| | recA1 gyrA relA1 Φ80dlacZΔM15 | |
| | Δ(lacZYA-argF)U169 | |
| E. coli S17-1 | Sm^{r} chromosomal RP4 integrant | S. Minnich, University of |
| | (mob⁺) | Idaho (Simon et al., 1983) |
| E. coli SM10(λ | Km^{r}, thi-1, thr, leu, tonA, lacY, supE, | K. Timmis, GBF, |
| pir) | recA::RP4-2-Tc::Mu, λpir | Braunschweig |
| E. coli Top-10 | F⁻, mcrAΔ(mrr-hsd/RMS-mcrBC), | Invitrogen Corp. |
| | Φ80 lacZΔM15, ΔlacX74deoR, | |
| | recA1, araD139Δ(ara-leu)7697, ga/U, | |
| | galK, rpsL, (Str^{r}) endA, nupG | |
| E. coli HB101 | (Sm^{r}) recA, thi, pro, leu, hsdR-M | |
| P. stutzeri KC | Wild type, aquifer isolate | C. Criddle, Stanford |
| | | University (Criddle et al., |
| | | 1990) |
| P. stutzeri CTN1 | Spontaneous derivative of strain KC | This study |
| P. stutzeri 17588 | Wild type, clinical isolate (type strain) | ATCC* |
| P. stutzeri 14405 | Wild type, marine isolate | ATCC |
| (ZoBell) | | |
| P. stutzeri 11607 | Wild type, clinical isolate | ATCC |
| P. fluorescens | Wild type soil isolate | F. O'Gara, University of |
| F113 | | Cork, Ireland |
| P. aeruginosa | Wild-type | N. Schiller, University of |
| PAO1 | | California, Riverside |
| P. putida | Wild type | DSMZ** |
| DSM3601 | | |
| P. putida mt-2 | Wild-type toluene degrader | University of Idaho culture |
| | | collection, (Williams and |
| | | Murray, 1974) |
| R. meliloti 102f34 | Wild-type | G. Ditta, University of |
| | | California, San Diego |
| Plasmid | Ap^{r} | Stratagene, La Jolla, Calif. |
| pBluescriptSK+ | | |
| Plasmid pRK311 | Tc^{r} broad host range cosmid (mob⁻ tra⁺) | M. Kahn, Washington |
| | | State University (Ditta |
| | | et al., 1985) |
| Plasmid pT31 | Pdt⁺ | pRK311 carrying ˜25.5 kb |
| | | insert of P. stutzeri strain |
| | | KC genomic DNA, this |
| | | study |
| Plasmid pUT-Km | Amp^{r}, Km^{r}, delivery plasmid for mini- | K. Timmis |
| mini-Tn5 lacZ1 | Tn5 lacZ1 | |
| Plasmid pUT-Km | Amp^{r}, Km^{r}, delivery plasmid for mini- | K. Timmis |
| mini-Tn5 phoA | Tn5 phoA | |
| Plasmid pRK2073 | Conjugation plasmid | G. Ditta |

*ATCC = American Type Culture Collection, Rockville, MD
**DSMZ = Deutsche Sammlung fur Mikroorganismen und Zellkulturen GmbH, Braunschweig, Germany

For routine growth of E. coli, LB medium was used (Sambrook et al. Molecular cloning: A laboratory manual Cold Spring Harbor, N.Y.: Cold Spring Harbor Laboratory Press (1989)). For testing CCl₄ transformation activity, M9 minimal medium with glucose (Sambrook et al. Molecular cloning: A laboratory manual. Cold Spring Harbor, N.Y.: Cold Spring Harbor Laboratory Press (1989)) and supplemented with 0.1 mM L-leucine and 5 nM CuCl₂ was used. Antibiotics used were from Sigma (St. Louis, Mo.) at the following concentrations: tetracycline (Tc, 7.5 μg/mL), ampicillin (Ap, 50 μg/mL), kanamycin (Km, 75 μg/ml), spectinomycin (Sp, 25 μg/ml), and nalidixic acid (Nal, 15 μg/ml). Substrate utilization tests employed Biolog™ plates (Biolog Inc., Hayward, Calif.). P. stutzeri were maintained on tryptic soy agar. Isolation streaks used for maintenance of strain KC contained <300 isolated colonies. A single colony was selected at random for transfer to fresh medium at approximately monthly intervals for this purpose. Stocks were initiated from frozen (−80° C., 15% glycerol) aliquots semi-annually to annually to avoid selection of strains evolved on laboratory medium. CCl₄ transformation was assessed using HM-acetate medium (Lewis and Crawford “Physiological factors affecting carbon tetrachloride dehalogenation by denitrifying bacterium Pseudomonas sp. strain KC” Appl. Environ. Microbiol. 59:1635-1641 (1993)) or DRM, which contained the following (per liter): K₂HPO₄, 6 g; sodium acetate, 2 g; sodium nitrate, 0.5 g; ammonium chloride, 1 g; adjusted to pH 7.8 and autoclaved. After cooling, 1 mL of 1M MgSO₄, 10 mL of Ca(NO₃)₂, and 50 μL of 0.1 mM CuCl₂ were added from sterile stock solutions. P. putida was grown on minimal succinate medium (SM) which contained: K₂HPO₄, 6 g; KH₂PO₄, 3 g; (NH₄)₂SO₄, 1 g; MgSO₄.7H₂O, 0.2 g; succinic acid, 4 g; pH was adjusted to 7.6 with NaOH (Meyer and Abdallah, 1978). Cultures (100 mL) were inoculated from aerobically-grown overnight cultures (1% v/v) and grown in 160 mL serum bottles stoppered with Teflon mininert valves (Pierce, Rockford, Ill.). CCl₄ was added from methanolic stock solutions. Cultures were incubated inverted, with rotary shaking at 150 rpm in a 25° C. incubator. Growth was monitored by protein assay using the bicinchoninic acid method (Pierce) performed on 1 mL samples either precipitated with 1 M trichloroacetic acid and digested at 100° C. for 30 min. in 1 M NaOH or treated with 100 μL toluene to burst the cells.

Analytical techniques: CCl₄ concentrations were determined by headspace gas chromatography with an electron capture detector as described previously (Lewis and Crawford “Physiological factors affecting carbon tetrachloride dehalogenation by denitrifying bacterium Pseudomonas sp. strain KC” Appl. Environ. Microbiol. 59:1635-1641 (1993)). Samples (0.1 mL) were taken with a 1 mL gas tight syringe (Hamilton, Reno, Nev.) and placed in 10 mL headspace autosampler vials. PDTC was determined spectrophotometrically with a Hewlett Packard 8453 diode array spectrophotometer (Budzikiewicz et al. “Weitere aus dem Kulturmedium von Pseudomonas putida isolierte Pyridinderivate—Genuine Metaboliten oder Artefackte?” Z. Naturforsch 38b:516-520 (1983)).

Recombinant DNA techniques: A genomic library of strain KC was constructed using DNA partially digested with Sau3A and size fractionated on a 10-40% sucrose density gradient (Sambrook et al. Molecular cloning: A laboratory manual. Cold Spring Harbor, N.Y.: Cold Spring Harbor Laboratory Press (1989)). Size-fractionated DNA (>approx. 20 kb) was ligated into pRK311 that had been treated with BamHI and shrimp alkaline phosphatase (Gibco). Ligation products were packaged into phage particles using Gigapack II XL extracts (Stratagene). These were used to infect E. coli DH5α, for titer determination and insert efficiency on X-Gal/IPTG, and E. coli S17-1 as the library host. The average insert size was determined to be 28 kb among 10 random picks. One thousand colonies were picked for storage in 15% glycerol and used to prepare colony lifts on nylon membranes (MagnaLift, Micron Separations Inc., Westboro, Mass.,). Cosmids were transferred into pseudomonads or other strains of E. coli by conjugation, performed by the patch mating technique (De Feyter and Gabriel “Use of cloned DNA methylase genes to increase the frequency of transfer of foreign genes into Xanthomonas cainpestris pv. Malvacearum” J. Bacteriol. 173:6421-6427 (1991)) on TSA plates (for pseudomonads) or LB plates (for E. coli). Matings were conducted for 3-6 h at 30° C. for pseudomonads or for 1-3 h at 37° C. for E. coli. Southern transfer of DNA restriction digests to nylon membranes (Hybond N, Amersham, UK) was done with a vacuum blotting apparatus (Hoefer Scientific TransVac, San Francisco, Calif.) or by capillary transfer (Sambrook et al. Molecular cloning: A laboratory manual. Cold Spring Harbor, N.Y.: Cold Spring Harbor Laboratory Press (1989)). Probes for hybridization were synthesized using the RadPrime kit from Gibco and α³²P-dCTP (NEN, Boston, Mass.) or the NEBlot non-radioactive labeling kit (New England Biolabs, Beverly, Mass.). Pad and I-CeuI restriction endonucleases were from New England Biolabs; all others were from Gibco BRL.

16S rDNA sequences were determined by PCR amplification from genomic DNA templates and primers designed from published sequences. The products were cloned into pBluescript KS+ and three individual clones from each product were sequenced using M13 forward and reverse primers and the Sequitherm EXCEL™ II Long-Read™ DNA sequencing kit (Epicentre Technologies Corp., Madison, Wis.) and a Li-Cor automated sequencer (Li-Cor Inc., Lincoln, Nebr.). The strain KC nosZ and recA genes and the chromosomal origin of replication (ori) of P. stutzeri strain KC were obtained by PCR amplification and cloning into TOPO TA (Invitrogen, Carlsbad, Calif.). PCR reactions used primers designed from published sequences (GenBank accession numbers M22628, L12684, and M30125 respectively). Identities of the cloned products were confirmed by sequencing and alignment searching using BLAST programs (Altschul et al. “Gapped BLAST and PSI-BLAST: a new generation of protein database search programs” Nucleic Acids Res, 25:3389-3402 (1997)).

Pulsed field gel electromhoresis: Agarose-embedded DNAs from P. stutzeri were prepared and digested with restriction enzymes as described by Römling et al. (“Bacterial genome analysis by pulsed field gel electrophoresis techniques” In Advances in Electrophoresis. Chrambach, Dunn, and Radola (eds.), Wenheim, Germany: Wiley-VCH, pp. 335-406 (1994)). PacI digests were done by first allowing the enzyme to diffuse into the agarose at 4° C. for 3-16 h before incubation at 37° C. for 2 h. The digests were electrophoresed using a CHEF DRII or DRIII apparatus (Bio-Rad, Richmond, Calif.). Gels were run at 14° C. using molecular biology grade agarose (International Biotechnologies Inc., New Haven, Conn.) in 0.5×TBE (Sambrook et al. Molecular cloning: A laboratory manual. Cold Spring Harbor, N.Y.: Cold Spring Harbor Laboratory Press (1989)) and an included angle of 120°. Agarose concentrations, voltages, and pulse lengths, optimized for particular experiments, are given in figure legends. Size standards used were Saccharomyces cerevisiae chromosomes, phage λ oligomers (New England Biolabs), λ HindIII digests (Sigma), and Hansenula wingei chromosomes (Bio-Rad).

DNA sequencing and sequence analysis: The insert of pT31 (SEQ ID NO:13) was sequenced by a combination of subcloning and primer walking. Dye termination reactions were processed on an ABI 377 sequencer (Perkin-Elmer, Foster City, Calif.). Assembly of sequence data was performed using OMIGA version 1.1 (Kramer 2000). From an initial set of potential open reading frames greater than 50 amino acids in length, 17 open reading frames were selected as potential coding regions by analysis of codon bias (Staden et al. “The Staden package” Meth. Mol. Biol. 2000 132:115-130 (2000)) (Table 3). Homology searches were conducted using BLASTP and BLASTN (Altschul et al. “Gapped BLAST and PSI-BLAST: a new generation of protein database search programs” Nucleic Acids Res, 25:3389-3402 (1997)). For identification of protein domains and signatures, PROSITE and BLOCKS databases were searched using the ExPASy proteomics server and NCBI's Impala search engine, respectively (Appel et al. “A new generation of information retrieval tools for biologists: the example of the ExPASy WWW server” Trends Biochem. 19:258-260 (1994); Henikoff and Henikoff “Protein family classification based on searching a database of blocks” Genomics 19:97-107 (1994)). Prediction of transmembrane regions of protein sequences was performed by the TMHMM program. Calculation of isoelectric points and molecular weights for putative protein sequences was performed by using the Wisconsin package (Butler “Sequence analysis using GCG” Meth. Biochem. Anal. 39:74-97 (1998)).

Transposon mutagenesis: Donor strain E. coli SM10 (pUT-Km mini-Tn5 lacZ1) or (pUT-Km mini-Tn5 phoA), helper strain E. coli HB101 (pRK2073), and recipient strain E. coli Top-10 (pT31) were grown overnight at 37° C. with antibiotic selection. 0.3 mL of each culture were combined and filtered through a 0.21 μm (pore size) filter (Supor®, Gelman Sciences, Ann Arbor, Mich.). Filters were placed on an LB plate and incubated for at least 8 hours at 37° C. Mating mixtures were then resuspended in sterile M9 medium (w/o carbon source). 0.1-0.5 mL of this suspension was spread on LB Km, Tc and grown at 37° C. overnight. The Ki^{r} Tc^{r} E. coli were resuspended by rinsing the plates with M9 medium. Tc^{R}, Km^{R} plasmids in this suspension were recovered by “mating out” using DH5α as the recipient (3 h, 37° C.). DH5α (pT31-KmlacZ1) were amplified by plating on LB Km, Tc, Nal. For mini-Tn5 phoA, the Tc^{R}, Km^{R} plasmids were mated out with CTN1 and plated on M9 citrate Km, Tc and BCIP (5-bromo-6-chloro-3-indolyl Phosphate, Sigma). Among several hundred colonies, one showed an intense blue color after 10 days. The cosmid present in this transconjugant (pT-phoA1) was transferred to DH5α for DNA preparation. The location of mini-Tn5 insertions was determined by single-digests with EcoRI and XhoI (lacZ1) or EcoRI and BamHI (phoA) and separation by electrophoresis through 0.8% agarose.

### Example 3

Degradation of Carbon Tetrachloride by PDTC Metal Complexes

In this example, the degradation of carbon tetrachloride by PDTC metal complexes is described.

CCl₄ transformation assays. CCl₄ transformation assays were performed in 2 ml of 35 mM potassium phosphate buffer (KH₂PO₄/KOH, pH 7.7) prepared with glass-distilled deionized water and stored in an anaerobic chamber (Forma Scientific, atmosphere N₂:H₂:CO₂, 85:10:5) with 1 g of Chelex 100 chelating resin (Sigma Chemical Co., St. Louis, Mo.) per 100 ml. All glassware used for stock solutions or reactions was cleaned with aqua regia (conc. HCl:conc. HNO₃, 4:1, vol/vol). Reaction mixtures (2 ml) containing approximately 25 μM PDTC were prepared in the anaerobic chamber in 20-ml-headspace autosampler vials, and received a total of approximately 0.2 μmoles CCl₄. CCl₄ (Omnisolv, Merck) was added from a methanol stock solution (approx. 0.8%, vol/vol). Additions of CCl₄ were made with a 25-μl gas-tight syringe (Hamilton, Reno, Nev.) immediately before sealing with a Teflon-faced butyl rubber stopper (West Co., Phoenixville, Pa.) and aluminum crimp seal. Reactions were incubated at 25° C. in an inverted position in test tube racks for 72 hours unless otherwise noted.

Hydrogen sulfide was from Aldrich (Milwaukee, Wis.). Sodium sulfide (Na₂S×9H₂O) was from EM Science (Gibbstown, N.J.). Titanium(III) citrate was prepared in the anaerobic chamber from 20% Ti(III)Cl₃/HCl solution (Fisher), trisodium citrate, and sodium carbonate to give a final pH of 7.7 and a final concentration of 0.5M Ti. ¹³CCl₄ was from Cambridge Isotope Laboratories (Andover, Mass.) and ¹⁴CCl₄ was from DuPont NEN (Wilmington, Del.). CuCl₂, 99.999%, and FeCl₃×6H₂O, 98%, were from Aldrich. CoSO₄×7H₂O, 99%, was from Sigma. NiCl₂×6H₂O, 99.9999%, was from Fisher (Acros).

Bioassays. Pseudomonas stutzeri American Type Culture Collection strain 17588, known from previous work to have no significant CCl₄ transformation activity or PDTC biosynthetic capacity, was used in assays of CCl₄ transformation with PDTC. The organism was grown aerobically in a medium containing (per liter) dipotassium phosphate, 6 g; sodium acetate, 2 g; ammonium sulfate, 1 g; sodium nitrate, 0.5 g. The pH of the medium was adjusted to 7.7-7.9 with HCl before autoclaving, and, after cooling, calcium nitrate and magnesium sulfate were added from sterile stock solutions to final concentrations of 0.1 mM and 1 mM, respectively. Cultures were grown overnight at 30° C. Aliquots (1 ml) in 10 ml headspace autosampler vials were used in triplicate assays containing 50 μM PDTC and 30 nmoles CCl₄. These were incubated at 25° C., inverted, for 16 hours.

Synthesis of PDTC and its metal complexes. PDTC was synthesized by the method of Hildebrand et al., Phosphorus Sulfur, 16:361-364 (1983). Aqueous solutions were prepared by dissolving PDTCH₂ (5 mM) in anoxic 35 mM potassium phosphate buffer (pH 7.7), followed by filtration through 0.2-μ (pore size) membranes. The Cu—Cl and Cu—Br complexes were isolated as the tetrabutylammonium salts, as described for the synthesis of Pd—Br:PDTC complex (Espinet et al., Inorg. Chem. 33:2052-2055(1994)). Elemental analysis of the Cu—Br complex gave (theoretical in parentheses): C, 47.5% (47.37); H, 6.62% (6.74); N, 4.81% (4.80); S, 11.17% (11.00).

Analytical methods: Gas chromatography. For assays to determine the effectiveness of various metal ions in promoting CCl₄ dechlorination, determinations of CCl₄ were made by gas chromatography/mass spectroscopy (Thermoquest Trace GC/Trace MS, Thermo Separation Products, San Jose, Calif.). For optimal linearity of responses in the range of concentrations encountered, CCl₄ was detected by single ion monitoring (SIM) of the ion m/z 82; CHCl₃, m/z 83; CS₂, m/z 76 or 78. One milliliter injections were made by a headspace autosampler (HS2000) following a 10 minute conditioning cycle at 70° C. with shaking. The column used was a Supel-Q PLOT (30 m×0.32 mm, Supelco, Bellefonte, Pa.). Carbonyl sulfide (COS) and carbon disulfide were analyzed by headspace gas chromatography with a Hewlett Packard 6890 GC, a PoraPLOT Q column (Chrompack, Middelburg, The Netherlands) and a 5973 mass selective detector and integration of the total ion chromatograms (TIC—20-150 Daltons). Injections were made using a 7693 headspace autosampler. Vials were equilibrated to 70° C. for 10 min with shaking before injection to a 1-ml sample loop. The concentration of CCl₄ in bacterial cultures was measured using a Hewlett Packard (Avondale, Pa.) 5890 gas chromatograph equipped with an electron capture detector and a 19395 headspace autosampler. Standards were prepared by additions of analytes (CCl₄, CHCl₃, CS₂) from stock methanol solutions.

Radiotracer analysis. ¹⁴C-labeled CCl₄ was used to determine mass balances with a nitrogen-purging manifold, employing organic traps, base traps, and scintillation counting. This method gave 100±7% recovery of ¹⁴CO₂ from NaH¹⁴CO₃ and 93.2±1.1% for ¹⁴CCl₄.

Electrospray MS. The non-volatile species present in reaction mixtures was analyzed by negative or positive electrospray ionization tandem mass spectrometry (Quattro II, Micromass Ltd., UK). Concentrated reactions used for mass spectral identification of products were 2 mM Cu:PDTC and approximately 20 μl CCl₄ per ml of reaction volume in DMF:H₂O, 1:1. Samples were delivered into the source at a flow rate of 5 μL/min using a syringe pump (Harvard Apparatus, South Natick, Mass.). A potential of 2.5-3 kV was applied to the electrospray needle. The sample cone was kept at an average of 15 V. The counter electrode, skimmer, and RF lens potentials were tuned to maximize the ion beam for the given solvent. Resolution of the detector was 15,000 and source temperature was kept constant at 80° C. The instrument was calibrated using a polyethylene glycol solution. All spectra were an average of 10-15 scans.

Liquid chromatography. Chloride was measured using a Dionex 2010i ion chromatograph equipped with an AS4a column (Dionex, Sunnyvale, Calif.), Na₂CO₃/NaHCO₃ eluent at 2 ml/min, and suppressed conductivity detection.

Dipicolinic acid (pyridine-2,6-dicarboxylic acid) was measured on a Thermo Separations Products HPLC, SpectraSystem P2000 with an AS3000 autosampler and 10 μl injections. The column was a 4.6×250 mm 5μ Hypersil BDS C18. Analytes were eluted using 25 mM sodium phosphate pH 7.0, 5 mM tetrabutylammonium phosphate (TBAP) {A} and acetonitrile, 5 mM TBAP {B}. A gradient was generated by pumping 1 ml/min of 95% A and increasing to 65% B over 15 min. Detection was by UV absorption at 260 nm with spectral scanning using a UV6000LP photodiode array detector.

Electron Paramagnetic Resonance. EPR spectra were recorded at X-band frequencies using a Bruker ESP300E spectrometer (Bruker Instruments, Billerica, Mass.). Samples were loaded in a quartz flat cell (Wilmad Glass Co., Buena, N.J.) in an anaerobic chamber and sealed with Teflon stoppers and parafilm prior to transferring into the instrument sample cavity. Spectra were collected at room temperature as follows: microwave frequency 9.68 GHz, microwave power 20 mW, modulation frequency 100 KHz, modulation amplitude 1.0 G, conversion time 164 ms, time constant 328 ms, number of scans averaged 32, number of data points 4096, sweep width 75 gauss, sweep time per scan 671 s. The spin trap α-phenyl-tert-butyl nitrone (PBN, Sigma) was purified by vacuum sublimation prior to use. Trapping experiments were performed in the presence of 100 mM PBN.

The following summarizes the effect of transition metal ions on PDTC-mediated carbon tetrachloride transformation.

No added reductant. The metal ions tested included those known to have effects upon dechlorination by strain KC (Fe, Co, Cu) and/or known to form complexes with biological ligands that are active for catalytic dechlorination (Fe, Co, Ni). Without added transition metal, no significant CCl₄ transformation was seen (see Table 5 below). Copper was the only metal found to have a significant stimulatory effect on CCl₄ transformation. No inhibitory effects could be observed in these assays since the control (no metal addition) showed no significant transformation; however, CoSO₄ (13 μM) addition to PDTC prior to CuCl₂ addition (13 μM) led to no significant CCl₄ transformation. FeCl₃ addition (50 μM) prior to CuCl₂ addition did not prevent transformation.

Bacterial cells and chemical reducing agents. Conditions known to show PDTC-dependent CCl₄ transformation at trace copper concentrations were used to assess inhibitory effects of transition metals. These included suspensions of non-PDTC-producing bacterial cells, and chemical reductants. These conditions were not subject to inhibition of PDTC biosynthesis and therefore should detect only direct chemical effects. Variables introduced by the use of sulfide and Ti(III) as reductants are their respective redox potentials, which affect their ability to reduce the transition metals in complex with PDTC. For example Ti(III) (Eₒ′=−0.480V) has been shown to reduce Co(III) in cyanocobalamin to Co(I) (10, 14), but thiols such as dithiothreitol (Eₒ′=−0.332V) cannot reduce cyanocobalamin beyond Co(H) (11). Bacterial cells, sulfide, or Ti(III) citrate affected significant CCl₄ transformation in combination with PDTC without added transition metals (see Table 5 below). With bacterial cells or sodium sulfide, only cobalt addition resulted in an inhibition of CCl₄ transformation (see Table 5 below). With Ti(III) citrate, this effect was less pronounced and iron addition also resulted in decreased transformation.

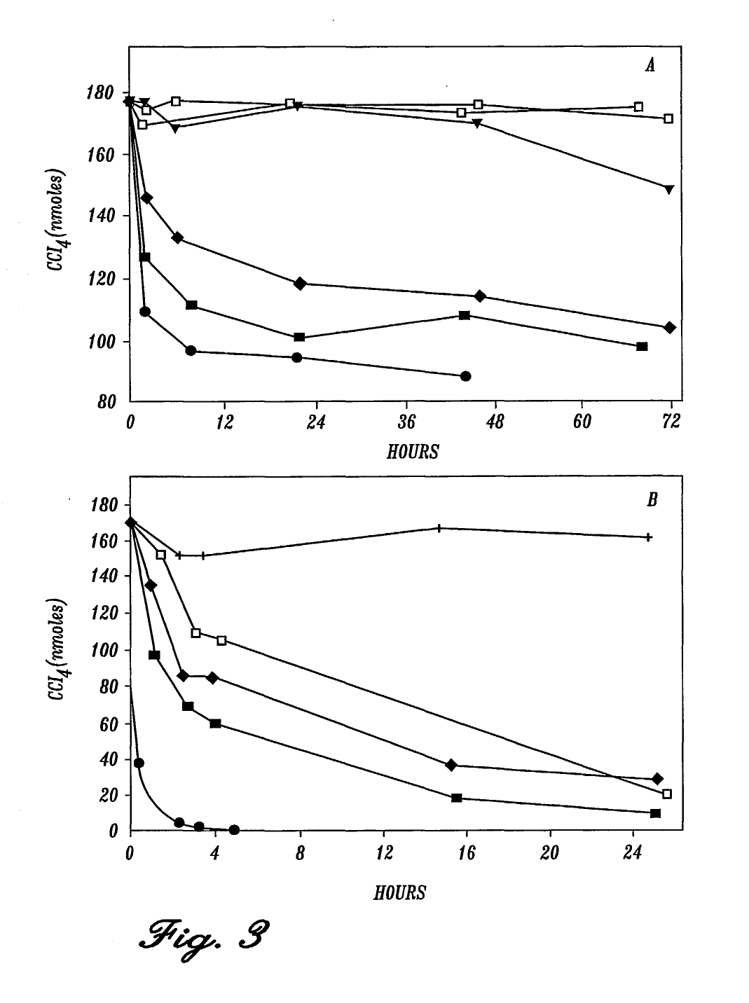

The fact that cobalt effectively inhibited transformation and that copper stimulated it indicated the importance of metal ligation for dechlorination by PDTC. An explanation for these effects may be that copper was the only metal among those tested capable of promoting reaction between CCl₄ and PDTC. This requirement may have been met by very low concentrations of copper, but cobalt effectively excluded copper from the ligand. Atomic absorption spectroscopy determinations indicated that there was approximately 0.26 mmoles of copper per mole of PDTC, and therefore at least 6 nM copper was present in all reactions including PDTC. Time course experiments were performed to determine the effect of copper concentration on CCl₄ transformation, with and without reductants. The data are shown in FIG. 3. The kinetics of CCl₄ transformation were very rapid at 0.5 moles Cu/mole PDTC, and slower at the lower copper concentrations. Rate constants could not be determined with the limited data, but by simple observation, the extent of transformation at a given time point was dependent on copper concentration. When sulfide was present this effect was evident over an 8-hour time course with additions of only 75 nM CuCl₂. In contrast, when no reductant was added 188 nM CuCl₂ did not affect significant transformation over a 42-hour time period (FIG. 3). A differential response to copper imparted by the reductant was clearly evident in these data. This effect explains CCl₄ transformation by PDTC without added copper as due to the presence of copper contamination.

Stoichiometry of PDTC-dependent CCl₄ transformation and quantitation of hydrogenolysis products. The data described above were useful in identifying which metals had direct effects on dechlorination by PDTC, but did not indicate which conditions were most representative of dechlorination by strain KC. This determination required a more detailed description of the transformation products. Catalytic dechlorination agents such as corrinoids or hematin show a large proportion of hydrogenolytic products in the presence of excess reductant. The CCl₄ transformation seen with strain KC is characterized by very few hydrogenolytic products, but significant sulfur substitution. Chloroform and carbon disulfide were measured in the experiments of Table 5. Those data are given, along with CCl₄ turnover calculations, in Table 6. A stoichiometry of approximately two moles of CCl₄ per mole of PDTC was obtained when CuCl₂ was added without added reducing agents (see Table 5 below). These conditions also resulted in low amounts of chloroform and a significant amount of carbon disulfide. Sulfide addition led to much higher turnover of CCl₄ per mole of PDTC, carbon disulfide as a major product, and increased chloroform. Titanium(III) citrate addition also led to higher turnover and chloroform production. A comparison of metal additions showed that the products detected under conditions promoting transformation resembled those seen with strain KC more closely than products of hydrogenolysis catalysts, with the exception of cobalt/Ti(III) citrate.

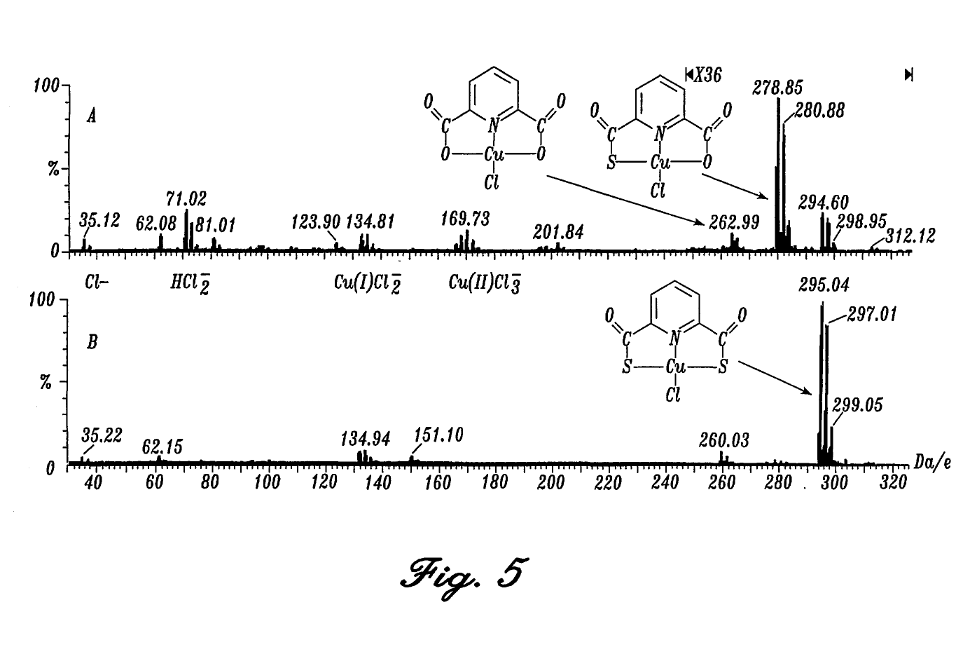

Structure of the Cu:PDTC complex. PDTC is known to form stable complexes with iron, cobalt and nickel. Hexacoordinate (i.e., coordinatively saturated) metal ion:PDTC complexes comprised of one metal atom and two PDTC ligands have been described for Fe(II, III), Ni(II), and Co(III) using X-ray diffraction spectroscopy. In these complexes the metal atom lies in the center of octahedrally arranged ligand atoms formed by two planar PDTC molecules arranged perpendicular to each other. Using negative ion electrospray mass spectrometry (ES⁻ MS), the following molecular ions were observed: [Fe(II)(PDTC)₂]⁻², m/z 225; [Fe(III):(PDTC)₂]⁻¹, m/z 450; [Co(PDTC)₂]⁻¹, m/z 453. Structures for Co[I] and Co[II] complexes with PDTC have not been determined. Palladium is known to form a planar tetracoordinate 1:1 complex with PDTC that can also include a halide ion. Cu(II) also forms a 1:1 complex with PDTC in which copper can be coordinated by a halide ion. The ES⁻ MS analysis of CuCl:PDTC showed molecular ions: m/z 295, [⁶³Cu³⁵Cl:PDTC]⁻¹, 100%; m/z 297, [⁶⁵Cu³⁵Cl+⁶³Cu³⁷Cl:PDTC]⁻¹, 84.45% ; m/z 299, [⁶⁵Cu³⁷Cl:PDTC]⁻¹, 18.85%, (example in FIG. 5B). Elemental analysis of the Cu(II)—Br:PDTC complex also confirmed 1:1 stoichiometry. EPR spectra of the Cu—Br:PDTC complex are typical of Cu(II) complexes. A Cu(II) oxidation state assignment is also supported by elemental analysis which indicates one tetrabutylammonium cation per Cu—Br:PDTC anion.

Structures of the relevant complexes indicated that metal ions known to be active in reductive dechlorination of CCl₄ in other coordination complexes (i.e., Fe(II), Co(II), and Ni(II) in heme, corrins, and F₄₃₀, respectively) were not active when complexed with PDTC due to steric and/or electronic effects imparted by the sulfur and nitrogen atoms surrounding the metal center. Ti(III) citrate was the only reductant used that is likely to produce Co(I), which occurs as a planar tetracoordinate complex in vitamin B₁₂. An inner-sphere electron transfer process has been described for dechlorination by Fe(II) porphyrins. The data from PDTC-transition metal complexes are consistent with such a process in that only when a 1:1 complex was demonstrated (Cu(II):PDTC) or predicted (Co(I):PDTC) was dechlorination activity observed. The data showing no dechlorination activity by iron, cobalt, or nickel complexes (i.e., without reductant or cells; Table 5) suggested a model whereby only PDTC complexes having an accessible metal atom are active. The Co(II) complex having the highest stability, followed by Cu(II), best explained the data. Cu(II) could displace Fe(III) or Ni(II) and the dechlorination activity seen in the presence of iron and nickel would likely have been due to contaminating copper.

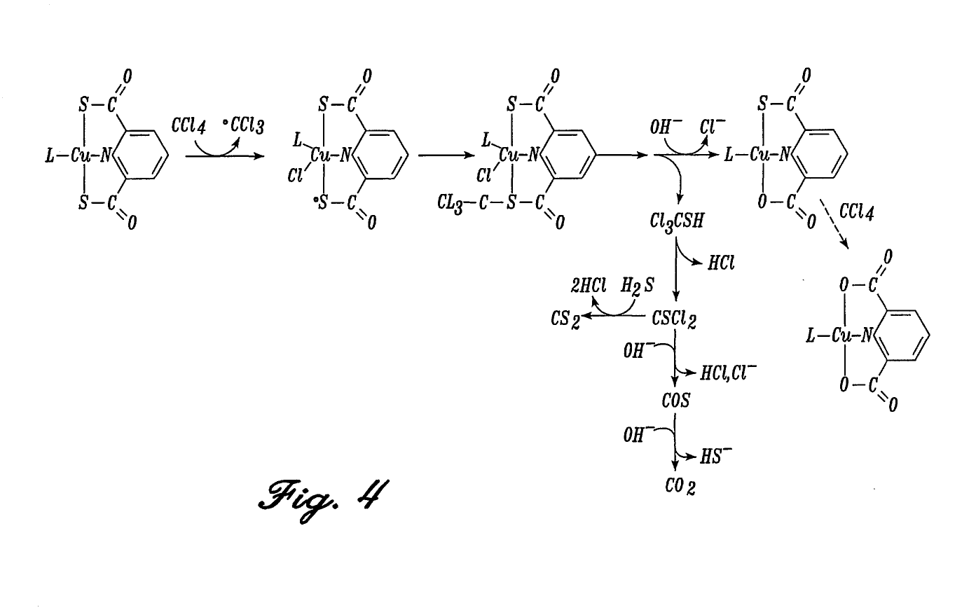

Dechlorination of CCl₄ by PDTC-transition metal complexes. To explain the observed products, a reaction pathway outlining events likely to occur after the initial encounter between CCl₄ and the metal atom has been formulated (see FIG. 4). The mechanism involves atom transfer and is reductive, with an initial one-electron transfer to CCl₄ from the Cu:PDTC complex. In this pathway CCl₄ is converted to CO₂ and HCl, and PDTC to DPA and H₂S. CS₂ is a byproduct. The critical difference between reduction by Co[I]:PDTC and the pathway of FIG. 4 would be that oxidation of Co[II] would not lead to oxidation of the neighboring sulfur atom. The oxidation of Cu:PDTC provides [Cu:PDTC]., whereas oxidation of Co(I):PDTC provides Co(II):PDTC. Evidence for oxidation of sulfur ligand atoms without net oxidation of a coordinated metal atom has been described. The oxidizing trichloromethyl radical formed by one-electron reduction of CCl₄ would be expected to have different fates under the two circumstances; with a radical in close proximity condensation of the two radicals to form a covalent bond would dominate. Without this proximal radical, reactions yielding chloroform via reduction and protonation or hydrogen abstraction are favored, explaining the higher chloroform yield with Co/Ti(III) in Table 6. The presence of reducing agents increased the likelihood of reduction of the radical and hydrogenolysis, a radical-scavenging effect. The reductants will likely reduce Cu(II) to Cu(I), which could allow some catalytic reduction of CCl₄ by the Cu:PDTC complex. An additional catalytic effect would be derived from replacement of the hydroxide in FIG. 5 with thiolate (HS⁻) as the attacking nucleophile. This would result in regeneration of the active agent, whereas hydrolysis would destroy an element of the structure required for activity by replacing sulfur with oxygen. These results are supported by the stoichiometry data of Table 6 in that the non sulfur-containing reductant, Ti(III) citrate, was less effective at increasing turnover than was sulfide.

Mass balance analysis. End product analyses were used to further assess how well the chemically defined reaction conditions represented CCl₄ transformation by strain KC. Mass balance determinations using ¹⁴CCl₄ radiotracer were used initially and showed that Cu:PDTC without added reducing agent gave a product profile consisting of approximately 65% ¹⁴CO₂, 15% non-volatile material, and 20% volatile organic-trapped products (see Table 7 below). Addition of Na₂S resulted in a substantial increase in the volatile organic-trapped product fraction and a decrease in ¹⁴CO₂ (see Table 7 below). Headspace GC/MS analysis detected carbon disulfide (CS₂) and chloroform (see Table 6 below), as well as COS as products likely to be trapped in the organic scintillant used. Those data indicated that the organic-trapped ¹⁴C was mostly ¹⁴CS₂. Using ¹³CCl₄ as a tracer confirmed that the carbon atoms of CS₂ and COS were derived from CCl₄ and not from rearrangements of PDTC. Less COS was seen when Na₂S was added. These data are consistent with predictions set forth in the pathway of FIG. 4; i.e., that CO₂ was derived from hydrolysis of thiophosgene via COS, and that the nucleophilic thiolate ion reacted with the electrophilic thiophosgene to give CS₂ at the expense of CO₂ and COS production. Comparing the data to those obtained from strain KC cultures, more CO₂ and organic-trapped products and fewer non-volatile products were seen with Cu:PDTC. These results can be explained by the presence of active nucleophiles other than HS⁻ in cultures versus the chemically-defined conditions.

Non-volatile product analysis. The non-volatile material remaining in concentrated reactions containing 2 mM Cu:PDTC and excess CCl₄ was analyzed by ES⁻ MS. Since sulfur-substitution was seen in reactions where PDTC was the only sulfur compound included, the presence of a de-sulfurylated PDTC derivative was anticipated. Reactions were performed in DMF:water to improve solubility of synthetic Cu:PDTC and facilitate ionization in electrospray MS. These reactions showed substantial bleaching of the green color of Cu:PDTC (λmax: 400 nm, 610 nm) as a result of CCl₄ addition, giving a pale blue solution (λmax: 333 nm, 710 nm) with the appearance of a brown precipitate. These results were observed whether the reactions were conducted anoxically or under an air headspace with air-equilibrated solvent. Mass spectra showed that the concentration of Cu:PDTC decreased, and that Cu:dipicolinate and an ion attributed to the partial hydrolysis product, picolinic acid-6-thiocarboxylic acid, appeared (see FIG. 5). When the reactions were performed in phosphate buffer (i.e., no DMF) with catalytic amounts of copper (1 mole %), dipicolinic acid was the major aromatic component seen by HPLC. Dipicolinic acid accounted for approximately 70% of the PDTC included in the reaction after 48 hours.

Evidence for radical intermediates. Some of the reaction products observed with Cu:PDTC/CCl₄ (CS₂, CO₂) have been predicted to arise from biotite/sulfide/CCl₄ mixtures through a radical substitution mechanism. The pathway of FIG. 4 also includes trichloromethyl radical as an intermediate. Oxygen is known to react with trichloromethyl radical at rates near diffusion limitation. The pathway proposed would predict that the presence of O₂ would divert a significant portion of the carbon flow away from sulfur-substitution products. The data of Tables 5 and 6 and FIG. 3 were obtained from anoxic conditions. When reactions were conducted under an air headspace with air-equilibrated buffer, carbon disulfide accounted for less than 0.4% of the CCl₄ transformed, whereas in a parallel experiment conducted anoxically, it accounted for 15.7% (13 μM CuCl₂, 26 μM PDTC, 4 hour incubation). The turnover of CCl₄ per PDTC in those experiments was 1.08±0.02 (mean±s.d., n=3) under air, and 1.45±0.08 anoxically. Thiols are subject to autoxidation, which can be catalyzed by transition metal ions. Solutions of PDTC (5 mM) with CuCl₂ (50 μM) showed an O₂-dependent degradation as evidenced by formation of a precipitate after several hours in air. Competition between O₂ and CCl₄ for oxidation of Cu:PDTC is therefore likely; however, it did not prevent dechlorination from occurring when exposure to CCl₄ was initiated shortly after exposure to O₂.

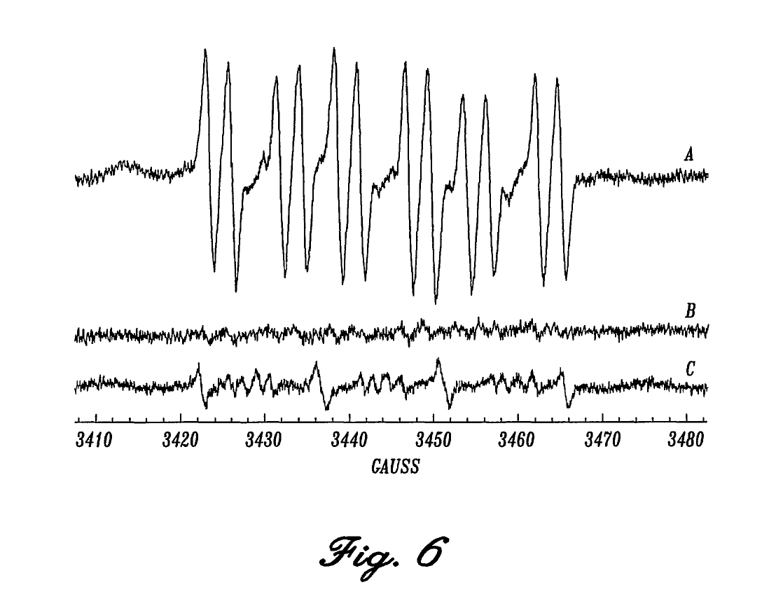

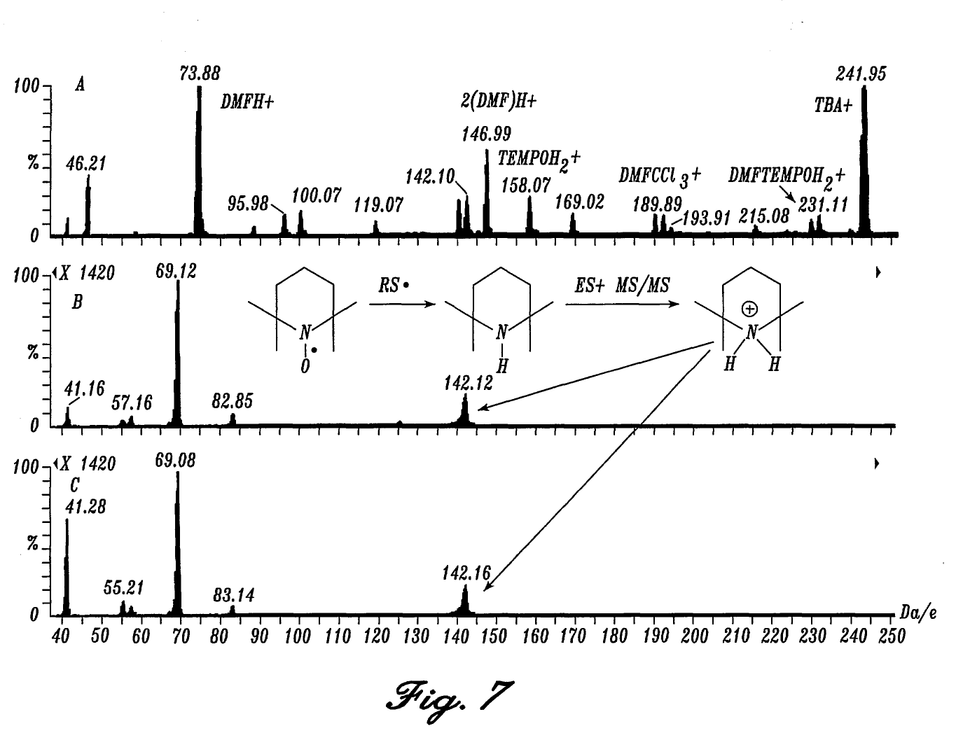

Additional experiments were conducted to characterize possible radical intermediates of the Cu:PDTC/CCl₄ reaction. Using EPR spectroscopy, reactions performed in phosphate buffer with catalytic amounts of CuCl₂ and excess PBN spin trap exhibited a resonance centered at g=2.008 with hyperfine coupling constants of A_{α}^{N} 15.3 G, and A_{β}^{H} 2.7 G. Nitroxyl radicals typically exhibit a basic triplet pattern due to nitrogen hyperfine (I=1) coupling to the radical. With additional hyperfine coupling to one hydrogen (I=½), as found in PBN trapped species, a triplet of doublets pattern is observed. To obtain definitive evidence for trapping of .CCl₃ it is convenient to introduce a further hyperfine signature through ¹³C labeling (I=½). A characteristic triplet of doublet of doublets pattern is then observed for PBN trapped .¹³CCl₃ as shown in FIG. 6. Measured hyperfine coupling constants of A₆₂ ^{13C} 8.4 G, A_{α}^{N} 15.3 G, and A_{β}^{H} 2.6 G were consistent with previously determined values for [¹³C trichloromethyl]-PBN spin adduct, definitively showing that trichloromethyl radical had arisen from reaction between CCl₄ and PDTC with copper. In light of the potent reducing ability of SH-containing species, we suspected that some of the PBN-trichloromethyl radical spin adduct might be reduced to an EPR-silent hydroxylamine form. Addition of 1.5 mM potassium ferricyanide as a mild oxidant led to a more than 20-fold increase in the signal shown in FIG. 6, while addition of 10 mM potassium ferricyanide caused most of the trapped signal to disappear.

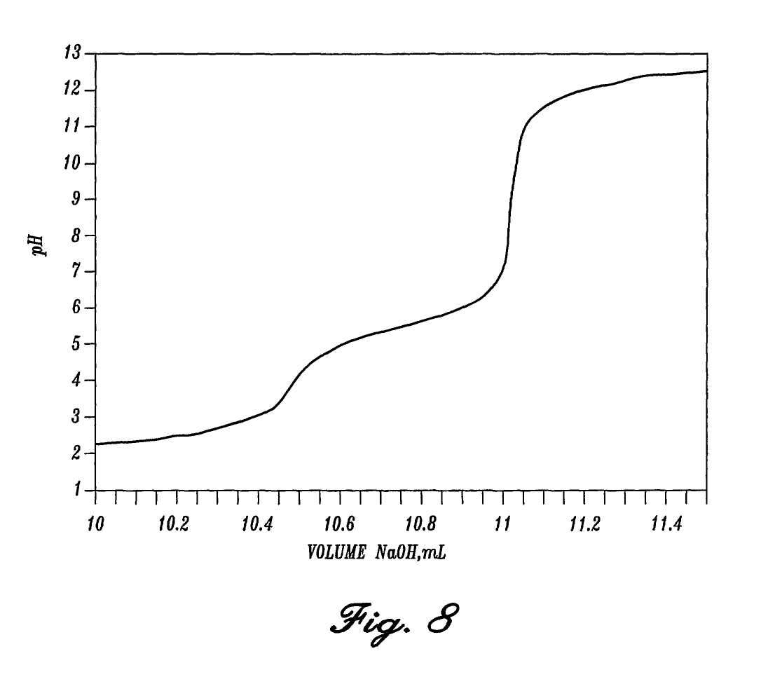

Another approach to the identification of possible radical intermediates included the use of the stable free radical 2,2,6,6-tetramethylpiperidine-N-oxyl (TEMPO) in reaction mixtures. Positive electrospray ionization MS showed the presence of 2,2,6,6-tetramethylpiperidinium cation (m/z=142) and a cation corresponding to a tetramethylpiperidine fragment (m/z=140) (see FIG. 8). Both of those species would be predicted from reactions in which a thiyl radical had condensed with the nitroxyl-containing compound (TEMPO) and decomposed through any of a variety of routes, either spontaneously or in the MS ion source.

In order to assess the viability of PDTC-dependent dechlorination as a remediation technology, it was useful to understand the underlying chemistry. The model presented can explain the product analysis and intermediate trapping data (Table 7) obtained from both P. stutzeri KC cultures and chemical reactions including PDTC and Cu[II]. This makes it possible to predict potential outcomes under various in situ conditions. One concern was that the process would not operate if iron concentrations were greater than approximately 10⁻⁵ M. The data indicate that iron should not directly inhibit dechlorination, that dechlorination instead would be limited by the amount of available copper. The amount of copper required to stimulate maximal dechlorination would be lower in the presence of metabolically active bacterial cells or reducing agents. The inhibition of dechlorination seen for ferric or ferrous iron with cultures of strain KC is therefore most likely the result of repression of PDTC synthesis, rather than a direct effect on the chemical transformation, a situation relevant to remediation since iron is likely to be more abundant than copper in most contaminated media. In addition, the use of engineered strains that are not subject to iron repression would be futile if the effect of iron was more direct. The production of so-called “exacerbating factors” by bacteria in contaminated media could confound efforts to use this transformation effectively; however, no evidence for this effect using strain KC cultures, culture supernatants, or another strain of the same species (Table 5) has been found.

TABLE 5
Effects of transition metal additions, bacterial
cells, and chemical reducing
agents on PDTC-dependent CCl₄ transformation

| | | No | | 0.5 mM | 0.5 mM |
|---|---|---|---|---|---|
| | | reductant^{a} | Cells^{b} | Sulfide^{a} | Ti[III]^{a} |
| No | PDTC | 10.3 ± 16.8 | 28.3 ± 1.8 | 209^{c} | 155.9 ± 4.8 |
| added | −PDTC | 0 | −0.4 ± 0.1 | 36.3 ± 7.4 | 1.3 ± 14.0 |
| metal | | | | | |
| Cu | PDTC | 109.8 ± 7.8 | 29 ± 0.2 | 209^{c} | 186.0 ± 0.3 |
| (II) | −PDTC | n.d. | n.d. | −16.2 ± 43.8 | 21.0 ± 8.6 |
| Fe | PDTC | 15.5 ± 14.7 | 29.6 ± 0.02 | 201 ± 9.6 | 118.5 ± 17.7 |
| (III) | −PDTC | n.d. | n.d. | −1.1 ± 11.2 | −0.9 ± 14.6 |
| Co | PDTC | −2.8 ± 1.9 | 0.7 ± 0.2 | 16.2 ± 9.4 | 92.0 ± 11.1 |
| (II) | −PDTC | n.d. | n.d. | 11.8 ± 2.1 | −4.7 ± 3.1 |
| Ni | PDTC | −1.0 ± 4.5 | 22.6 ± 4.6 | 204 ± 4.2 | 149.4 ± 4.7 |
| (II) | −PDTC | n.d. | n.d. | 3.7 ± 3.8 | 7.7 ± 3.3 |

Data are expressed as the mean ± standard deviation (n = 3) of the net removal of CCl₄ (nanomoles) and were obtained by subtracting the amount of CCl_{4 remaining in experimental vials from that remaining in blank vials containing only buffer and CCl}₄.
^{a}Reaction conditions: PDTC, 26 μM; indicated metals, 13 μM; indicated reductants, 0.5 mM; CCl_{4, approx. 200 nmoles; incubations were at 25° C. for 72 hours.}
^{b}Reaction conditions: PDTC, 50 μM; indicated metals, 25 μM; CCl_{4, approximately 50 nanomoles; incubations were at 25° C. for 16 hours.}
^{c}No CCl₄ remained in these experiments at the time of sampling
n.d.; not determined

TABLE 6
CCl₄ transformation by PDTC with or without
transition metals and chemical reducing agents^{a}

| | No reductant | | | 0.5 mM Na₂S | | | Ti(III) citrate | | |
|---|---|---|---|---|---|---|---|---|---|
| | CCl₄/mole^{b} | % CHCl₃ | % CS₂ | CCl₄/mole^{b} | % CHCl₃ | % CS₂ | CCl₄/mole^{b} | % CHCl₃ | % CS₂ |
| PDTCH₂ | 0.2 ± 0.3 | n.f. | n.f. | ≧4 | 4.5 ± 0.3 | 50.4 ± 2.9 | 3.1 ± 0.1 | 5.1 ± 0.8 | 6.3 ± 0.5 |
| Cu:PDTC | 2.2 ± 0.2 | <0.1 | 7.5 ± 0.8 | ≧4 | 0.9 ± 0.05 | 63.3 ± 5.2 | ≧3.7 | 0.2 ± 0.1 | 4.5 ± 0.5 |
| | (1.85 ± 0.1)* | | | (6.71 ± 0.1)* | | | | | |
| Fe:PDTC | 0.3 ± 0.3 | n.f. | n.f. | ≧4 | 5.2 ± 0.6 | 53.5 ± 4.6 | 2.4 ± 0.4 | 7.8 ± 0.9 | 8.7 ± 1.0 |
| Co:PDTC | <0.1 | n.f. | n.f. | 0.3 ± 0.2 | <0.7 | 3.9 ± 8.9 | 1.8 ± 0.2 | 34.2 ± 0.3 | n.f. |
| Ni:PDTC | <0.1 | n.f. | n.f. | ≧4 | 1.6 ± 0.3 | 39.6 ± 2.7 | 3.0 ± 0.1 | 2.4 ± 0.8 | 3.2 ± 0.9 |

^{a}The data are from reactions of Table 1.
^{b}nmoles of CCl₄ transformed per nanomole of PDTC
n.f., not found
*Values in parentheses were calculated from chloride determinations of reactions containing excess CCl₄.

TABLE 7
Recovery of ¹⁴C from ¹⁴CCl₄ after
reaction with Cu:PDTC

| | | | Non- | |
|---|---|---|---|---|
| | | Volatile, | strippable, | Total |
| | 14CO₂[₂?] | organic-trapped | aqueous | Recovery |
| Cu:PDTC, | 68.3 ± 1.0% | 20.0 ± 0.5% | 8.1 ± 0.2% | 96.4% |
| 50 μM | | | | |
| Cu:PDTC, | 16.4 ± 1.4% | 70.2 ± 4.2% | 1.3 ± 0.2% | 87.9% |
| 50 μM | | | | |
| 0.5 mM | | | | |
| Na2S | | | | |

Reactions were conducted in 1 ml 35 mM potassium phosphate buffer, pH 7.7, and included approximately 50 nmoles of ¹⁴CCl₄.

### Example 4

In this example, the stability constants and relative binding strengths for PDTC and several of the physiologically important metals that it binds are described.

Materials. All the commercial reagents were of the highest purity available and were used without further purification. Inductively Coupled Plasma Emission (ICP) standard metal stock solutions (1 g/L per metal ion) of Cu²⁺, Fe³⁺, Co³⁺, Ni²⁺, Zn²⁺, and Mn²⁺ were obtained from Fisher Scientific (FS). These standards contained 2% nitrate to keep the metals in solution. A Cr³⁺ stock solution was made with Cr₂(SO₄)₃ suspended in 2% HNO₃. PDTC was synthesized using the method of Hildebrand et al. PDTC stock solutions were prepared by weighing out 0.100 g of PDTC and diluting it volumetrically to 10 ml with dimethylformamide (DMF). Standardized NaOH (FS SS266-1) and HCl (FS SA48-1) solutions degassed in argon were used for titrations and pH adjustments. The spectrophotometric competition studies were carried out with stock solutions of disodium ethylenediaminetetraacetate dihydrate (EDTA) (Bio-Rad 161-0728), 2,6-pyridinedicarboxylic acid (DPA) (Aldrich P6, 380-8), and K₃FeCN₆ (Sigma P-83131).

General Instruments. Absorption spectra were recorded on a Hewlett-Packard 8453 UV/Visible diode array spectrophotometer control by a HP Pentium-class computer. A Fisher Scientific Accument Basic pH meter equipped with an Accument Basic pH/ATC combination silver/silver chloride reference electrode was used for pH measurements. Mass spectra were taken with an electrospray ionization tandem mass spectrometer (Quattro II, Micromass Ltd., UK). Volume-dependent titrations employed a 665 Dosimat Metrohm volume dispenser (Brinkman Instruments Inc. Westbury N.Y.).

Potentiometric Titrations. All solutions were prepared with deionized distilled water of better than 18 M Ohm resistance, and measurements were made at 25° C. The ionic strength in the titration experiments was fixed at 0.1 M with NaClO₄ (FS SS266-1). The electrode was calibrated to read pH according to the standard method. Potentiometric titration employed the use of the automatic dispenser and the pH meter. Argon-saturated solutions were titrated with 0.1 N NaOH. The titrated sample was continually purged with argon to minimize interference of air-derived CO₂. Temperature was maintained at 25° C.

Spectrophotometric Titrations. UV-Visible absorption spectra were recorded using a 1.0-cm path length quartz cell. Spectra were analyzed on a Hewlett Packard computer running UV-Visible Chemstation software (revision 52). Concentrations of metal ions and PDTC examined ranged from 10⁻⁴ to 10⁻⁵ M.

Ligand Protonation Constants. PDTC has three protonation sites, one on the pyridine nitrogen and two on carbonyl sulfur atoms. They are denoted LH₁, LH₂, and LH₃, respectively. Precipitation of PDTC will occur depending on PDTC concentration and pH. Precipitation is favored as the concentration of PDTC increases and pH approaches 2. The protonation constants for PDTC were determined by potentiometric titration (FIG. 8). As indicated by the titration, the pK₁ is 5.48 and pK₂ is 2.58, where:

$${K}_{n} = \frac{[ {LH}_{n} ]}{[ {H}^{+} ] [ {LH}_{n - 1} ]}$$

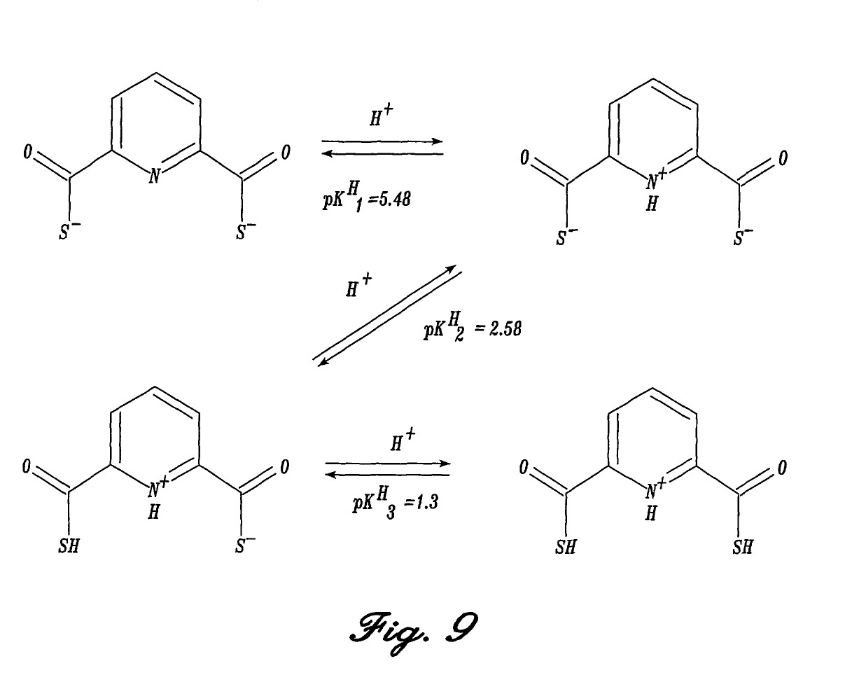

These constants were obtained using the computer program BigBest, which gave a sigma fit of 0.055. The third protonation constant is estimated to be 1.3. FIG. 9 shows the stepwise protonation of PDTC.

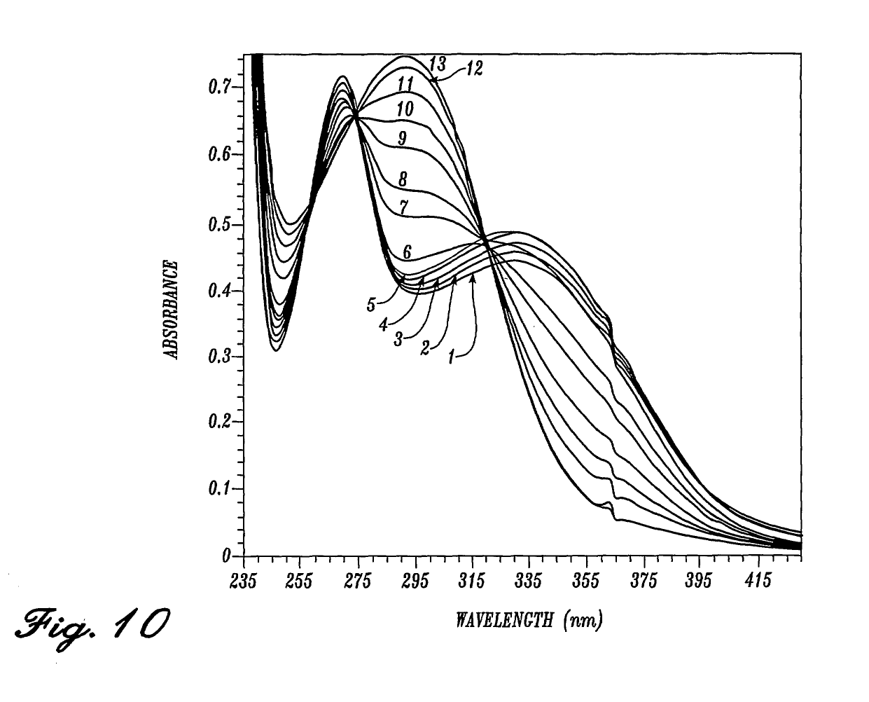

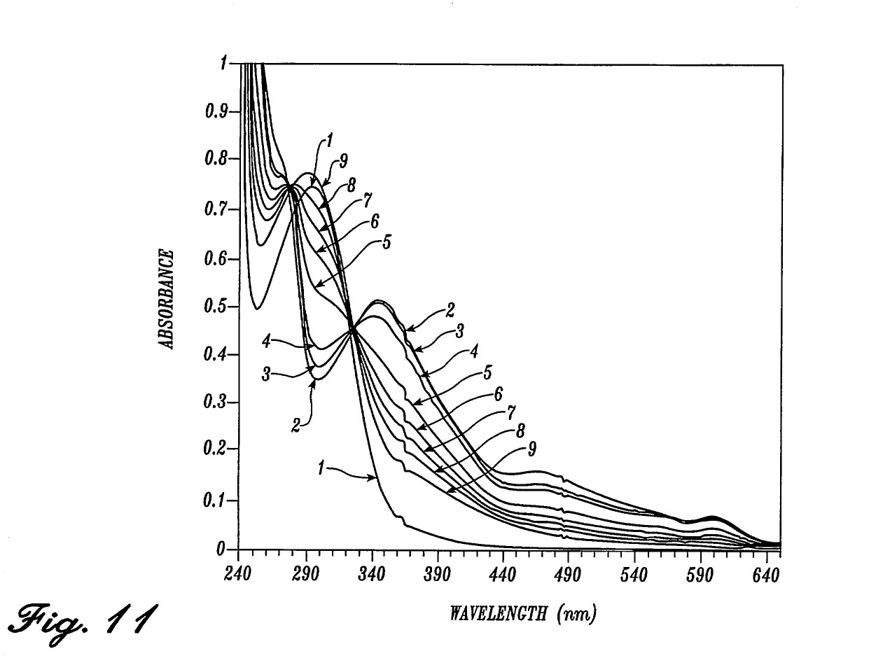

Changes in absorption spectra during titration of PDTC by NaOH are presented in FIG. 10. A profound color change near the first protonation constant was observed, and is probably due to the protonation of the nitrogen atom on the pyridine ring. PDTC absorption spectra from 230 to 430 nm were strongly affected as solution pH changed from acidic to basic.

Ferric Complexes. The first analysis of the stability constant for the PDTC ferric complex was done using spectrophotometric ligand-ligand competition methods. Solutions of EDTA, dipicolinic acid (DPA), and K₃FeCN₆ were prepared for competitive studies. Results showed that EDTA and DPA could not compete with PDTC. However, it was shown that PDTC could not compete with K₃FeCN₆ for iron. The results of these experiments indicated that the complex had an overall stability constant greater than the stability constant of 10¹⁶ for DPA, yet a weaker stability constant than the overall stability constant of 10⁵² for cyanide.

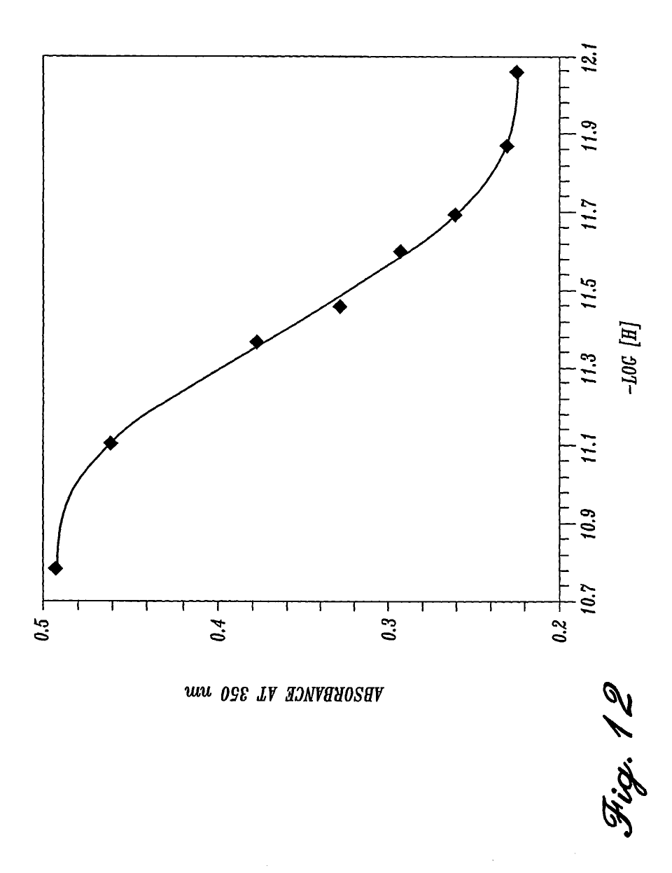

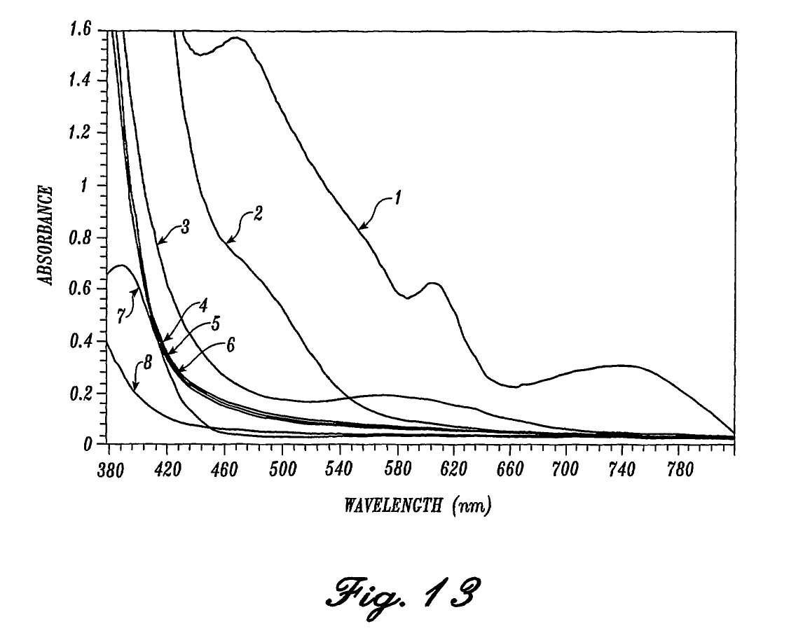

A titration curve of the ferric PDTC complex yielded a reversible spectral change over the pH range 11 to 12 (FIG. 10). With no other competing ligands present, hydroxide is the competing ligand causing a spectral change from the Fe(PDTC)₂ spectrum to the spectrum of free PDTC. Plotting the spectral change at a specific wavelength, fitting a curve, taking the derivative of this curve twice, and setting it equal to zero yields the point of inflection (FIG. 12), found to occur at pH=11.43.

At this pH, 2[PDTC]=[Fe(PDTC)₂]. Iron hydroxides are known to form the following complexes: Fe(OH)²⁺, Fe(OH₂)¹⁺, Fe(OH)₃, Fe(OH)₄¹⁻, Fe(OH)₅²⁻. To a negligible extent, they also form Fe₂(OH)₂⁴+ and Fe₃(OH)₄⁵⁺. The total amount of metal present is known, and can be defined by the following relation:

T_{Fe}_{³⁺}=[Fe(pdtc)₂¹⁻]+[Fe(OH)₁²⁺]+[Fe(OH)₂¹⁺]+[Fe(OH)₃]+[Fe(OH)₄¹⁻]+[Fe(OH)₅²⁻]+[Fe³⁺]

Overall formation constants for iron hydroxides have been previously determined:

$${β}_{xy} = \frac{{[ {{Fe}_{x} ( OH )}_{y}^{( 3 - y ) +} ] [ {H}^{+} ]}^{x}}{{[ {Fe}^{3 +} ]}^{x}} \qquad ( 1 )$$

And with the substitution of the above equations:

$$\begin{matrix}{T}_{{Fe}^{3 +}} = [ {Fe ( pdtc )}_{2}^{1 -} ] + \frac{{β}_{1} [ {Fe}^{3 +} ]}{[ {H}^{+} ]} + \frac{{β}_{12} [ {Fe}^{3 +} ]}{{[ {H}^{+} ]}^{2}} + \\ \frac{{β}_{13} [ {Fe}^{3 +} ]}{{[ {H}^{+} ]}^{3}} + \frac{{β}_{14} [ {Fe}^{3 +} ]}{{[ {H}^{+} ]}^{4}} + [ {Fe}^{3 +} ]\end{matrix} \qquad ( 2 )$$

Solving the above equation for the iron concentration:

$$[ {Fe}^{3 +} ] = \frac{( - {T}_{{Fe}^{3 +}} + [ {Fe ( pdtc )}_{2}^{1 -} ] ) \times {[ {H}^{+} ]}^{4}}{{{β}_{11} [ {H}^{+} ]}^{3} + {{β}_{12} [ {H}^{+} ]}^{2} + {β}_{13} [ {H}^{+} ] + {β}_{14} + {[ {H}^{+} ]}^{4}} \qquad ( 3 )$$

And knowing that iron forms the following complexes with PDTC:

$${K}_{11} = \frac{[ {( Fepdtc )}^{1 +} ]}{[ {Fe}^{3 +} ] [ {pdtc}^{2 -} ]} and {K}_{12} = \frac{[ {Fe ( pdtc )}_{2}^{1 -} ]}{[ {( Fepdtc )}^{1 +} ] [ {pdtc}^{2 -} ]} \qquad ( 3 , 4 )$$

Or in terms of overall stability constant:

$${β}_{12} = \frac{[ {Fe ( pdtc )}_{2}^{1 -} ]}{{[ {Fe}^{3 +} ] [ {pdtc}^{2 -} ]}^{2}} \qquad ( 5 )$$

With [PDTC], [Fe(PDTC)₂], and [Fe³⁺] known, β₁₂=β_{Fe+3} can now be calculated. The binding constant for the ferric PDTC complex, log β_{Fe+3}, was calculated to be 33.93.

Titrations with H₂SO₄. As shown in equation 5, the determination of β₁₂ depends on concentration of [pdtc²⁻]. The concentration of PDTC is also dependent upon the first, second, and third protonation constants:

$${K}_{n}^{H} = \frac{[ {H}_{n} pdtc ]}{[ {H}_{n - 1} pdtc ] [ {H}^{+} ]} \qquad ( 6 )$$

Therefore, the hydrogen ion competes with any metal PDTC complex. With successive additions of hydrogen ion, it becomes possible to find the point at which H⁺ outcompetes the metal ion. Since the protonation constants were previously determined, it becomes possible to estimate the stability constant based on the hydrogen concentration. The free iron concentration can be defined as:

$$[ {Fe}^{3 +} ] = {T}_{{Fe}^{3 +}} \times \frac{[ {Fe [ pdtc )}_{2}^{1 -} ]}{2} \qquad ( 7 )$$

A mass balance on the total ligand present yields:

T_{L}=2×[Fe(pdtc)₂¹⁻]+[pdtc²⁻]+[Hpdtc¹⁻]+[H₂pdtc]+[H₃pdtc¹⁺] (8)

Using equations 8 and 6 to solve for [PDTC²⁻]:

$$[ {pdtc}^{2 -} ] = \frac{- ( - {T}_{L} + 2 \times [ {Fepdtc}_{2}^{2 -} ] ) \times {K}_{1}^{H} \times {K}_{2}^{H} \times {K}_{3}^{H}}{( {K}_{1}^{H} \times {K}_{2}^{H} \times {K}_{3}^{H} + [ {H}^{+} ] \times {K}_{2}^{H} \times {K}_{3}^{H} + {[ {H}^{+} ]}^{2} + {[ {H}^{+} ]}^{3}} \qquad ( 9 )$$

The concentration of [Fe(PDTC)₂] was determined spectrophotometrically using a calibration curve at a wavelength of 605 nm. Now all three components of equation 5 are known from equations 7 and 9 and the [Fe(PDTC)₂] calibration curve. The log β₁₂ was found to be 32.49, but this value has several limitations for use. In order to approach a point where hydrogen ion could compete with iron, a concentration of 7-8 molar H₂SO₄ had to be reached. At an ionic strength this high, and because PDTC is believed to undergo acid hydrolysis at this pH, the number has little value in the determination of a stability constant. However, the number generated gives a fairly accurate estimate of the real stability constant at this extremely high ionic strength, and can be considered valuable for comparing the relative strengths of PDTC with other metal compounds. The same conditions were applied to cobalt, which gave a log β of 32.2.

Metal-Metal Competition. An array of metal solutions was set up for competition studies of metal versus metal. The experiment was set up so that PDTC could completely complex with either metal species present. Since Fe, Co, Ni, Mn and Cr form a 1:2 M(PDTC)₂ complex, the 0.25 mM solutions of two metals forming 0.5 mM were mixed with PDTC to reach a final concentration of 0.5 mM. The PDTC concentration in each bimetal solution was half that required to completely complex both metals. Table 8 shows how the experimental array was set up and also shows the dominant species for each metal in the metal competition experiment. The manganese and chromium spectra were too similar to conclude which species dominated. An equal sign indicates that both complexes for M(PDTC)₂ were present in similar proportion.

Metals with similar affinities for PDTC can be evaluated numerically for relative strengths. For metals with a 2:1 complex formation, the following relation can be used:

$${β}_{M2} = {β}_{M1} \frac{[ {M}_{1}^{{z}_{1}} ] [ {{M}_{2} ( pdtc )}_{2}^{{z}_{2} - 4} ]}{[ {M}_{2}^{{z}_{2}} ] [ {{M}_{1} ( pdtc )}_{2}^{{z}_{1} - 4} ]} \qquad ( 10 )$$

The above equation was used to find the stability constant for the [Co(PDTC)₂]¹⁻ complex. Since the [Fe(PDTC)₂]¹⁻ complex has a distinctive absorbance peak at 605 nm, the concentration this species was measured with a calibration curve (FIG. 12). A mass balance would then yield the following relation:

T_{M}_{^{z}}=[M(pdtc)₂^{z-4}]+[M^{z}] (11)

Using equation (10) to find each species concentration and then solving for β_{M2} in equation (11) yields the overall stability constant. It was found that log β_{Co}_{³⁺} was 33.45 and log β_{Ni}_{³⁺} was 33.56.

Potentiometric Titration. Results of the titration of PDTC show that pK₁ is 5.48 and that the pK₂ is 2.58. The program BigBest minimizes the standard deviations of fit over the entire titration curve by variation of the protonation constants. The error associated with the fit is relatively low, reported as SIGFIT=0.055. Error analysis of the third protonation constants was computed as 1.3. The determination of the third protonation constant for PDTC is an estimate at best, since titration methods are only valid at a pH range from 2 to 12 and the computation was out of the range of experimental data.

Spectrophotometric Titration. The challenge of measuring the overall equilibrium for PDTC was difficult, due to the nature of the proton dissociation constants and the high affinity of PDTC to the metals of interest. Competition studies for metabolic chelators often use EDTA as the competitor, even if EDTA has a lesser stability constant. This method relies on the fact that the complex has protonation sites with high affinities to the proton, and which also bind to the metal. As the pH is lowered, the apparent strength of the complex is weakened to a greater extent than is EDTA, so that at a certain pH, EDTA can outcompete the complex. If the pKa values of the ligand are low, and the stability constant is greater than that of EDTA, the ligand outcompetes EDTA at any pH. Thus, hydroxide was chosen as the competing ligand for measuring the stability constant.

Limitations on the accuracy of the determined stability constant included the fact that PDTC slowly undergoes base-catalyzed hydrolysis in a solution with basic pH. For this reason the time allowed for equilibrium to be reached was less then 10 minutes. It was noted, however, that very little change occurred over this time interval, indicating that the amount of time for equilibrium was sufficient.

The method used was verified by measuring the stability constant for EDTA under the same method and conditions. Hydroxide was found to compete with EDTA over the pH range of 6.5 to 9. Under the same titration conditions as in the iron(PDTC)₂ experiment, [EDTA] was found to equal [FeEDTA] at a pH of 7.63. The log K_{eff} for EDTA at this pH was calculated to be 14.9. The experimental log K_{eff} of EDTA was found to be 15.1. The calculated error of the log K_{eff FeEDTA} was then found to be 1.5%.

The results of the titration with copious amount of H₂SO₄ resulted in a log β_{Fe}_{³⁺} of 32.49 versus the log β_{Fe}_{³⁺} of 33.93. This 4% difference in log β_{Fe}_{³⁺} values is likely due to the different ionic strengths of the experiments and any possible error in the estimation of the third protonation constant.

Metal-Metal Competition Studies. The data from Table 8 can produce the following relative strengths of each metal (from strongest to weakest): Cu²⁺, Fe³⁺, Co³⁺>Ni²⁺>Zn²⁺>Mn²⁺, Cr³⁺.

Comparison with Other Iron Chelators. Reported stability constants of metabolically produced chelators can be very large. For example, enterobactin has an estimated log K_{ML} of 52. For the reasons mentioned previously, the chelating power of enterobactin is weakened as the pH is lowered. The low pKa values of PDTC, however, give it the ability to outcompete enterobactin below a pH of 6.6. A comparison of various chelators with PDTC is shown in Table 9.

TABLE 8
Results of cross competition metal/metal study for pdtc.
[Fe³⁺], [Co³⁺],
[Ni²⁺], [Cr³⁺] = 0.25 mM;
[Cu²⁺], [Zn²⁺],
[Mn²⁺] = 0.5 mM;
[pdtc] = 0.5 mM;
in 2% HNO₃ ; T = 25° C.
For ?, spectra too similar to determine
which metal complex was formed;
for =, both complexes were
present in similar proportions.

| | Co³⁺ | Ni²⁺ | Cu²⁺ | Zn²⁺ | Mn²⁺ | Cr³⁺ |
|---|---|---|---|---|---|---|
| Fe³⁺ | = | Fe³⁺ | = | Fe³⁺ | Fe³⁺ | Fe³⁺ |
| Co³⁺ | | = | = | Co³⁺ | Co³⁺ | Co³⁺ |
| Ni²⁺ | | | Cu²⁺ | Zn²⁺ | = | Ni²⁺ |
| Cu²⁺ | | | | Cu²⁺ | Cu²⁺ | Cu²⁺ |
| Zn²⁺ | | | | | Zn²⁺ | Zn²⁺ |
| Mn²⁺ | | | | | | ? |

TABLE 9
Log β values for various iron chelates

| Ligand | log β |
|---|---|
| Mecam | 35.6 |
| pdtc | 34.0 |
| Ferrioxamine E | 32.9 |
| Ferrochrome A | 32.0 |
| Ferrichrome | 29.1 |
| EDTA | 25.0 |
| DPTA | 27.6 |
| Ferrichrysin | 26.5 |
| EDTA | 25.0 |

In summary, the spectrophotometric and potentiometric titration studies on ferric PDTC show its strong affinity for Fe³⁺. Moreover, PDTC has been found to have comparable affinities for various other metals. The log stability constants for the iron III, cobalt III, and nickel II PDTC complexes have been found to be 33.93, 33.28, and 33.36, respectively. The first and second protonation constants for PDTC were found to be 5.48 and 2.58. The third pKa was estimated to be 1.3. These protonation constants and high affinity constants show that over a physiological pH range, ferric PDTC has one of the highest effective overall stability constants for metal binding among known bacterial chelators.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. An isolated nucleic acid molecule that is at least 70 percent identical to a nucleic acid molecule selected from the group consisting of SEQ ID NO:1, SEQ ID NO:3, SEQ ID NO:5, SEQ ID NO:7 and SEQ ID NO:13.

2. An isolated nucleic acid molecule of claim 1 wherein said isolated nucleic acid molecule is at least 90 percent identical to SEQ ID NO:1.

3. An isolated nucleic acid molecule of claim 1 wherein said isolated nucleic acid molecule is at least 90 percent identical to SEQ ID NO:3.

4. An isolated nucleic acid molecule of claim 1 wherein said isolated nucleic acid molecule is at least 90 percent identical to SEQ ID NO:5.

5. An isolated nucleic acid molecule of claim 1 wherein said isolated nucleic acid molecule is at least 90 percent identical to SEQ ID NO:7.

6. An isolated nucleic acid molecule of claim 1 wherein said isolated nucleic acid molecule is at least 90 percent identical to SEQ ID NO:13.

7. An isolated nucleic acid molecule of claim 1 wherein said isolated nucleic acid molecule consists of the nucleic acid sequence set forth in SEQ ID NO:1.

8. An isolated nucleic acid molecule of claim 1 wherein said isolated nucleic acid molecule consists of the nucleic acid sequence set forth in SEQ ID NO:3.

9. An isolated nucleic acid molecule of claim 1 wherein said isolated nucleic acid molecule consists of the nucleic acid sequence set forth in SEQ ID NO:5.

10. An isolated nucleic acid molecule of claim 1 wherein said isolated nucleic acid molecule consists of the nucleic acid sequence set forth in SEQ ID NO:7.

11. An isolated nucleic acid molecule of claim 1 wherein said isolated nucleic acid molecule consists of the nucleic acid sequence set forth in SEQ ID NO:13.

12. A vector comprising a nucleic acid molecule that is at least 70 percent identical to a nucleic acid molecule selected from the group consisting of SEQ ID NO:1, SEQ ID NO:3, SEQ ID NO:5, SEQ ID NO:7 and SEQ ID NO:13.

13. A vector of claim 12 wherein said vector comprises a nucleic acid molecule that is at least 70 percent identical to a nucleic acid molecule consisting of the sequence set forth in SEQ ID NO:1.

14. A vector of claim 12 wherein said vector comprises a nucleic acid molecule that is at least 70 percent identical to a nucleic acid molecule consisting of the sequence set forth in SEQ ID NO:3.

15. A vector of claim 12 wherein said vector comprises a nucleic acid molecule that is at least 70 percent identical to a nucleic acid molecule consisting of the sequence set forth in SEQ ID NO:5.

16. A vector of claim 12 wherein said vector comprises a nucleic acid molecule that is at least 70 percent identical to a nucleic acid molecule consisting of the sequence set forth in SEQ ID NO:7.

17. A vector of claim 12 wherein said vector comprises a nucleic acid molecule that is at least 70 percent identical to a nucleic acid molecule consisting of the sequence set forth in SEQ ID NO:13.

18. A host cell comprising a vector comprising a nucleic acid molecule that is at least 70 percent identical to a nucleic acid molecule consisting of a nucleic acid sequence selected from the group consisting of SEQ ID NO:1, SEQ ID NO:3, SEQ ID NO:5, SEQ ID NO:7 and SEQ ID NO:13.

19. A host cell of claim 18 comprising a vector comprising a nucleic acid molecule that is at least 70 percent identical to the nucleic acid molecule consisting of the nucleic acid sequence set forth in SEQ ID NO:1.

20. A host cell of claim 18 comprising a vector comprising a nucleic acid molecule that is at least 70 percent identical to the nucleic acid molecule consisting of the nucleic acid sequence set forth in SEQ ID NO:3.

21. A host cell of claim 18 comprising a vector comprising a nucleic acid molecule that is at least 70 percent identical to the nucleic acid molecule consisting of the nucleic acid sequence set forth in SEQ ID NO:5.

22. A host cell of claim 18 comprising a vector comprising a nucleic acid molecule that is at least 70 percent identical to the nucleic acid molecule consisting of the nucleic acid sequence set forth in SEQ ID NO:7.

23. A host cell of claim 18 comprising a vector comprising a nucleic acid molecule that is at least 70 percent identical to the nucleic acid molecule consisting of the nucleic acid sequence set forth in SEQ ID NO:13.

24. A host cell of claim 23 wherein said host cell is a member of the genus Pseudomonas.

25. A host cell of claim 23 wherein said host cell is a plant cell.

26. An isolated nucleic acid molecule comprising:
(a) a nucleic acid sequence that is at least 70 percent identical to the nucleic acid sequence set forth in SEQ ID NO: 1;
(b) a nucleic acid sequence that is at least 70 percent identical to the nucleic acid sequence set forth in SEQ ID NO: 3;
(c) a nucleic acid sequence that is at least 70 percent identical to the nucleic acid sequence set forth in SEQ ID NO: 5;
(d) a nucleic acid sequence that is at least 70 percent identical to the nucleic acid sequence set forth in SEQ ID NO: 14;
(e) a nucleic acid sequence that is at least 70 percent identical to the nucleic acid sequence set forth in SEQ ID NO: 16; and
(f) a nucleic acid sequence that is at least 70 percent identical to the nucleic acid sequence set forth in SEQ ID NO: 18.

27. An isolated nucleic acid molecule of claim 26 wherein said isolated nucleic acid molecule further comprises:
(a) a nucleic acid sequence that is at least 70 percent identical to the nucleic acid sequence set forth in SEQ ID NO:7;
(b) a nucleic acid sequence that is at least 70 percent identical to the nucleic acid sequence set forth in SEQ ID NO:9; and
(c) a nucleic acid sequence that is at least 70 percent identical to the nucleic acid sequence set forth in SEQ ID NO:11.

28. A vector comprising a nucleic acid molecule of claim 26.

29. A host cell comprising a vector of claim 28.

30. A host cell of claim 29 wherein said host cell is a member of the genus Pseudomonas.

31. A host cell of claim 29 wherein said host cell is a plant cell.

32. An isolated protein that is at least 70 percent identical to a protein consisting of an amino acid sequence selected from the group consisting of SEQ ID NO:2, SEQ ID NO:4, SEQ ID NO:6, SEQ ID NO:9.

33. A method for reducing the amount of a metal in a substrate, said method comprising the steps of:
(a) introducing into a substrate, said substrate comprising a metal ion species, a plant comprising roots and a PDTC gene cluster, the plant possessing a mechanism for transporting the metal ion species into the roots; and
(b) expressing the PDTC gene cluster in the plant roots to form PDTC under conditions that enable the plant to remove an amount of the metal ion species from the substrate that is greater than the amount of the metal ion species that the plant would remove in the absence of expression of the PDTC gene cluster in the plant roots.

34. A method for reducing the amount of a metal in the rhizosphere of a plant, said method comprising the steps of:
(a) introducing into the rhizosphere of a plant at least one bacterial species that comprises a PDTC gene cluster, the rhizosphere comprising a metal ion species and the plant possessing a mechanism that transports the metal ion from the rhizosphere into the plant roots; and
(b) culturing the at least one bacterial species in the rhizosphere for a time and under conditions that enable the bacterial species to synthesize PDTC and thereby increase availability of the metal ion to the plant roots so that the plant removes an amount of the metal ion from the substrate that is greater than the amount of the metal ion that the plant would remove if the bacterial species expressing PDTC was not present in the rhizosphere.

35. A method for degrading carbon tetrachloride in a substrate, said method comprising the steps of:
(a) introducing into a substrate a plant comprising roots and a PDTC gene cluster, the substrate further comprising Cu(II) ions and carbon tetrachloride that are introduced into the substrate, before, after or simultaneously with the introduction of the plant into the substrate; and
(b) expressing the PDTC gene cluster in the plant roots under conditions that enable the plant to synthesize PDTC and release the PDTC into the substrate, thereby chemically degrading the carbon tetrachloride by the action of a complex formed between the PDTC and the Cu(II) ions.

36. A method for degrading carbon tetrachloride in a substrate, said method comprising the steps of:
(a) introducing into a substrate, comprising Cu(II) ions and carbon tetrachloride, at least one bacterial species that comprises a PDTC gene cluster under conditions that enable expression of the PDTC gene cluster to form PDTC; and
(b) release of the PDTC into the substrate thereby chemically degrading the carbon tetrachloride by the action of a complex formed between the PDTC and the Cu(II) ions.

37. A method for degrading carbon tetrachloride in a substrate, said method comprising the steps of:
(a) introducing into a substrate, comprising carbon tetrachloride and Cu(II) ions, a nucleic acid molecule comprising a PDTC gene cluster comprising a plurality of PDTC biosynthetic genes, each of said PDTC biosynthetic genes being operably linked to at least one regulatory element that directs their expression within a microorganism, said nucleic acid molecule being attached to a particle;
(b) uptake of the introduced nucleic acid molecule by a microorganism;
(c) expression of the PDTC gene cluster within the microorganism to form PDTC; and
(d) release of the PDTC into the substrate thereby chemically degrading the carbon tetrachloride by the action of a complex formed between the PDTC and the Cu(II) ions.

38. A method for reducing the amount of a metal in a substrate, said method comprising the steps of:
(a) contacting PDTC with a substrate comprising a metal ion species; and
(b) allowing PDTC to form a metal complex with the metal ion species thereby reducing the amount of metal ion species in the substrate.

39. The method of claim 38, wherein the substrate is water.

40. The method of claim 38, wherein the substrate is soil.

41. The method of claim 38, wherein the metal ion comprises a heavy metal ion.

42. The method of claim 38, wherein the metal ion comprises a radionuclide.

43. The method of claim 38, wherein the metal ion is selected from the group consisting of transition metals, lanthanide metals, actinide metals, radionuclides, and heavy metals.

44. The method of claim 38 further comprising introducing a plant into the substrate, wherein the plant has an ability to take up the metal complex.

45. A method for degrading carbon tetrachloride in a substrate, said method comprising the steps of:
(a) contacting PDTC copper (II) complex with a substrate comprising carbon tetrachloride; and
(b) chemically degrading the carbon tetrachloride by the action of the complex.

46. The method of claim 45, wherein the substrate is water.

47. The method of claim 45, wherein the substrate is soil.

48. A method for immobilizing metal ions within a substrate, the method comprising the steps of:
(a) contacting PDTC with a substrate to form a metal complex with the metal ion species; and
(b) allowing PDTC to form a metal complex with the metal ion species thereby immobilizing the metal ion species
